(19) United States
(12) Patent Application Publication
(10) Pub. No.: US 2003/0229072 A1
(43) Pub. Date: Dec. 11, 2003

(54) Cyclic and acyclic amidines and pharmaceutical compositions containing them for use as progesterone receptor binning agents

(76) Inventors: William H Bullock, Easton, CT (US); Harold C E Kluender, Trumbull, CT (US); William L Collibee, Bethany, CT (US); Robert Dally, Indianapolis, IN (US); Martha E Rodriguez, Indianapolis, CO (US); Ming Wang, Milford, CT (US)

Correspondence Address: JEFFREY M. GREENMAN, BAYER PHARMACEUTICALS CORPORATION, 400 MORGAN LANE, WEST HAVEN, CT, 06516, US

(21) Appl. No.: 10/363,621

(22) PCT Filed: Aug. 30, 2001
(86) PCT No.: PCT/US01/27007

(30) Foreign Application Priority Data
Sep. 7, 2000 (US) 09656854

(51) Int. Cl. A61K 31/55; A61K 31/537; A61K 31/4747; A61K 31/44; C07D 209/54; C07D 215/38

(52) U.S. Cl. 514/214.01; 540/605; 540/598; 546/16; 546/159; 540/576; 548/408; 564/244; 540/609; 544/162; 546/229; 514/217.12; 514/217.11; 514/237.8; 514/317; 514/278; 514/409; 514/524; 514/637; 514/313

(57) ABSTRACT

Disclosed are cyclic and acyclic amidines, pharmaceutical compositions containing such amidines, and their use in treating or preventing progesterone receptor mediated diseases or conditions, such as osteopenia and osteoporosis.

### FIELD OF THE INVENTION

The invention relates to cyclic and acyclic compositions, more particularly cyclic and acyclic amidines, pharmaceutical compositions containing such amidines and their use in modulating progesterone receptor mediated processes.

### BACKGROUND

Steroidal and non-steroidal compounds which bind to the progesterone receptor may act as either agonists or antagonists and thereby have utility as pharmaceutical agents for treatment of a variety of medical conditions.

In particular, ligands to the progesterone receptor are known to play an important role in gynecological medicine, cancer, and prevention of osteoporosis. The natural ligand, the steroid progesterone and its synthetic analogs, are, for example, used in birth control formulations. Antagonists to progesterone are useful in treating chronic disorders such as certain forms of hormone-dependent cancer of the breast, ovaries, and endometrium, and in treating uterine fibroids. Endometriosis, a leading cause of infertility in women, is also amenable to treatment with progesterone. The steroidal progesterone analog, medroxyprogesterone, alone or in combination with estrogens, is indicated for prevention of osteoporosis, treatment of vulvar and/or vaginal atrophy, treatment of moderate to severe vasomotor symptoms associated with menopause, treatment of secondary amenorrehea, treatment of abnormal uterine bleeding due to hormonal imbalance in the absence of organic pathology, prevention of pregnancy, or as adjunctive therapy and palliative treatment of inoperable, recurrent, and metastatic endometrial or renal carcinoma (Merck Manual; Merck & Co. (1998)).

Mifepristone and onapristone, steroidal antagonists of progesterone, have been evaluated for use in the treatment of breast cancer, endometriosis and uterine fibroids, or as contraceptive agents. (Clin. Obstetr. Gynecol, 38(4), 921-934 (1995)).

Side-effects associated with steroidal compounds which bind to the progesterone receptor limit their therapeutic usefulness. Mifepristone has been reported to be not only a potent antiprogestin, but also an antiglucocorticoid and an anti-androgen.(Exp. Opin. Ther. Patents, 9(6), 695 (1999); Ann, NY Acad. Sci, 828,47-58 (1997)). Such compounds, being structurally similar to natural steroid ligands, may also interact with a number of other steroid receptors, either directly or following metabolism in vivo.

Non-steroidal ligands with specificity for the progesterone receptor can now be identified by in vitro assays and offer the potential advantage of having less cross-reactivity to other intracellular receptors. As a result, such non-steroidal ligands would be of significant value because of the reduced likelihood of undesirable side-effects in the medical therapies described above. Therefore, there remains a need for novel ligands of the progesterone receptor that are chemically accessible, possess high therapeutic specificity, and that do not cause the undesired side effects of steroidal ligands.

### SUMMARY OF THE INVENTION

The invention provides non-steroidal ligands with affinity for the progesterone receptor, particularly cyclic and acylic amidine compounds, which can act as progestins and/or antiprogestins, and thereby modulate progesterone receptor mediated processes. The invention further provides pharmaceutical compositions containing such compounds. Finally, the invention provides for methods of treating a mammal for diseases or conditions caused by progesterone receptor mediated processes.

The invention relates to compounds of the formula (I)

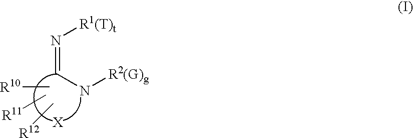

wherein

R¹

is selected from the group consisting of aryl of 6-12 carbon atoms and heteroaryl of 2-11 carbon atoms and 1-3 heteroatoms selected from the group consisting of N, S and O;

T

is selected from the group consisting of hydrogen, nitro, nitrile, alkyl of 1-6 carbon atoms, halogen, haloalkyl of 1-6 carbon atoms and a number of halogen atoms up to the perhalo level, aryl of 6-12 carbon atoms, and heteroaryl of 2-11 carbon atoms and 1-3 heteroatoms selected from the group consisting of N, S and O or

T

may form, together with a carbon atom adjacent to a carbon atom to which it is attached, a fused ring of 6-9 carbon atoms and 4-14 hydrogen atoms;

t

is 1-5;

R²

is selected from the group consisting of alkyl of 2-10 carbon atoms, haloalkyl of 1-10 carbon atoms and a number of halogen atoms up to the perhalo level, cycloalkyl of 3-12 carbon atoms, heterocycloalkyl of 4-7 carbon atoms and 1-3 heteroatoms selected from the group consisting of N, S and O alkenyl of 2-10 carbon atoms, cycloalkenyl of 5-12 carbon atoms and containing 1-3 rings, and alkynyl of 3-10 carbon atoms;

G

is a selected from the group consisting of hydrogen, nitro, nitrile, halogen, OH, OR⁴, ═O, haloalkyl of 1-4 carbon atoms and a number of halogen atoms up to the perhalo level, alkyl of 1-4 carbon atoms, alkenyl of 1-4 carbon atoms, cycloalkyl of 3-7 carbon atoms, heterocycloalkyl of 3-5 carbon atoms and 1-3 heteroatoms selected from the group consisting of N, O, and S, cycloalkenyl of 5-7 carbon atoms, heterocycloalkenyl of 4-6 carbon atoms and 1-3 heteroatoms selected from the group consisting of N, O, and S, CO₂R⁴, C(O)N(R⁵)(R⁶), aryl of 6-10 carbon atoms, heteroaryl of 3-9 carbon atoms and 1-3 heteroatoms selected from the group consisting of N, O, and S, S(O)_{y}R⁷, SO₃R⁷, and SO₂N(R⁵)(R⁶);

R⁴

is selected from the group consisting of alkyl of 1-4 carbon atoms, haloalkyl of 1-4 carbon atoms and a number of halogen atoms up to the perhalo level, cycloalkyl of 3-6 carbon atoms, and halocycloalkyl of 3-6 carbon atoms;

R⁵ and R⁶

are each independently selected from the group consisting of hydrogen and alkyl of 1-5 carbon atoms;

R⁷

is selected from the group consisting of alkyl of 1-5 carbon atoms, SO₂F, CHO, OH, nitro, nitrile, halogen, OCF₃, N-oxide, O—C(R⁸)₂O, C(O)NHC(O), the carbon atoms being connected to adjacent positions on R, and C(O)C₆H₄, the carbonyl carbon and the ring carbon ortho to the carbonyl carbon being connected to adjacent positions on R;

R⁸

is selected from the group consisting of hydrogen, halogen and alkyl of 1-4 carbon atoms;

y

is 0-2

g

is 0-4, with the exception of halogen, which may be employed up to the perhalo level;

provided that when G is alkyl of 1-4 carbon atoms, alkenyl of 1-4 carbon atoms, cycloalkyl of 3-7 carbon atoms, heterocycloalkyl of 3-5 carbon atoms, cycloalkenyl of 5-7 carbon atoms, or heterocycloalkenyl of 4-6 carbon atoms, then G optionally may bear secondary substituents of halogen up to the perhalo level; and when G is aryl or heteroaryl, then G optionally may bear secondary substituents independently selected from the group consisting of alkyl of 1-4 carbon atoms and halogen, the number of said secondary substituents being up to 3 for alkyl moieties, and up to the perhalo level for halogen;

X

forms, together with the nitrogen atom and carbon atom to which it is attached, a polycyclic ring structure of containing 3-4 rings, wherein each ring contains 3-8 carbon atoms and may optionally be substituted with one or more of alkyl of 1-6 carbon atoms or alkenyl of 2-6 carbon atoms, or

X is alkyl of 3-7 carbon atoms or alkenyl of 3-7 carbon atoms;

R¹⁰, R¹¹ and R¹²

(i) are each independently selected from the group consisting of hydrogen, haloalkyl of 1-10 carbon atoms and a number of halogen atoms up to the perhalo level, alkyl of 1-10 carbon atoms, cycloalkyl of 3-12 carbon atoms, alkenyl of 2-10 carbon atoms, cycloalkenyl of 5-7 carbon atoms, and alkynyl of 3-10 carbon atoms, or

(ii) R¹⁰ and R¹¹ are each independently selected from the group consisting of hydrogen, haloalkyl of 1-10 carbon atoms and a number of halogen atoms up to the perhalo level, alkyl of 1-10 carbon atoms, cycloalkyl of 3-12 carbon atoms, alkenyl of 2-10 carbon atoms, cycloalkenyl of 5-7 carbon atoms, and alkynyl of 3-10 carbon atoms and R¹² forms, together with the carbon atom to which it is attached, a spiro ring of 3-6 carbon atoms, or

(iii) R¹⁰ and R¹¹ are each independently selected from the group consisting of hydrogen, haloalkyl of 1-10 carbon atoms and a number of halogen atoms up to the perhalo level, alkyl of 1-10 carbon atoms, cycloalkyl of 3-12 carbon atoms, alkenyl of 2-10 carbon atoms, cycloalkenyl of 5-7 carbon atoms, and alkynyl of 3-10 carbon atoms and R¹² forms, together with the carbon atom adjacent to the carbon atom to which it is attached, a fused ring of 3-7 carbon atoms and 4-14 hydrogen atoms, or

(iv) R¹⁰ and R¹¹ are each independently selected from the group consisting of hydrogen, haloalkyl of 1-10 carbon atoms and a number of halogen atoms up to the perhalo level, alkyl of 1-10 carbon atoms, cycloalkyl of 3-12 carbon atoms, alkenyl of 2-10 carbon atoms, cycloalkenyl of 5-7 carbon atoms, and alkynyl of 3-10 carbon atoms and R¹² forms, together with the carbon atom that is 2-4 carbon atoms away from the carbon atom to which it is attached, a fused ring of 3-7 carbon atoms and 4-14 hydrogen atoms;

with the proviso that when X is alkyl of 3-4 carbon atoms and R¹⁰, R¹¹ and R¹² are all hydrogen:

t is 2-5;

at least one of T is 4-nitro or 4-nitrile and at least one other T is 2-alkyl, 2-halogen or 2-trifluoromethyl;

and R¹ is phenyl;

and with the further proviso that when X is alkyl of 3-7 carbon atoms or alkenyl of 3-7 carbon atoms and R¹⁰, R¹¹ and R¹² are each independently selected from the group consisting of hydrogen, haloalkyl of 1-10 carbon atoms and a number of halogen atoms up to the perhalo level, alkyl of 1-10 carbon- atoms; cycloalkyl of 3-12 carbon atoms, alkenyl of 2-10 carbon atoms, cycloalkenyl of 5-7 carbon atoms, and alkynyl of 3-10 carbon atoms, then at least one of T is nitro, nitrile, trifluoromethyl or halogen;

and pharmaceutically acceptable salts thereof.

The invention further relates to compounds of the formula (II)

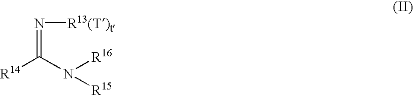

wherein

R¹³

is selected from the group consisting of aryl of 6-12 carbon atoms and 4-pyridyl;

R¹⁴

is selected from the group consisting of aryl of 6-12 carbon atoms, heteroaryl of 2-11 carbon atoms and 1-3 heteroatoms selected from the group consisting of N, S and O alkyl of 1-10 carbon atoms, cycloalkyl of 3-12 carbon atoms, heterocycloalkyl of 4-7 carbon atoms and 1-3 heteroatoms selected from the group consisting of N, S and O alkenyl of 2-10 carbon atoms, cycloalkenyl of 5-12 carbon atoms, with the proviso that said cycloalkenyl is mono-cyclic, and R¹⁷—R¹⁸;

T′

is selected from the group consisting of hydrogen, nitro, nitrile, alkyl of 1-6 carbon atoms, halogen, haloalkyl of 1-6 carbon atoms and a number of halogen atoms up to the perhalo level, aryl of 6-12 carbon atoms, and heteroaryl of 2-11 carbon atoms and 1-3 heteroatoms selected from the group consisting of N, S and O with the proviso that when R¹³ is aryl of 6-12 carbon atoms, at least one of T′ is nitro, nitrile, trifluoromethyl or halogen, or

T′

may form, together with a carbon atom adjacent to a carbon atom to which it is attached, a fused ring of 6-9 carbon atoms and 4-14 hydrogen atoms;

t′

is 1-5;

R¹⁷

is selected from the group consisting of alkyl of 1-10 carbon atoms and alkenyl of 2-10 carbon atoms;

R¹⁸

is selected from the group consisting of aryl of 6-12 carbon atoms, heteroaryl of 2-11 carbon atoms and 1-3 heteroatoms selected from the group consisting of N, S and O cycloalkyl of 3-12 carbon atoms, heterocycloalkyl of 4-7 carbon atoms and 1-3 heteroatoms selected from the group consisting of N, S and O, and cycloalkenyl of 5-12 carbon atoms;

R¹⁵ and R¹⁶

(i) are each independently selected from the group consisting of hydrogen, aryl of 6-12 carbon atoms, heteroaryl of 2-11 carbon atoms and 1-3 heteroatoms selected from the group consisting of N, S and O alkyl of 1-10 carbon atoms, cycloalkyl of 3-12 carbon atoms, heterocycloalkyl of 4-7 carbon atoms and 1-3 heteroatoms selected from the group consisting of N, S and O alkenyl of 2-10 carbon atoms, cycloalkenyl of 5-12 carbon atoms, and R¹⁹—R²⁰, such that the total of atoms in R¹⁴, R¹⁵ and R¹⁶ is greater than or equal to 9, or

(ii) are joined to form, together with the nitrogen atom to which they are attached, a 5-8 membered ring containing 4-7 carbon atoms and 1-2 heteroatoms selected from the group consisting of N, S and O which ring may optionally be substituted with R²¹ and R²², with the proviso that when R¹⁵ and R¹⁶ form a morpholine ring together with the nitrogen atom to which they are attached, said morpholine ring is substituted with at least one of R²¹ and R²²;

R¹⁹

is selected from the group consisting of alkyl of 1-10 carbon atoms, cycloalkyl of 3-12 carbon atoms, heterocycloalkyl of 4-7 carbon atoms and 1-3 heteroatoms selected from the group consisting of N, S and O alkenyl of 2-10 carbon atoms, cycloalkenyl of 5-12 carbon atoms, and alkynyl of 3-10 carbon atoms;

R²⁰

is selected from the group consisting of hydrogen, aryl of 6-12 carbon atoms, heteroaryl of 2-11 carbon atoms and 1-3 heteroatoms selected from the group consisting of N, S and O cycloalkyl of 3-12 carbon atoms, heterocycloalkyl of 4-7 carbon atoms and 1-3 heteroatoms selected from the group consisting of N, S and O cycloalkenyl of 5-12 carbon atoms, and R²³—R²⁴, with the proviso that when R²⁰ is phenyl, only one of R¹⁵ and R¹⁶ can be R¹⁹—R²⁰;

R²³

is selected from the group consisting of aryl of 6-12 carbon atoms and heteroaryl of 2-11 carbon atoms and 1-3 heteroatoms selected from the group consisting of N, S and O;

R²⁴

is selected from the group consisting of hydrogen, halogen, nitrile, nitro, alkyl of 1-10 carbon atoms, and haloalkyl of 1-6 carbon atoms and 1-3 halo atoms;

R²¹ and R²²

(i) are each independently selected from the group consisting of hydrogen, haloalkyl of 1-10 carbon atoms and a number of halogen atoms up to the perhalo level, alkyl of 1-10 carbon atoms, aryl of 6-12 carbon atoms, heteroaryl of 2-11 carbon atoms and 1-3 heteroatoms selected from the group consisting of N, S and O and benzimidazolinone, or

(ii) each independently forms, together with the carbon atom adjacent to the carbon atom to which it is attached, a fused ring of 3-6 carbon atoms and 4-10 hydrogen atoms, or

(iii) R²¹ is selected from the group consisting hydrogen, haloalkyl of 1-10 carbon atoms and a number of halogen atoms up to the perhalo level, alkyl of 1-10 carbon atoms, aryl of 6-12 carbon atoms, heteroaryl of 2-11 carbon atoms and 1-3 heteroatoms selected from the group consisting of N, S and O and benzimidazolinone and R²² forms, together with the carbon atom adjacent to the carbon atom to which it is attached, a fused ring of 3-6 carbon atoms and 4-10 hydrogen atoms;

and pharmaceutically acceptable salts thereof.

The invention further relates to pharmaceutical compositions containing any of the above-described compounds of formula (I) or (II) and a pharmaceutically acceptable carrier.

The invention also provides methods for treating a disease or condition in a mammal, wherein the effect to be achieved is:

A1) enhancement of bone formation in bone weakening diseases for the treatment or prevention of osteopenia or osteoporosis;

A2) enhancement of fracture healing;

B1) use as a female contragestive agent;

B2) prevention of endometrial implantation;

B3) induction of labor;

B4) treatment of luteal deficiency;

B5) enhanced recognition and maintanence of pregnancy

B6) counteracting of preeclampsia, eclampsia of pregnancy, and preterm labor;

B7) treatment of infertility, including promotion of spermatogenesis, induction of the acrosome reaction, maturation of oocytes, or in vitro fertilization of oocytes;

C1) treatment of dysmenorrhea;

C2) treatment of dysfunctional uterine bleeding;

C3) treatment of ovarian hyperandrogynism;

C4) treatment of ovarian hyperaldosteronism;

C5) alleviation of premenstral syndrome and of premenstral tension;

C6) alleviation of perimenstrual behavior disorders;

C7) treatment of climeracteric disturbance, including. menopause transition, mood changes, sleep disturbance, and vaginal dryness;

C8) enhancement of female sexual receptivity and male sexual receptivity;

C9) treatment of post menopausal urinary incontinence;

C10) improvement of sensory and motor functions;

C11) improvement of short term memory;

C12) alleviation of postpartum depression;

C13) treatment of genital atrophy;

C14) prevention of postsurgical adhesion formation;

C15) regulation of uterine immune function;

C16) prevention of myocardial infarction;

D1) hormone replacement;

E1) treatment of cancers, including hormone mediated cancers, such as breast cancer, uterine cancer, ovarian cancer, and endometrial cancer;

E2) treatment of endometriosis;

E3) treatment of uterine fibroids;

F1) treatment of hirsutism;

F2) inhibition of hair growth;

G1) activity as a male contraceptive;

G2) activity as an abortifacient; and

H1) promotion of mylin repair.

A method of the invention therefore provides for administering to a mammal an effective amount of a compound of the formula (III)

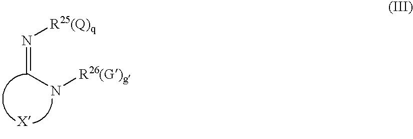

wherein

R²⁵

is selected from the group consisting of aryl of 6-12 carbon atoms and heteroaryl of 2-11 carbon atoms and 1-3 heteroatoms selected from the group consisting of N, S and O;

Q

is selected from the group consisting of hydrogen, nitro, nitrile, alkyl of 1-6 carbon atoms, halogen, haloalkyl of 1-6 carbon atoms and a number of halogen atoms up to the perhalo level, aryl of 6-12 carbon atoms, and heteroaryl of 2-11 carbon atoms and 1-3 heteroatoms selected from the group consisting of N, S and O or

Q

may form, together with a carbon atom adjacent to a carbon atom to which it is attached, a fused ring of 6-9 carbon atoms and 4-14 hydrogen atoms;

q

is 0-5;

R²⁶

is selected from the group consisting of hydrogen, alkyl of 1-10 carbon atoms, haloalkyl of 1-10 carbon atoms and a number of halogen atoms up to the perhalo level, cycloalkyl of 3-12 carbon atoms, heterocycloalkyl of 4-7 carbon atoms and 1-3 heteroatoms selected from the group consisting of N, S and O, alkenyl of 2-10 carbon atoms, cycloalkenyl of 5-12 carbon atoms and containing 1-3 rings, and alkynyl of 3-10 carbon atoms;

G′

is a selected from the group consisting of hydrogen, nitro, nitrile, halogen, OH, OR²⁷, ═O, haloalkyl of 1-4 carbon atoms and a number of halogen atoms up to the perhalo level, alkyl of 1-4 carbon atoms, alkenyl of 1-4 carbon atoms, cycloalkyl of 3-7 carbon atoms, heterocycloalkyl of 3-5 carbon atoms and 1-3 heteroatoms selected from the group consisting of N, O, and S, cycloalkenyl of 5-7 carbon atoms, heterocycloalkenyl of 4-6 carbon atoms and 1-3 heteroatoms selected from the group consisting of N, O, and S, CO₂R²⁷, C(O)N(R²⁸)(R²⁹), aryl of 6-10 carbon atoms, heteroaryl of 3-9 carbon atoms and 1-3 heteroatoms selected from the group consisting of N, O, and S, S(O)_{y}R³⁰, SO₃R³⁰, and SO₂N(R²⁸)(R²⁹);

R²⁷

is selected from the group consisting of alkyl of 1-4 carbon atoms, haloalkyl of 1-4 carbon atoms and a number of halogen atoms up to the perhalo level, cycloalkyl of 3-6 carbon atoms, and halocycloalkyl of 3-6 carbon atoms;

R²⁸ and R²⁹

are each independently selected from the group consisting of hydrogen and alkyl of 1-5 carbon atoms;

R³⁰

is selected from the group consisting of alkyl of 1-5 carbon atoms, SO₂F, CHO, OH, nitro, nitrile, halogen, OCF₃, N-oxide, O—C(R³¹)₂O, C(O)NHC(O), the carbon atoms being connected to adjacent positions on R, and C(O)C₆H₄, the carbonyl carbon and the ring carbon ortho to the carbonyl carbon being connected to adjacent positions on R;

R³¹

is selected from the group consisting of hydrogen, halogen and alkyl of 1-4 carbon atoms;

y

is 0-2

g′

is 0-4, with the exception of halogen, which may be employed up to the perhalo level;

provided that when G is alkyl of 1-4 carbon atoms, alkenyl of 1-4 carbon atoms, cycloalkyl of 3-7 carbon atoms, heterocycloalkyl of 3-5 carbon atoms, cycloalkenyl of 5-7 carbon atoms, or heterocycloalkenyl of 4-6 carbon atoms, then G optionally may bear secondary substituents of halogen up to the perhalo level; and when G is aryl or heteroaryl, then G optionally may bear secondary substituents independently selected from the group consisting of alkyl of 1-4 carbon atoms and halogen, the number of said secondary substituents being up to 3 for alkyl moieties, and up to the perhalo level for halogen;

X′

forms, together with the nitrogen atom and carbon atom to which it is attached, a polycyclic ring structure of containing 3-4 rings, wherein each ring contains 3-8 carbon atoms and may optionally be substituted with one or more of alkyl of 1-6 carbon atoms or alkenyl of 2-6 carbon atoms, or

X′ is

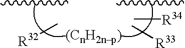

wherein binding is achieved via the terminal carbon atoms;

n

is 3-7;

p

is 0-7;

R³², R³³ and R³⁴

(i) are each independently selected from the group consisting of hydrogen, haloalkyl of 1-10 carbon atoms and a number of halogen atoms up to the perhalo level, alkyl of 1-10 carbon atoms, cycloalkyl of 3-12 carbon atoms, alkenyl of 2-10 carbon atoms, cycloalkenyl of 5-7 carbon atoms, and alkynyl of 3-10 carbon atoms, or

(ii) R³² and R³³ are each independently selected from the group consisting of hydrogen, haloalkyl of 1-10 carbon atoms and a number of halogen atoms up to the perhalo level, alkyl of 1-10 carbon atoms, cycloalkyl of 3-12 carbon atoms, alkenyl of 2-10 carbon atoms, cycloalkenyl of 5-7 carbon atoms, and alkynyl of 3-10 carbon atoms and R³⁴ forms, together with the carbon atom to which it is attached, a spiro ring of 3-6 carbon atoms, or

(iii) R³² and R³³ are each independently selected from the group consisting of hydrogen, haloalkyl of 1-10 carbon atoms and a number of halogen atoms up to the perhalo level, alkyl of 1-10 carbon atoms, cycloalkyl of 3-12 carbon atoms, alkenyl of 2-10 carbon atoms, cycloalkenyl of 5-7 carbon atoms, and alkynyl of 3-10 carbon atoms and R³⁴ forms, together with the carbon atom adjacent to the carbon atom to which it is attached, a fused ring of 3-7 carbon atoms and 4-14 hydrogen atoms, or

(iv) R³² and R³³ are each independently selected from the group consisting of hydrogen, haloalkyl of 1-10 carbon atoms and a number of halogen atoms up to the perhalo level, alkyl of 1-10 carbon atoms, cycloalkyl of 3-12 carbon atoms, alkenyl of 2-10 carbon atoms, cycloalkenyl of 5-7 carbon atoms, and alkynyl of 3-10 carbon atoms and R³⁴ forms, together with the carbon atom that is 2-4 carbon atoms away from the carbon atom to which it is attached, a fused ring of 3-7 carbon atoms and 4-14 hydrogen atoms;

and pharmaceutically acceptable salts thereof.

A method of the invention further provides for the treatment or prevention of a progesterone receptor mediated disease or condition by administering to a mammal an effective amount of a compound of the formula (IV)

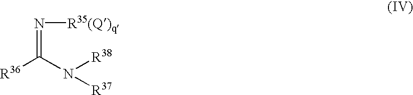

wherein

R³⁵

is selected from the group consisting of aryl of 6-12 carbon atoms and heteroaryl of 2-11 carbon atoms and 1-3 heteroatoms selected from the group consisting of N, S and O;

R³⁶

is selected from the group consisting of aryl of 6-12 carbon atoms, heteroaryl of 2-11 carbon atoms and 1-3 heteroatoms selected from the group consisting of N, S and O alkyl of 1-10 carbon atoms, cycloalkyl of 3-12 carbon atoms, heterocycloalkyl of 4-7 carbon atoms and 1-3 heteroatoms selected from the group consisting of N, S and O alkenyl of 2-10 carbon atoms, cycloalkenyl of 5-12 carbon atoms, and R³⁹—R⁴⁰;

Q′

is selected from the group consisting of hydrogen, nitro, nitrile, alkyl of 1-6 carbon atoms, halogen, haloalkyl of 1-6 carbon atoms and a number of halogen atoms up to the perhalo level, aryl of 6-12 carbon atoms, and heteroaryl of 2-11 carbon atoms and 1-3 heteroatoms selected from the group consisting of N, S and O or

Q′

may form, together with a carbon atom adjacent to a carbon atom to which it is attached, a fused ring of 6-9 carbon atoms and 4-14 hydrogen atoms;

q′

is 0-5;

R³⁹

is selected from the group consisting of alkyl of 1-10 carbon atoms and alkenyl of 2-10 carbon atoms;

R⁴⁰

is selected from the group consisting of aryl of 6-12 carbon atoms, heteroaryl of 2-11 carbon atoms and 1-3 heteroatoms selected from the group consisting of N, S and O cycloalkyl of 3-12 carbon atoms, heterocycloalkyl of 4-7 carbon atoms and 1-3 heteroatoms selected from the group consisting of N, S and O. and cycloalkenyl of 5-12 carbon atoms;

R³⁷ and R³⁸

(i) are each independently selected from the group consisting of hydrogen, aryl of 6-12 carbon atoms, heteroaryl of 2-11 carbon atoms and 1-3 heteroatoms selected from the group consisting of N, S and O alkyl of 1-10 carbon atoms, cycloalkyl of 3-12 carbon atoms, heterocycloalkyl of 4-7 carbon atoms and 1-3 heteroatoms selected from the group consisting of N, S and O alkenyl of 2-10 carbon atoms, cycloalkenyl of 5-12 carbon atoms, and R⁴¹—R⁴², such that the total of atoms in R³⁶, R³⁷ and R³⁸ is greater than or equal to 9, or

(ii) are joined to form, together with the nitrogen atom to which they are attached, a 5-8 membered ring containing 4-7 carbon atoms and 1-2 heteroatoms selected from the group consisting of N, S and O which ring may optionally be substituted with R⁴¹ and R⁴²;

R^{41 is selected from the group consisting of alkyl of} 1-10 carbon atoms, cycloalkyl of 3-12 carbon atoms, heterocycloalkyl of 4-7 carbon atoms and 1-3 heteroatoms selected from the group consisting of N, S and O alkenyl of 2-10 carbon atoms, cycloalkenyl of 5-12 carbon atoms, and alkynyl of 3-10 carbon atoms;

R⁴²

is selected from the group consisting of hydrogen, aryl of 6-12 carbon atoms, heteroaryl of 2-11 carbon atoms and 1-3 heteroatoms selected from the group consisting of N, S and O cycloalkyl of 3-12 carbon atoms, heterocycloalkyl of 4-7 carbon atoms and 1-3 heteroatoms selected from the group consisting of N, S and O cycloalkenyl of 5-12 carbon atoms, and R⁴⁵—R⁴⁶;

R⁴³

is selected from the group consisting of aryl of 6-12 carbon atoms and heteroaryl of 2-11 carbon atoms and 1-3 heteroatoms selected from the group consisting of N, S and O;

R⁴⁶

is selected from the group consisting of hydrogen, halogen, nitrile, nitro, alkyl of 1-10 carbon atoms, and haloalkyl of 1-6 carbon atoms and 1-3 halo atoms;

R⁴³ and R⁴⁴

(i) are each independently selected from the group consisting of hydrogen, haloalkyl of 1-10 carbon atoms and a number of halogen atoms up to the perhalo level, alkyl of 1-10 carbon atoms, aryl of 6-12 carbon atoms, heteroaryl of 2-11 carbon atoms and 1-3 heteroatoms selected from the group consisting of N, S and O and benzimidazolinone, or

(ii) each independently forms, together with the carbon atom adjacent to the carbon atom to which it is attached, a fused ring of 3-6 carbon atoms and 4-10 hydrogen atoms, or

(iii) R⁴³ is selected from the group consisting hydrogen, haloalkyl of 1-10 carbon atoms and a number of halogen atoms up to the perhalo level, alkyl of 1-10 carbon atoms, aryl of 6-12 carbon atoms, heteroaryl of 2-11 carbon atoms and 1-3 heteroatoms selected from the group consisting of N, S and O and benzimidazolinone and R⁴⁴ forms, together with the carbon atom adjacent to the carbon atom to which it is attached, a fused ring of 3-6 carbon atoms and 4-10 hydrogen atoms;

and pharmaceutically acceptable salts thereof.

The present invention therefore provides non-steriodal compounds, pharmaceutical compositions containing such compounds and methods for the treatment or prevention of progesterone receptor mediated diseases and conditions. Compounds, compositions and methods of the present invention therefore are useful in treatment of progesterone receptor mediated diseases and conditions without the concommitant undesired side-effects associated with known treatments that use steroid compounds. These and other aspects of the invention will be more apparent from the following description and claims.

### DETAILED DESCRIPTION OF THE INVENTION

The invention provides novel, non-steroidal compounds, namely cyclic and acyclic amidines, pharmaceutical compositions containing such compounds, and their use for the treatment or prevention of progesterone receptor mediated diseases or conditions. The invention further provides methods of treating or preventing progesterone receptor mediated diseases or conditions in mammals, such as humans, by administration of a non-steroidal compound according to any one of formulas I-IV, each of which has been broadly described above in the summary.

In preferred embodiments of the compounds of the general formula I:

R¹ is aryl of 6-12 carbon atoms and is more preferably phenyl,

R² is alkyl of 2-10 carbon atoms, alkenyl of 2-10 carbon atoms or cycloalkyl of 3-12 carbon atoms,

G is hydrogen,

g is the number of subsituents G and is 0-4,

T is a substituent on R¹ and is selected from nitro, nitrile, trifluormethyl and halogen,

t is the number of substituents T and is 1 or 2,

X is alkyl of 3-7 carbon atoms or alkenyl of 3-7 carbon atoms, or X is a polycyclic ring structure of 3-4 rings, and

R¹⁰, R¹¹ and R¹² are independently selected from hydrogen and alkyl of 1-10 carbon atoms.

As used herein, the term “aryl” includes aromatic ring structures that are substituents on another atom. These aryls may also be substituted with substituents, such as nitrile, nitro, halogen, haloalkyl, etc. Non-limiting examples of aryls include phenyl, napthyl, etc. Likewise, the term “heteroaryl” as used herein includes aromatic ring structures containing between one and three heteroatoms, such as O, N and S, that are substituents on another atom. These heteroaryls may also be substituted with substituents, such as nitrile, nitro, halogen, haloalkyl, etc. Non-limiting examples of heteroaryls include pyridyl, furyl, quinolyl, etc.

In preferred compounds of formula I, X may either be an alkyl or alkenyl chain or it may combine with the nitrogen and carbon atoms to which it is attached to form a polycyclic ring structure having 3 or 4 rings. As used herein the term “alkyl” includes straight-chain or branched alkyls of between 1 and 10 carbon atoms. The term “alkenyl” includes straight-chain or branched alkenyls of between 2 and 10 carbon atoms. As used herein the term “alkynyl” includes straight-chain or branched alkynyls of between 2 and 10 carbon atoms. Preferred compounds of formula I in which X is alkyl or alkenyl include:

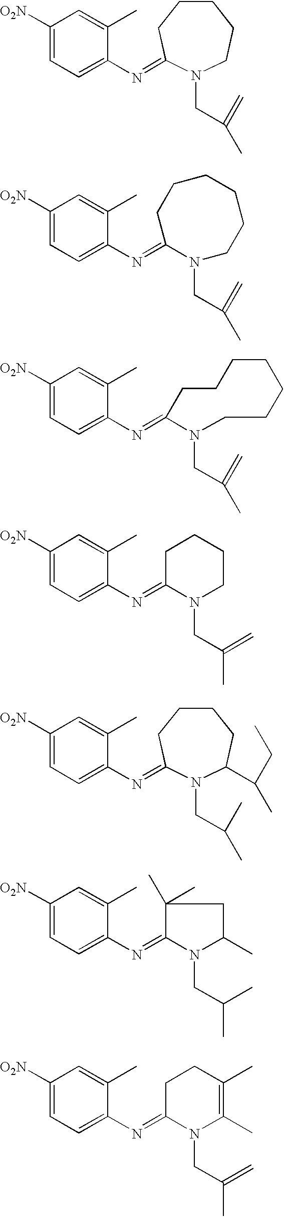

More preferred compounds of the invention include those in which R¹ is phenyl. Also preferred are those compounds for which the substituent T is at the para position on the phenyl ring.

The polycyclic ring structures containing 3 or 4 rings in compositions of the invention have rings which each contain between 3 and 8 carbon atoms and a total of 8-20 carbons. These rings may each be optionally substituted with 1-3 alkyl groups of 1-6 carbon atoms and/or 1-3 alkenyl groups of 2-6 carbon atoms. Examples of polycyclic ring structures in compositions of the invention include but are not limited to the following:

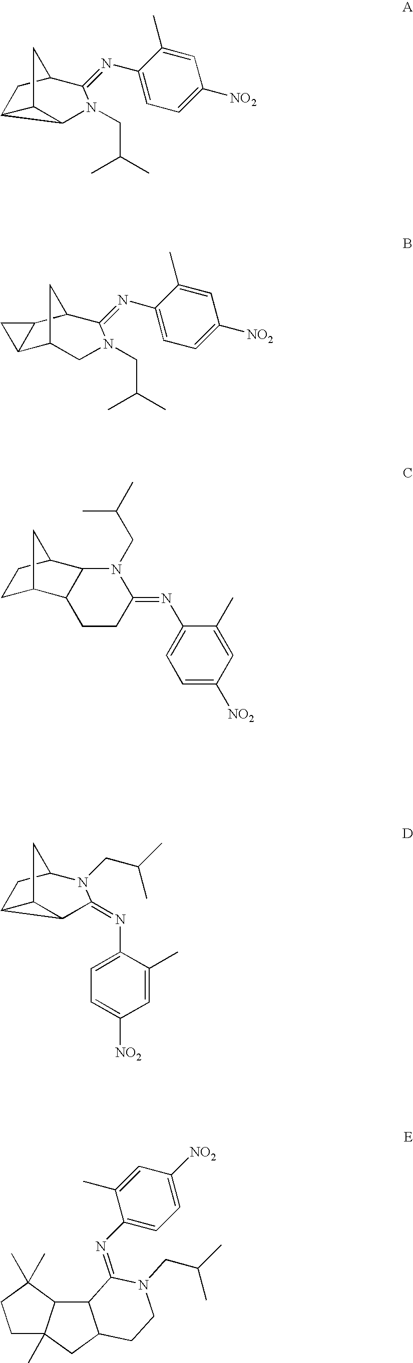

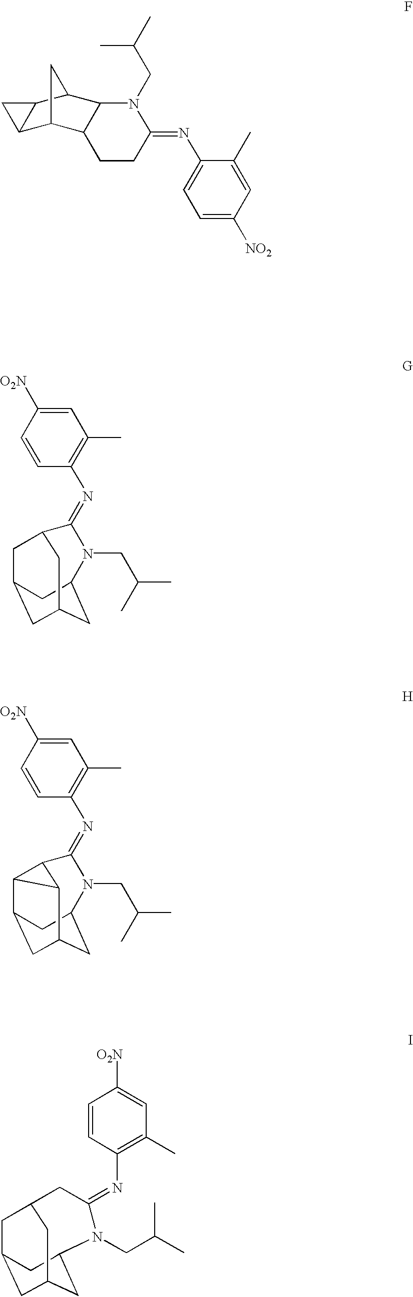

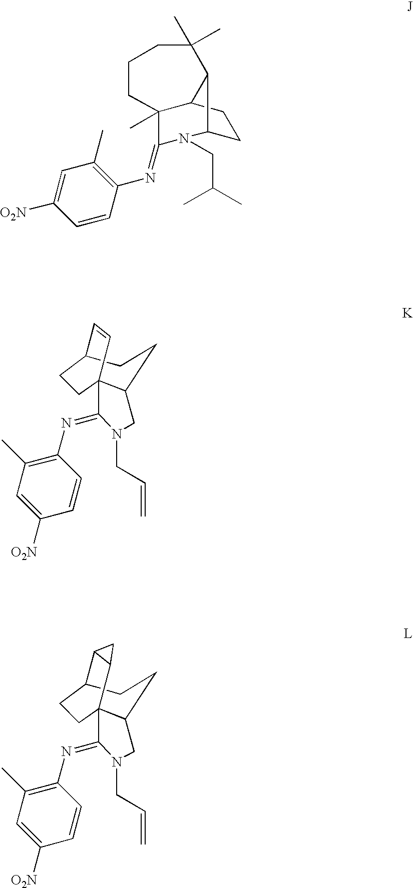

All of the above-listed compounds A-L can be prepared from the ketone, as illustrated in Flow Diagram IX below. For example, compound A (3-aza-4-[aza(2-methyl-4-nitrophenyl)methylene]-3-(2-methylpropyl)tricyclo[3.2.1.0<2,7>]octane) can be prepared from the amide 3-azatricyclo[3.2.1.0<2,7>]octan-4-one; Magn. Reson. Chem. 1987, 25, 443. Compound B(7-aza-6-[aza(2-methyl-4-nitrophenyl)methylene]-7-(2-methylpropyl)tricyclo[3.3.1.02,4>]nonane) can be prepared from the ketone tricyclo[3.2.1.02,4]octan-6-one; J Organomet. Chem. 1985, 281, 397. Compound C (3-aza-4-[aza(2-methyl-4-nitrophenyl)methylene]-3-(2-methylpropyl)tricyclo[6.2.1.0<2,7>]undecane) can be prepared from the amide 3-azatricyclo[6.2.1.0<2,7>]undecan-4-one; German Patent DE 3,242,151 (1984). Compound D (4-aza-3-faza(2-methyl-4-nitrophenyl)methylene]-4-(2-methylpropyl)tricyclo[3.3.1.0<2,8>]nonane) can be prepared from the amide 4-azatricyclo[3.3.1.0<2,8>]nonan-3-one; Magn. Reson. Chem. 1987, 25, 443. Compound E (11-aza-12-[aza(2-methyl4-nitrophenyl)methylene]-3,3,6-trimethyl-11-(2-methylpropyl)tricyclo[6.4.0.0<2,6>]dodecane) can be prepared from the ketone decahydro-4,4,6a-trimethyl-3H-cyclopenta[a]pentalen-3-one; J Am. Chem. Soc. 1984, 106, 7500. Compound G (4-aza-5-[aza(2-methyl-4-nitrophenyl)methylene]-4-(2-methylpropyl)tricyclo[4.3.1.1<3,8>]undecane) can be prepared from the commercially available amide 5-azatricyclo[4.3.1.1<3,8>]undecan-4-one. Compound H (6-aza-5-[aza(2-methyl-4-nitrophenyl)methylene]-6-(2-methylpropyl)tetracyclo[5.3.1.0<2,4>0.0<3,9>]undecane) can be prepared from the ketone octahydro-2,4-methano-3H-cycloprop[cd]inden-3-one; J Org. Chem. 1975, 40, 1079. Compound I (4-aza-5-[aza(2-methyl-4-nitrophenyl)methylene]-4-(2-methylpropyl)tricyclo[5.3.1.1<3,9>]dodecane) can be prepared from the amide 4-azatricyclo[5.3.1.1<3,9>]dodecan-5-one; J Org. Chem. 1972, 37, 3961. Compound J (9-aza-8-[aza(2-methyl-4-nitrophenyl)methylene]-3,3,7-trimethyl-9-(2-methylpropyl)tricyclo[5.5.0.0<2,10>]dodecane) can be prepared from the amide 9-aza-3,3,7-trimethyltricyclo[5.5.0.0<2,10>]dodecan-8-one; Indian J. Chem. 1972, 10, 315. Compound K (3-aza-2-[aza(2-methyl-4-nitrophenyl)methylene]-3-prop-2-enyltricyclo[6.2.2.0<1,5>]dodec-9-ene) can be prepared from the amide 3-aza-3-prop-2-enyltricyclo[6.2.2.0<1,5>]dodec-9-en-2-one; Tetrahedron Lett. 1976, 4517.

Examples of preferred compounds of formula I in which X is a polycyclic ring structure of 3-4 rings include:

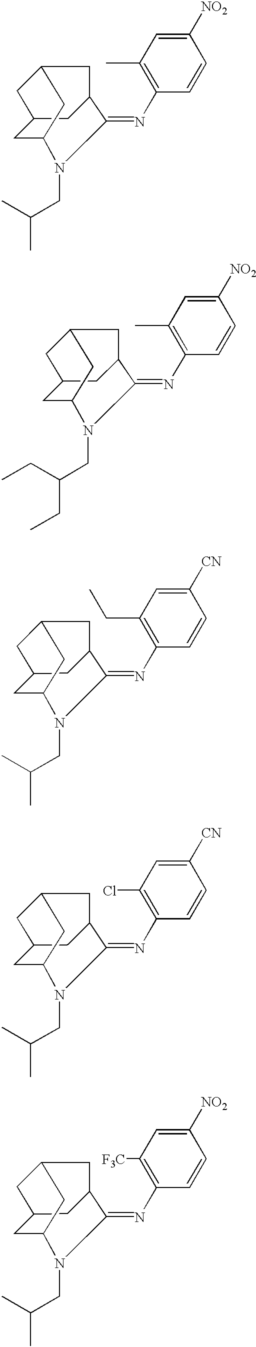

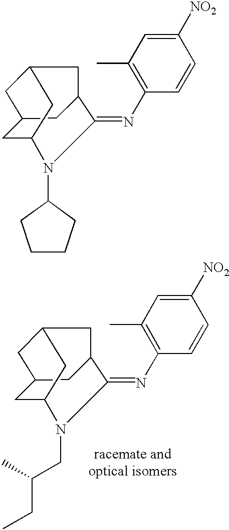

In other embodiments of the compounds of formula I:

1) any one of R¹⁰, R¹¹ and R¹² forms a Spiro ring together with the carbon atom to which it is attached. The spiro ring contains between 3 and 6 carbon atoms.

Examples of preferred compounds where one of R¹⁰, R¹¹ and R¹² forms a spiro ring include:

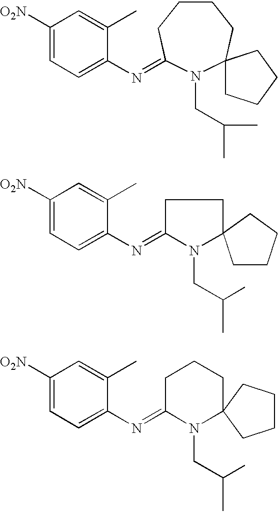

2) any one of R¹⁰, R¹¹ and R¹² forms a fused ring together with the ring containing X. The ring may form together with the carbon atom adjacent to the one to which R¹⁰, R¹¹ or R¹² is attached or it may form together with the carbon atoms that is 2-4 carbon atoms away from the carbon atom to which R¹⁰, R¹¹ or R¹² is attached.

Examples of preferred compounds where R¹⁰, R¹¹ and R¹² form fused rings with the ring containing X include:

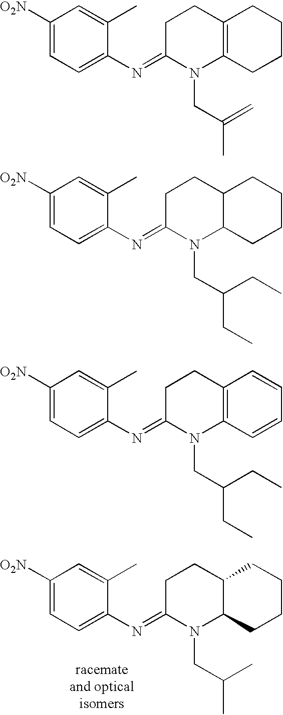

T forms a fused ring with R¹. In this embodiment the substituent T on R¹ forms a fused ring with the carbon atom adjacent to the carbon atom to which it is attached.

Examples of compounds where T forms a fused ring with R¹ include:

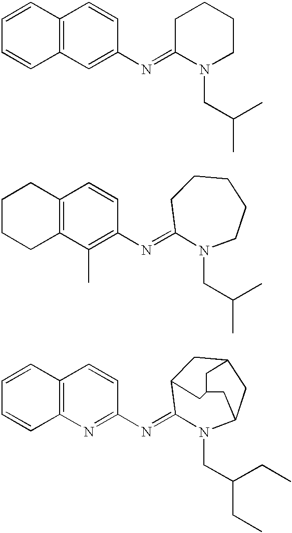

In still other embodiments of the compounds of the general formula I, the number of substituents T on R¹ (t) is between 2 and 5 when R¹⁰, R¹¹ and R¹² all are hydrogen. Furthermore, in preferred embodiments of the invention, at least one of T is nitro, nitrile, halogen or haloalkyl.

In preferred embodiments of the compounds of the general formula II:

R¹³ is aryl of 6-12 carbon atoms and is more preferably phenyl;

R¹⁴ is alkyl of 2-10 carbon atoms or cycloalkyl of 3-12 carbon atoms;

T′ is a substituent on R¹³ and is selected from nitro, nitrile, trifluoromethyl and halogen;

t′ is the number of substituents T′ and is between 1 and 3; and

R¹⁵ and R¹⁶ are either independently selected from alkyl of 2-10 carbon atoms and cycloalkyl of 3-6 carbon atoms or, together with the nitrogen atom to which they are attached, form a 5-8 membered ring of 4-7 carbon atoms and 1-2 heteroatoms, such as N, S and O.

For preferred compounds of formula II in which R¹⁵ and R¹⁶ are either alkyl of 2-10 carbon atoms or cycloalkyl of 3-6 carbon atoms, the sum of non-hydrogen atoms in R¹⁴, R¹⁵ and R¹⁶ is greater than or equal to 9. Examples of preferred compounds of formula II in which R¹⁵ and R¹⁶ are either alkyl of 2-10 carbon atoms or cycloalkyl of 3-6 carbon atoms include:

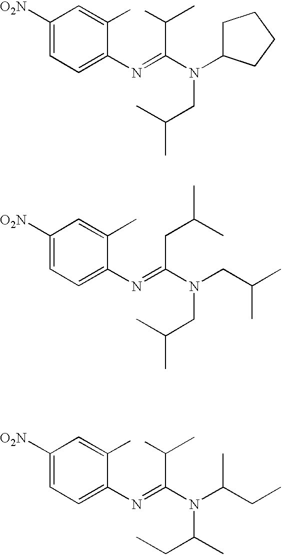

Examples of preferred compounds of formula II in which R¹⁵ and R¹⁶ joined to form a 5-8 membered ring together with the nitrogen atom to which they are attached include:

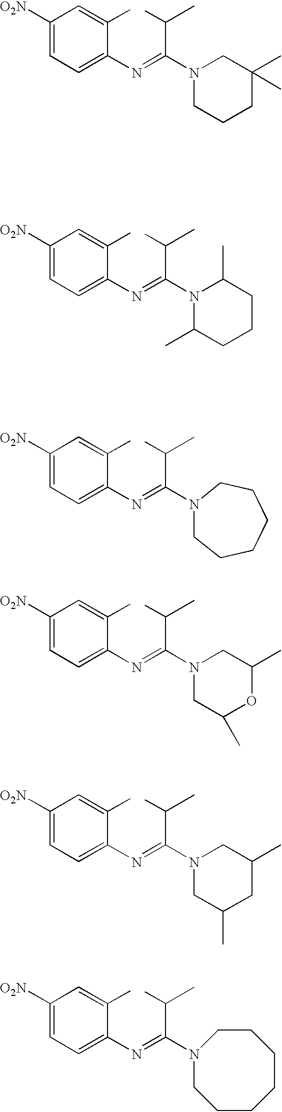

Compounds of formulas I-IV may be useful in the treatment or prevention of progesterone receptor mediated diseases or conditions. An agent which binds to the progesterone receptor may be employed for a wide variety of indications, including those shown in the lettered paragraphs below:

A1) to enhance bone formation in bone weakening diseases, for the prevention of and/or treatment of osteopenia or osteoporosis (Manzi, et al., J. Soc. Gynecol. Invest., 1, 302 (1994); Scheven, et al., Biochem. Biophys. Res. Commun., 186, 54 (1992); Verhaar, et al., Bone, 15, 307 (1994); Ontjes, In “Calcium and Phosphorus in Health Diseases”, Anderson and Garner (Eds.), CRC Press, 207 (1996); Scheven et al., Biochem. Biophys. Res. Commun., 186, 54 (1992)) including corticosteroid-induced osteoporosis (Picardo, et al., Drug Safety 15, 347 (1996)), postmenopausal osteoporosis, or Paget's disease;

A2) as an agent to enhance fracture healing;

B1) as a female contragestive agent, (Cadepond et al., Annu. Rev. Med., 48, 129 (1997); Heikinheimo Clin. Pharmacokinet., 33, 7 (1997); Li et al., Adv. Contracept., 11, 285 (1995); Spitz et al., Adv. Contracept. 8, 1 (1992); Spitz et al., Annu. Rev. Pharmacol. Toxicol., 36,47 (1996));

B2) for prevention of endometrial implantation (Cadepond et al., Annu. Rev. Mod., 48, 129 (1997));

B3) for the induction of labor (Heikinheimo Clin. Pharmacokinet., 33, 7 (1997); Karalis et al., Ann. N. Y. Acad. Sci., 771, 551 (1995)), including the case of foetus mortus (Heikinheimo, Clin. Pharmacokinet., 33, 7 (1997); Cadepond et al., Annu. Rev. Med., 48, 129 (1997));

B4) for treatment of luteal deficiency (Pretzsh et al., Zentralbl. Gynaekol., 119 (Suppl. 2), 25 (1997); Bezer et al., In “Molecular and Cellular Aspects of Periimplantation Processes”, Dey (Ed.), Springer-Verlag, p. 27 (1995));

B5) to enhance recognition and maintanence of pregnancy (Bezer et al., In “Molecular and Cellular Aspects of Periimplantation Processes”, Dey (Ed.), Springer-Verlag, p. 27 (1995));

B6) for counteracting preeclampsia, eclampsia of pregnancy and preterm labor (Yallampalli et al., WO 97/34,922);

B7) for the treatment of infertility, including promotion of spermatogenesis, the induction of the acrosome reaction, oocyte maturation, and in vitro fertilization of oocytes (Baldi et al., J. Steroid Biochem. Mol. Biol., 53 199 (1995); Baldi et al., Trends Endocrinol. Metab., 6, 198 (1995); Blackwell et al., Colloq. INSERM, 236, 165 (1995); Blackmore et al., Cell. Signalling, 5, 531 (1993); Cork et al., Zygote, 2, 289 (1994); Meizel, Biol. Reprod., 56, 569 (1997));

C1) for treatment of dysmenorrhea (Coll Capdevila et al., Eur. J. Contracept. Reprod. Health Care, 2, 229 (1997); Adashi et al., Keio J. Med., 44, 124 (1995));

C2) for treatment of dysfunctional uterine bleeding (Coll Capdevila et al., Eur. J. Contracept. Reprod. Health Care, 2, 229 (1997); Adashi et al., Keio J. Med., 44, 124 (1995));

C3) for treatment of ovarian hyperandrogynism (Schaison et al., Androg. Excess Disord. Women, 715 (1997));

C4) for treatment of ovarian hyperaldosteronism (Adashi et al., Keio J. Med., 44, 124 (1995));

C5) for treatment of premenstral syndrome and/or premenstral tension (ortola, Curr. Opin. Endocrinol. Diabetes, 2, 483 (1995)); Adashi et al., Keio J. Med., 44, 124 (1995));

C6) for treatment of perimenstrual behavior disorders (Constant et al., Hormone Res., 40, 141 (1993));

C7) for treatment of climeracteric disturbance, i.e. menopause transition (Adashi et al., Keio J. Med., 44, 124 (1995)) including hot flushes (Sarrel, Int. J. Fertil. Women's Med., 42, 78 (1997); Bäckström et al., Ciba Found. Symp., 121, 171 (1995)), mood changes (Bäckström et al., Ciba Found. Symp., 121, 171 (1995)), sleep disturbance (Sarrel, Int. J. Fertil. Women's Med., 42, 78 (1997)) and vaginal dryness (Sarrel, Int. J. Fertil. Women's Med., 42, 78 (1997));

C8) for enhancement of female sexual receptivity (Dei et al., Eur. J. Contracept Reprod. Health Care, 2(4), 253 (1997); McCarthy et al., Trends Endocrinol. Metab., 7, 327-333 (1996); Mani et al., Horm. Behav., 31, 244 (1997)) and male sexual receptivity (Johnson et al., In “Essential Reproduction, 2^{nd} ed., Blackwell Scientific Pub., London p177 (1984));

C9) for treatment of post menopausal urinary incontinence (Mäkinen et al., Maturitas, 22, 233 (1995); Batra et al., J. Urology, 138, 1301 (1987));

C10) to improve sensory and motor functions (Bäckström et al., Ciba Found. Symp., 121, 171 (1995));

C11) to improve short term memory (Backstrom et al., Ciba Found. Symp., 121, 171 (1995));

C12) for treatment of postpartum depression (Dalton, Practitioner, 229, 507 (1985));

C13) for treatment of genital atrophy (Sarrel, Int. J. Fertil. Women's Med., 42, 78 (1997));

C14) for prevention of postsurgical adhesion formation (Ustun, Gynecol. Obstet. Invest., 46, 202 (1998));

C15) for regulation of uterine immune function (Hansen et al., J. Reprod. Fertil., 49(Suppl.), 69 (1995));

C16) for prevention of myocardial infarction (Sarrel, Int. J. Fertil. Women's Med., 42 78 (1997));

D1) for hormone replacement therapy (Casper et al., J. Soc. Gynecol. Invest., 3, 225 (1996));

E1) for treatment of cancers, including hormone mediated cancers, such as breast cancer (Cadepond et al., Annu. Rev. Med., 48, 129 (1997); Pike et al., Endocr.-Relat. Cancer, 4, 125 (1997)), uterine cancer (Heikinheimo Clin. Pharmacokinet., 33, 7 (1997)), ovarian cancer (Pike et al., Endocr.-Relat. Cancer, 4, 125 (1997); Hughes, WO 98/10,771), and endometrial cancer (Satyaswaroop, Contrib. Oncol., 50, 258 (1995); Pike et al., Endocr.-Relat. Cancer, 4, 125 (1997));

E2) for treatment of endometriosis (Cadepond et al., Annu. Rev. Med., 48, 129 (1997); Heikinheimo, Clin. Pharmacokinet., 33, 7 (1997); Edmonds, Br. J. Obstet. Gynaecol., 103 (Suppl. 14), 10 (1996); Adashi et al., Keio J. Med., 44, 124 (1995));

E3) for treatment of uterine fibroids (Cadepond et al., Annu. Rev. Med., 48, 129 (1997); Adashi et al., Keio J. Med., 44, 124 (1995));

F1) for treatment of hirsutism (Orentreich et al., U.S. Pat. No. 4,684,635; Azziz et al., J. Clin. Endocrinol. Metab., 80, 3406 (1995));

F2) for inhibition of hair growth (Houssay et al., Acta Physiol. Latinoam., 28, 11 (1978));

G1) as a male contraceptive (Hargreave et al., Int. Congr., Symp. Semin. Ser., 12, 99 (1997); Meriggiola et al., J. Androl., 18, 240 (1997));

G2) as an abortifacient (Michna et al., Pharm. Ztg., 141, 11 (1996)); and

H1) for the promotion of mylin repair (Baulieu et al., Cell. Mol. Neurobiol., 16, 143 (1996); Baulieu et al., Mult. Scler., 3, 105 (1997); Schumakei et al., Dev. Neurosci., 18, 6 (1996); Koenig et al., Science, 268, 1500 (1995)).

Compounds of formulas I-IV are preferably used in the treatment or prevention of osteopenia, osteoporosis, or bone fracture, or are used as female contragestive agents or as agents for hormone replacement.

Currently, progesterone or progestins alone or in combination with estrogens are clinically indicated: for contraception (Merck Manual; Merck & Co. (1992)); for treatment of gastrointestional bleeding due to arteriovenous malformations (Merck Manual; Merck & Co. (1992)); for treatment of recurrent metatarsal stress fractures complicated by oligiomenorrhea or amenorrhea (Merck Manual; Merck & Co. (1992)); for treatment of premenstral syndrome (PMS, premenstral tension; Merck Manual; Merck & Co. (1992)); for postmenopausal hormone replacement therapy (Merck Manual; Merck & Co. (1992)); for treatment of hot flashes and subsequent insomnia and fatigue during menopause (Merck Manual; Merck & Co. (1992)); for treatment of dysfunctional uterine bleeding when pregnancy is not desired (Merck Manual; Merck & Co. (1992)); and for suppression of endometriosis (Merck Manual; Merck & Co. (1992)), breast cancer (Merck Manual; Merck & Co. (1992)), endometrial cancer Merck Manual; Merck & Co. (1992)), or luteal insufficiency (Merck Manual; Merck & Co. (1992)). For example, medroxyprogesterone, a progestin, alone or in combination with estrogens is indicated for prevention of osteoporosis, treatment of vulvar and/or vaginal atrophy, treatment of moderate to severe vasomotor symptoms associated with menopause, treatment of secondary amenorrehea, treatment of abnormal uterine bleeding due to hormonal imbalance in the absence of organic pathology, prevention of pregnancy, or as adjunctive therapy and palliative treatment of inoperable, recurrent, and metastatic endometrial or renal carcinoma (Merck Manual; Merck & Co. (1998)).

The present invention also includes pharmaceutically acceptable salts of the compounds of Formulas I-IV. Suitable pharmaceutically acceptable salts are well known to those skilled in the art and include basic salts of inorganic and organic acids, such as hydrochloric acid, hydrobromic acid, sulphuric acid, phosphoric acid, methanesulphonic acid, trifluoromethanesulfonic acid, sulphonic acid, acetic acid, trifluoroacetic acid, malic acid, tartaric acid, citric acid, lactic acid, oxalic acid, succinic acid, fumaric acid, maleic acid, benzoic acid, salicylic acid, phenylacetic acid, and mandelic acid. In addition, pharmaceutically acceptable salts include acid salts of inorganic bases, such as salts containing alkaline cations (e.g., Li⁺Na⁺or K⁺), alkaline earth cations (e.g., Mg⁺², Ca⁺² or Ba⁺²), the ammonium cation, as well as acid salts of organic bases, including aliphatic and aromatic substituted ammonium, and quaternary ammonium cations such as those arising from protonation or peralkylation of triethylamine, N,N-diethylamine, N,N-dicyclohexylamine, pyridine, N,N-dimethylaminopyridine (DMAP), 1,4-diazabicyclo[2.2.2]octane (DABCO), 1,5-diazabicyclo[4.3.0]non-5-ene (DBN) and 1,8-diazabicyclo[5.4.0]undec-7-ene (DBU).

A number of the compounds of Formulas I-IV possess asymmetric carbons and can therefore exist in racemic and optically active forms. Methods of separation of enantiomeric and diastereomeric mixtures are well known to the skilled in the art. The present invention encompasses any racemic or optically active forms of compounds described in Formula I or Formula II which possess progesterone receptor binding activity or the use of any racemic or optically active forms of compounds described in Formulas I-IV for the treatment or prevention of progesterone receptor mediated diseases or conditions.

The therapeutic agents of the invention may be employed alone or concurrently with other therapies. For example, when employed as in A1 or A2, the agent may be used in combination with a calcium source, vitamin D or analogues of vitamin D, and/or antiresorptive therapies such as estrogen replacement therapy, treatment with a fluoride source, treatment with calcitonin or a calcitonin analogue, or treatment with a bisphosphonate such as alendronate. When employed as in B1 through B7, the agent may be used with therapies such as estrogen replacement therapy. When employed as in C1 through C16, E1 through E3, or F1 or F2, the agent may be used concurrently with therapies such as estrogen replacement therapy and/or a gonadotropin-releasing hormone agonist. When employed as in G1 or G2, the agent maybe used concurrently with therapies such as an androgen.

The method of the invention is intended to be employed for treatment of progesterone receptor mediated diseases or conditions in both humans and other mammals.

The compounds may be administered orally, dermally, parenterally, by injection, by inhalation or spray, or sublingually, rectally or vaginally in dosage unit formulations. The term ‘administered by injection’ includes intravenous, intraarticular, intramuscular, subcutaneous and parenteral injections, as well as use of infusion techniques. Dermal administration may include topical application or transdermal administration. One or more compounds may be present in association with one or more non-toxic pharmaceutically acceptable carriers and, if desired, other active ingredients.

Compositions intended for oral use may be prepared according to any suitable method known to the art for the manufacture of pharmaceutical compositions. Such compositions may contain one or more agents selected from the group consisting of diluents, sweetening agents, flavoring agents, coloring agents and preserving agents in order to provide palatable preparations.

Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; and binding agents, for example magnesium stearate, stearic acid or talc. The tablets may be uncoated or they may be coated by known techniques to delay disintegration and adsorption in the gastrointestinal tract and thereby provide a sustained action over a longer period. For example, a time delay material such as glyceryl monostearate or glyceryl distearate may be employed. These compounds may also be prepared in solid, rapidly released form.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions containing the active materials in admixture with excipients suitable for the manufacture of aqueous suspensions may also be used. Such excipients are suspending agents, for example sodium carboxymethylcellulose, methylcellulose, hydroxypropyl-methylcellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl,p-hydroxybenzoate, one or more coloring agents, one or more flavoring agents, and one or more sweetening agents, such as sucrose or saccharin.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example, sweetening, flavoring and coloring agents, may also be present.

The compounds may also be in the form of non-aqueous liquid formulations, e.g., oily suspensions which may be formulated by suspending the active ingredients in a vegetable oil, for example arachis oil, olive oil, sesame oil or peanut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide palatable oral preparations. These compositions may be preserved by the addition of an anti-oxidant such as ascorbic acid.

Pharmaceutical compositions of the invention may also be in the form of oil-in-water emulsions. The oil phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soybean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The compounds may also be administered in the form of suppositories for rectal or vaginal administration of the drug. These compositions can be prepared by mixing the drug with a suitable non-irritating excipient which is solid at ordinary temperatures but liquid at the rectal or vaginal temperature and will therefore melt in the rectum or vagina to release the drug. Such materials include cocoa butter and polyethylene glycols.

Compounds of the invention may also be administered transdermally using methods known to those skilled in the art (see, for example: Chien; “Transdermal Controlled Systemic Medications”; Marcel Dekker, Inc.; 1987. Lipp et al. WO 94/04157 Mar. 3, 1994). For example, a solution or suspension of a compound of Formula I or II in a suitable volatile solvent optionally containing penetration enhancing agents can be combined with additional additives known to those skilled in the art, such as matrix materials and bacteriocides. After sterilization, the resulting mixture can be formulated following known procedures into dosage forms. In addition, on treatment with emulsifying agents and water, a solution or suspension of a compound of Formula I or II may be formulated into a lotion or salve.

Suitable solvents for processing transdermal delivery systems are known to those skilled in the art, and include lower alcohols such as ethanol or isopropyl alcohol, lower ketones such as acetone, lower carboxylic acid esters such as ethyl acetate, polar ethers such as tetrahydrofuran, lower hydrocarbons such as hexane, cyclohexane or benzene, or halogenated hydrocarbons such as dichloromethane, chloroform, trichlorotrifluoroethane, or trichlorofluoroethane. Suitable solvents may also include mixtures one or more materials selected from lower alcohols, lower ketones, lower carboxylic acid esters, polar ethers, lower hydrocarbons, halogenated hydrocarbons.

Suitable penetration enhancing materials for transdermal delivery systems are known to those skilled in the art, and include, for example, monohydroxy or polyhydroxy alcohols such as ethanol, propylene glycol or benzyl alcohol, saturated or unsaturated C₈-C₁₈ fatty alcohols such as lauryl alcohol or cetyl alcohol, saturated or unsaturated C₈-C₁₈ fatty acids such as stearic acid, saturated or unsaturated fatty esters with up to 24 carbons such as methyl, ethyl, propyl, isopropyl, n-butyl, sec-butyl isobutyl tert-butyl or monoglycerin esters of acetic acid, capronic acid, lauric acid, myristinic acid, stearic acid, or palmitic acid, or diesters of saturated or unsaturated dicarboxylic acids with a total of up to 24 carbons such as diisopropyl adipate, diisobutyl adipate, diisopropyl sebacate, diisopropyl maleate, or diisopropyl fumarate. Additional penetration enhancing materials include phosphatidyl derivatives such as lecithin or cephalin, terpenes, amides, ketones, ureas and their derivatives, and ethers such as dimethyl isosorbid and diethyleneglycol monoethyl ether. Suitable penetration enhancing formulations may also include mixtures one or more materials selected from monohydroxy or polyhydroxy alcohols, saturated or unsaturated C₈-C₁₈ fatty alcohols, saturated or unsaturated C₈-C₁₈ fatty acids, saturated or unsaturated fatty esters with up to 24 carbons, diesters of saturated or unsaturated dicarboxylic acids with a total of up to 24 carbons, phosphatidyl derivatives, terpenes, amides, ketones, ureas and their derivatives, and ethers.

Suitable binding materials for transdermal delivery systems are known to those skilled in the art and include polyacrylates, silicones, polyurethanes, block polymers, styrene-butadiene coploymers, and natural and synthetic rubbers. Cellulose ethers, derivatized polyethylenes, and silicates may also be used as matrix components. Additional additives, such as viscous resins or oils may be added to increase the viscosity of the matrix.

For all regimens of use disclosed herein for compounds of Formula I-IV, the daily oral dosage regimen will preferably be from 0.01 to 200 mg/Kg of total body weight. The daily dosage for administration by injection, including intravenous, intramuscular, subcutaneous and parenteral injections, and use of infusion techniques will preferably be from 0.01 to 200 mg/Kg of total body weight. The daily rectal dosage regimen will preferably be from 0.01 to 200 mg/Kg of total body weight. The daily vaginal dosage regimen will preferably be from 0.01 to 200 mg/Kg of total body weight. The daily topical dosage regimen will preferably be from 0.1 to 200 mg administered between one to four times daily. The transdermal concentration will preferably be that required to maintain a daily dose of from 0.01 to 200 mg/Kg. The daily inhalation dosage regimen will preferably be from 0.01 to 10 mg/Kg of total body weight.

It will be appreciated by those skilled in the art that the particular method of administration will depend on a variety of factors, all of which are considered routinely when administering therapeutics. It will also be understood, however, that the specific dose level for any given patient will depend upon a variety of factors, including, but not limited to the activity of the specific compound employed, the age of the patient, the body weight of the patient, the general health of the patient, the gender of the patient, the diet of the patient, time of administration, route of administration, rate of excretion, drug combinations, and the severity of the condition undergoing therapy. It will be further appreciated by one skilled in the art that the optimal course of treatment, ie., the mode of treatment and the daily number of doses of a compound of Formula I-IV or a pharmaceutically acceptable salt thereof given for a defined number of days, can be ascertained by those skilled in the art using conventional treatment tests.

The entire disclosures of all applications, patents and publications cited above and below are hereby incorporated by reference.

The compounds of Formulas I-IV may be prepared by use of known chemical reactions and procedures, from known compounds (or from starting materials which, in turn, are producible from known compounds) through the preparative methods shown below as well as by other reactions and procedures known to the skilled in the art. Nevertheless, the following general preparative methods are presented to aid practitioners in synthesizing the compounds of the invention, with more detailed particular examples being presented in the experimental section. The examples are for illustrative purposes only and are not intended, nor should they be construed, to limit the invention in any way.

The invention generally pertains to compounds of formula V.

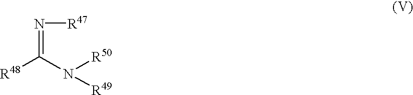

wherein

R⁴⁷ is R¹(T)ₜ, R²⁵(Q)_{q}, R¹³(T′)_{t′}, or R³⁵(Q′)_{q′}; R⁵⁰ is R² (G)_{g}, R²⁶(G′)_{g′}, R¹⁶ or R³⁸; R⁴⁹ and R⁴⁸ are R¹⁵ and R¹⁴ respectively, R³⁷ and R³⁶ respectively, or R⁴⁹ and R⁴⁸ may be joined to form a linking group —X— or —X′—; and R¹, R², R¹³, R¹⁴, R¹⁵, R¹⁶, R²⁵, R²⁶, R³⁵, R³⁶, R³⁷, R³⁸, T, T′, G, G′, Q, Q′, t, t′, g, g′, q and q′ are as defined hereinabove for formulas I-IV.

Formula V amidines are prepared by straightforward methods known to those in the art. Methods that are illustrative of those which may be used are described below and are not meant to be comprehensive or limiting in any way. Such methods may be used in the preparation of the cyclic or acyclic amidines of formulas I-IV. For example, the amidines of Formula V may be prepared in the manner shown in Flow Diagram I.

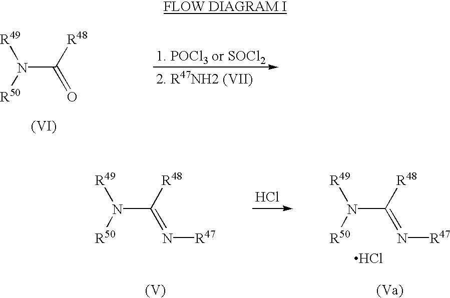

An amide of formula VI may be treated, at a temperature from −78° to 20° C., with an agent, such as phosphorous oxychloride or thionyl chloride, neat or in an inert solvent such as methylene chloride and with or without addition of a catalytic amount of dimethylformamide, to form an intermediate chloroiminium chloride or imidoyl chloride. This intermediate is not isolated but is further treated, after removal of any excess phosphorous oxychloride or thionyl chloride, with an amino compound of formula VII and a base, such as triethylamine. The amidine product of formula V is isolated upon aqueous workup, and may be further derivatized to an acid salt Va, by treatment with an anhydrous acid such as gaseous HCl in an anhydrous solvent such as ether.

Alternatively, an amide of formula VIII may be treated sequentially with an activating agent, such as thionyl chloride and the like, and an amine to provide formula V compounds, as shown in Flow Diagram II:

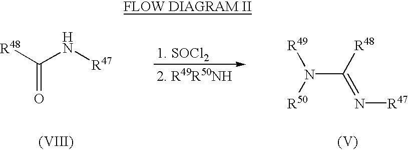

Formula I compounds in which R⁵⁰ is a hydrogen may be converted to formula I compounds where R⁵⁰ is an alkyl group. This reaction sequence is illustrated in Flow Diagram III, in which the amidine is first treated with a base such as sodium hydride or cesium carbonate in an anhydrous aprotic solvent, such as N,N-dimethylformamide (DMF), and then allowed to react with an alkyl halide such as methyl iodide, sec-butyl bromide or the like.

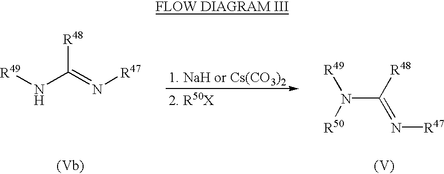

An additional route to formula V compounds is shown in Flow Diagram TV. By this method, a urea of formula IX is reacted with an excess of an alkyl, alkenyl or alkynyl magnesium halide Grignard reagent at room temperature in an aprotic solvent such as THF to provide corresponding formula V compounds as shown:

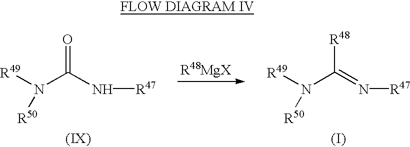

Certain formula V compounds in which R⁴⁹ or R⁵⁰ contain a terminal double bond may be converted to isomeric formula V compounds by treatment with an acidic reagent such as polyphosphoric acid (PPA) from room temperature to 90° C. exemplified in Flow Diagram V.

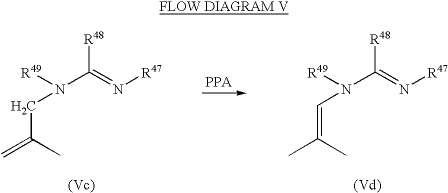

Amides of formula VI may be prepared by methods well know to those skilled in the art, for instance by acylation of a mono or disubstituted amine with an acylating agent such as an acid chloride, with or without the addition of a base catalyst, such as triethylamine, as shown in Flow Diagram VI.

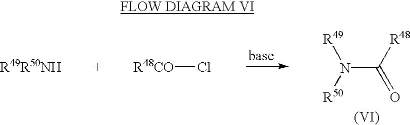

Certain formula VI compounds in which R⁴⁸and R⁴⁹ are joined to form a linking group —X— or —X′—, as described above, may be prepared by thermal intramolecular cyclization of an amino ester derivative of formula X, where R¹ is lower alkyl, as shown in Flow Diagram VII.

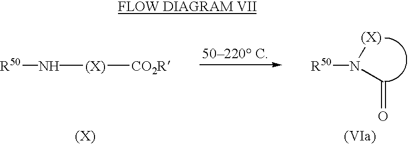

Formula VI compounds may also be prepared by alkylation of disubstituted formula VI compounds with a suitable alkylation agent such as an alkyl halide, in the presence of a base such as sodium hydride, in the presence or absence of an aprotic solvent, such as DMF, as shown in Flow Diagram VIII.

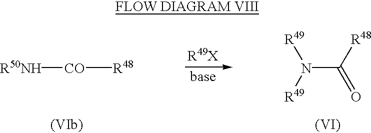

Formula VIb compounds are either commercially available or may generally be prepared by acylation of an unsubstituted amine with an acylating agent. In some cases, compounds of formula VIb may be prepared from a ketone of formula IX by the sequence shown in Flow Diagram IX. The ketone is converted to an oxime, then induced to undergo rearrangement to VIb, catalyzed, for example, by an optionally substituted benzene sulfonyl chloride. Formula IX ketones are readily available from a wide variety of commercial sources and may be prepared by well-known routes, such as oxidation of the corresponding alcohols. Formula DC ketones may also be prepared by rearrangement reactions such as pinacol/pinacolone type rearrangements of 1,2-diols, as shown for the preparation of IXa from XI, and the acid-catalyzed rearrangement of epoxides.

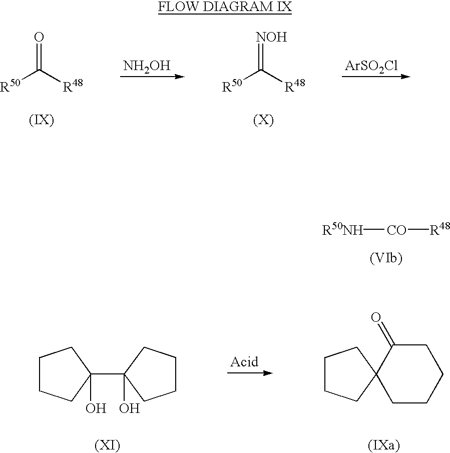

Compounds of formula VIII may be prepared by reaction of an amino compound of formula VII with an acylating agent such as an acid halide, at 0° C. to 25° C. with the addition of a base such as triethylamine.

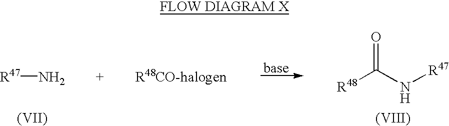

### Preparative Examples

The following examples are included as illustrations of preparation of specific compounds of the invention, and are not to be construed as limiting the scope of the invention in any way.

The compounds useful in the therapeutic method of this invention are prepared by standard methods of organic chemistry, prepared individually or by parallel synthesis. Unless otherwise noted reagents and solvents were obtained from commercial suppliers and were used without further purification.

All reactions were carried out in flaine-dried or oven-dried glassware under a positive pressure of dry argon or dry nitrogen, and were stirred magnetically unless otherwise indicated. Sensitive liquids and solutions were transferred via syringe or cannula, and introduced into reaction vessels through rubber septa. Commercial grade reagents and solvents were used without further purification.

Unless otherwise stated, the term “concentration in vacuo” refers to use of a Buchi rotary evaporator at approximately 15 mmHg. Bulb-to-bulb concentrations were conducted using an Aldrich Kugelrohr apparatus, and in these cases temperatures refer to oven temperatures. All temperatures are reported uncorrected in degrees Celsius (° C.). Unless otherwise indicated, all parts and percentages are by volume.

Thin-Layer chromatography (TLC) was performed on Whatman® pre-coated glass-backed silica gel 60A F-254 250 μm plates. Visualization of plates was effected by one or more of the following techniques: (a) ultraviolet illumination, (b) exposure to iodine vapor, (c) immersion of the plate in a 10% solution of phosphomolybdic acid in ethanol followed by heating, (d) immersion of the plate in a cerium sulfate solution followed by heating, and/or (e) immersion of the plate in an acidic ethanol solution of 2,4-dinitrophenylhydrazine followed by heating. Column chromatography (chromatography) was performed using 230-400 mesh EM Science® silica gel. Rotary chromatography was performed using pre-cast SiO₂ plates (Alltech®) from Harrison Research Chromatotron.

Melting points (mp) were determined using a Thomas-Hoover melting point apparatus or a Mettler FP66 automated melting point apparatus and are uncorrected.

Fourier transform infrared spectra were obtained using a Mattson 4020 Galaxy Series spectrophotometer.

Proton (¹H) nuclear magnetic resonance (NM) spectra were measured with a General Electric GN-Omega 300 (300 MHz) spectrometer with either Me₄Si (δ0.00) or residual protonated solvent (CHCl_{3 δ}7.26; MeOH δ3.30; DMS m/z,O δ2.49) as standard. Carbon (¹³C) NMR spectra were measured with a General Electric GN-Omega 300 (75 MHz) spectrometer with solvent (CDCI δ77.0; MeOD-d₃; δ49.0; DMSO-d₆ O 39.5) as standard.

Low resolution mass spectra (MS) and high resolution mass spectra (HRMS) were obtained as electron impact (El), chemical ionization (CI), or as fast atom bombardment (FAB) mass spectra. Electron impact mass spectra (El-MS) were obtained with a Hewlett Packard 5989A mass spectrometer equipped with a Vacumetrics Desorption Chemical Ionization Probe for sample introduction. The ion source was maintained at 250° C. Electron impact ionization was performed with electron energy of 70 e V and a trap current of 300 μA. Liquid-cesium secondary ion mass spectra (FAB-MS), an updated version of fast atom bombardment, were obtained using a Kratos Concept I-H spectrometer. Chemical ionization mass spectra (CI-MS) were obtained using a Hewlett Packard MS-Engine (5989A) with methane or ammonia as the reagent gas (1×10⁻⁴ torr to 2.5×I0⁻⁴ torr). The direct insertion de sorption chemical ionization (DCI) probe (Vacumetrics, Inc.) was ramped from 0-1.5 amps in 10 sec and held at 10 amps until all traces of the sample disappeared (about 1-2 min). Spectra were scanned from 50-800 amu at 2 see per scan. HPLC-electrospray mass spectra (HPLC ES-MS) were obtained using a Hewlett-Packard 1100 HPLC equipped with a quaternary pump, a variable wavelength detector, a C-18 column, and a Finnigan LCQ ion trap mass spectrometer with electrospray ionization. Spectra were scanned from 120-800 amu using a variable ion time according to the number of ions in the source. Gas chromatography-ion selective mass spectra (GC-MS) were obtained with a Hewlett Packard 5890 gas chromatograph equipped with an HP-1 methyl silicone column (0.33 mM coating; 25 m×0.2 mm) and a Hewlett Packard 5971 Mass Selective Detector (ionization energy 70 eV).

Elemental analyses were conducted by Robertson Microlit Labs, Madison N.J. NMR spectra, LRMS, elemental analyses, and HRMS of the compounds were consistent with the assigned structures.

The IUPAC names of compounds exemplified were obtained using the ACD/Lab Web Service.

Examples of preparations of compounds of the invention are provided in the following detailed synthetic procedures.

### EXAMPLE 1

### Preparation of 3-(1-Nitrocyclopentyl)-propionic acid, Methyl Ester

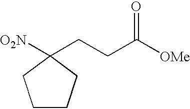

A solution of nitropentane (4.50 g; 39.1 mmol), dioxane (2 mL) and 40 wt % Triton B (0.4 mL, 0.9 mmol) was warmed to 70° C., and after stirring for 5 minutes, methyl acrylate (3.5 mL, 39.0 mmol) was added dropwise over 15 minutes. The reaction mixture was stirred for an additional 2.5 h at 70° C. and then cooled to ambient temperature. The reaction was partitioned between Et₂O and 1N HCl. The separated organic layer was washed successively with water(twice), 0.1% sodium bicarbonate solution, dried (Na₂SO₄), filtered and concentrated in vacuo to yield the desired compound as a greenish oil (7.95 g, 39 mmol, 100%) which was used in the next step without further purification. GCMS m/z, 155 [M-NO₂]⁺.

### EXAMPLE 2

### Preparation of 3-(1-Nitrocyclopentyl)-propionic Acid

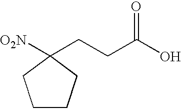

To a solution of 3-(1-nitrocyclopentyl)propionic acid, methyl ester (8.45 g, 42 mmol) in THF (200 mL) at 0° C. was added aqueous 1N NaOH (46 mL). The mixture was stirred 10 minutes then warmed to ambient temperature and stirred for 2.5 h. The mixture was then concentrated in vacuo to about ¼ volume and was partitioned between Et₂O and water. The aqueous layer was washed with Et₂O, the pH was adjusted to 0 with 1N HCl and then extracted with Et₂O. The Et₂O extracts were combined and dried (MgSO₄), then filtered and concentrated to yield the product as a light yellow oil (7.60 g, 40.6 mmol, 97%) which was used in following steps without further purification.

### EXAMPLE 3

### Preparation of 1-Diazo-4-(1-nitrocyclopentyl)-2-butanone

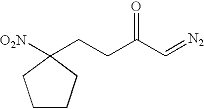

To a solution of 3-(1-nitrocyclopentyl)-propionic acid (1.90 g, 10.2 mmol) in dry CH₂Cl₂ was added thionyl chloride (0.81 mL, 11.2 mmol) and 3 drops DMF. The reaction was gently warmed to 35° C. and stirred for 2.5 h. The reaction mixture was concentrated in vacuo and suspended in CH₂Cl₂ (20 mL) and cooled to −10° C. Diazomethane was added until a yellow color persisted, and the reaction was stoppered and allowed to warm slowly to ambient temperature with stirring for an additional 12 h. The reaction mixture was concentrated to an orange oil (2.51 g), which was used in following steps without further purification. TLC (hexane-EtOAc, 9:1), R_{f}=0.22.

### EXAMPLE 4

### Preparation of 4-(1-Nitrocyclopentyl)-butanoic Acid, Methyl Ester

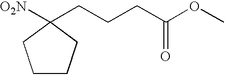

A filtered solution of silver benzoate(500 mg) and triethylamine (5 mL) was prepared and added in 0.5 mL portions to a solution of 1-diazo-4-(1-nitrocyclopentyl)-2-butanone (2.15 g, 10.2 mnnol) in MeOH at reflux until nitrogen evolution had ceased (about 2.5 mL total). The solution was cooled to ambient temperature, Celite® was added, the solution was filtered and then concentrated in vacuo. The residue was dissolved in Et₂O and was washed successively with 1N HCl, saturated sodium bicarbonate solution, saturated NaCl, and dried (MgSO₄). The solution was filtered and concentrated in vacuo and the crude material was chromatographed on silica gel, eluting with hexane-EtOAc mixtures to yield the desired compound as an oil (1.71 g, 7.96 mmol). TLC (hexane-EtOAc, 9:1), R_{f}=0.46.

### EXAMPLE 5

### Preparation of Spiro[4.5]decan-6-one

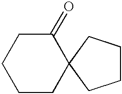

To a solution of(1,1′-bicyclopentyl)-1,1′-diol (3.0 g, 17.62 mmol) in 50 mL of CH2Cl2 was added 1.0 g of anhydrous (MgSO₄) and this suspension was stirred for 1 h. The reaction mixture was cooled in an ice bath, treated with 0.5 mL of boron trifluoride etherate and stirred for an additional h. Na₂CO₃ (2.5 g) was then added and stirring under argon was continued until the ice bath had melted and reached room temperature. The mixture was filtered and concentrated in vacuo, leaving a residue that was purified by chromatography (5% EtOAc/hexanes) to afford product (1.87 g, 70%). TLC (40% EtOAc/hexane), R_{f}=0.95.

### EXAMPLE 6

### Preparation of Spiro[4.5]decan-6-one Oxime

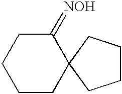

To a solution of 1.50 g (9.85 mmol) of spiro[4.5]decan-6-one in 80 mL MeOH was added 1.40 g (19.71 mmol) hydroxylamine hydrochloride followed by 2.40 g (29.6 mmol) NaOAc. The reaction mixture was stirred for 16 h at room temperature. The MeOH was then concentrated in vacuo leaving a white solid to which was added 80 mL CH₂Cl₂. The CH₂C₁₂ solution was washed with 80 mL of water, separated and dried (Na2SO4). Filtration followed by concentration in vacuo left a thick clear residue that crystallized on standing (1.55 g, 94%). TLC (40% EtOAc/hexanes), R_{f}=0.66.

### EXAMPLE 7

### Preparation of N,N,-diisobutyl-N′-(2-methyl-4-nitrophenyl)urea

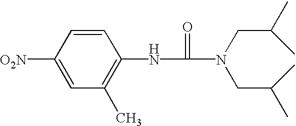

A solution of 20% phosgene (6.8 mL, 3.14 mmol) in toluene and CH₂Cl₂ (100 mL) was slowly treated at 0° C. with a solution of 2-methyl-4-nitroaniline (1.00 g, 6.57 mmol) and pyridine (0.97 mL, 19.7 mmol) in CH₂Cl₂ (100 mL). The mixture was stirred at room temperature for 2 h and was then concentrated in vacuo to a yellow residue. The residue was suspended in CH₂Cl₂ and treated with diisobutylamine (1.3 mL, 7.23 mmol). Stirring was continued for 18 h at room temperature. The mixture was washed with saturated aqueous sodium bicarbonate solution, dried (MgSO₄), filtered and concentrated in vacuo to a yellow oil. This material was triturated with Et₂O and the solids were collected by filtration (1.54 g, 77%). MS (FAB) m/z, 308 [M+H]⁺.

### EXAMPLE 8

### Preparation of N,N-Diisobutylacetamide

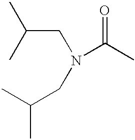

A solution of 1.56 g diisobutyl amine (12.06 mmol) in dry Et₂O (20 mL) was cooled in an ice water bath. Triethylamine (1.68 mL, 13.27 mmol) was added via syringe followed by dropwise addition of acetyl chloride (0.78 mL, 10.96 mmol) via syringe over a 5-minute period. Stiring was continued for 15 minutes, then the mixture was warmed to ambient temperature and stirring was continued for an additional 2.5 h. The reaction was diluted with Et₂O and washed with 1N HCl (three times), saturated NaCl. The organics were dried (MgSO₄), filtered and concentrated to yield clean desired compound as an oil (1.45 g, 8.48 mmol; 77% yield). TLC (hexanes-EtOAc, 2:1), R_{f}=0.38.

### EXAMPLE 9

### Preparation of 4-(2-Ethylbutyl)-4-azatricyclo[4.3.1.1 ^{3,8}]undecan-5-one

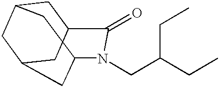

To a solution of 1.00 g, 6.05 mmol of 4-azatricyclo-[4.3.1.1^{3.8}]undecan-5-one in 50 mL of DMF was added 0.145 g (6.05 mmol) of sodium hydride. This was allowed to stir at room temperature for 1 h. To this mixture was then added 1.00 g (6.05 mmol) of 1 bromo-2-ethylbutane and it was heated at 110° C. for 16 h. The reaction mixture was then cooled and the DMF was concentrated in vacuo leaving a residue. This residue was taken up in CH₂Cl₂ and washed four times with 50 mL of water. The organic phase was separated, dried and concentrated in vacuo leaving an oil (0.51 g, 34%).

### EXAMPLE 10

### Preparation of 1-Aza-spiro[4.4]nonan-2-one

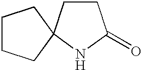

A mixture of 3-(1-nitrocyclopentyl)-propionic acid methyl ester (5.46 g, 27 mmol), absolute ethanol (100 mL) and 10% Pd/C (500 mg). was heated to 50° C. and to this solution hydrogen gas was introduced, displacing the argon, and the reaction was stirred for 2 days. To the reaction was added Celite® and the hot solution filtered and washed with hot MeOH. The filtrates were concentrated to about 50 mL and the resulting solid removed by filtration and vacuum dried to yield the desired product as a white solid (1.25 g, 9.0 mmol, 33%), suitable for use in the following steps without further purification.

### EXAMPLE 11

### Preparation of -2-Aza-2-isobutylbicyclo [2.2.2]-octan-3-one

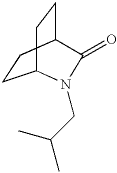

A dry, 100 mL sealed reaction flask was equipped with a stir bar. The flask was charged with methyl 4-isobutylaminocyclohexane carboxylate (5.88 g, 27.6 mmol) in dry MeOH (20 mL) and heated to 200° C. for 1.5 h. The mixture was allowed to cool and then concentrated in vacuo to yield the product (2.88 g, 15.9 mmol, 57%). MS (EI) m/z, 181 [M+H]⁺.

### EXAMPLE 12

### Preparation of 9-Azabicyclo [3.3.2]decan-10-one

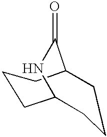

To a mixture of bicyclo[3.3.1]nonan-9-one (0.25 g, 1.81 mmol) in formic acid (20 mL) was added hydroxylamine sulfonic acid (0.31 g, 2.74 mmol) and stirring was continued for 18 h. The reaction was quenched using 1N NaOH (50 mL) and was extracted with twice with chloroform (50 mL). The organics were combined, dried (Na₂SO₄), filtered and concentrated to give the product (0.26 g, 1.69 mmol, 94%). TLC (hexanes-EtOAc, 5:1), R_{f}=0.05.

### EXAMPLE 13

### Preparation of 6-Azaspiro[4.6]undecan-7-one

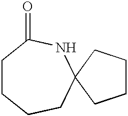

To a solution of 1.50 g (8.97 mmol) of spiro[4.5]decan-6-one in 30 mL acetone was added 1.65 g (1.2 mL, 9.36 mmol) benzenesulfonyl chloride. To this mixture was added 0.375 g (9.38 mmol of solid NaOH, followed by 7.5 mL of water. The reaction mixture was allowed to heat at 80° C. for 4 h. The reaction mixture was cooled and 50 mL water added. The acetone solution was concentrated in vacuo leaving an aqueous residue that was extracted with four 50-mL portions of Et₂O. The Et₂O extracts were combined and concentrated in vacuo leaving a viscous yellow oil. Trituration with hexanes afforded crystals that were filtered and dried (0.15 g, 10%). TLC (40% EtOAc/hexane) R_{f}=0.14.

### EXAMPLE 14

### Preparation of 4-(Isobutyl-4-azatricyclo[4.3.1.1^{3,8}]undecan-5-one

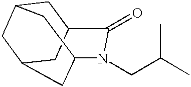

To a solution of 25.0 g (0.151 mmol) 4-azatricyclo[4.3.1.1^{3,8}]undecan-5-one in 200 mL 1-bromo-2-methylpropane was added with stirring 4.36 g (0.182) mmol sodium hydride and the mixture was allowed to heat at 80° C. for 17 h. The reaction mixture was then allowed to cool to room temperature and filtered. The 1-bromo-2-methylpropane was concentrated in vacuo leaving an oil, which was taken up in 150 mL of CH₂Cl₂ and washed with 100 mL of water. The organic phase was separated, dried (Na₂SO₄), filtered and concentrated in vacuo, leaving an oil (24.9 g, 74%).

### EXAMPLE 15

### Preparation of 4-(Cyclopentyl-4-azatricyclo[4.3.1.1^{3,8}]undecan-5-one

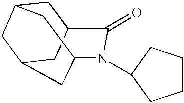

To a solution of 0.50 g (3.026 mmol) of 4-azatricyclo-[4.3.1.1^{3,8}]undecan-5-one in 20 mL cyclopentyl bromide was added 0.40 g( 1.51 mmol) 18-crown-6 followed by 0.29 g (12.1 mmol) sodium hydride. This reaction mixture was heated at 80° C. for 5 days, cooled, and the cyclopentyl bromide was removed in vacuo leaving a residue. The residue was taken up in CH₂Cl₂ and washed with water. The organic phase was separated, dried, filtered and concentrated in vacuo leaving an amber semi-solid (0.758 g, 107%).

Utilizing the procedures described in examples 8 to 15 above and employing the appropriate starting materials, examples 16-40 shown in Table 1 below were similarly prepared.

TABLE 1
Preparative Examples of Amides

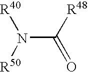

| Ex. | | | | Method of | |
|---|---|---|---|---|---|
| No. | R⁴⁹ | R⁵⁰ | R⁴⁸ | Example | Characterization |
| 8 | (CH₃)₂CH₂CH— | (CH₃)₂CH₂CH— | CH₃ | 8 | R_{f} = 0.38, 2:1 hexanes/EtOAc |
| 9 | (CH₃CH₂)₂CHCH_{2—} | 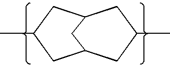 | | 9 | — |
| 10 | H | 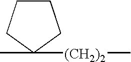 | | 10 | — |
| 11 | (CH₃)₂CHCH₂— | 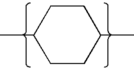 | | 11 | MS (EI) m/z, 181 [M + H]⁺ |
| 12 | H | 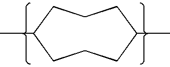 | | 12 | R_{f} = 0.05, 5:1 hexanes/EtOAc |
| 13 | H | 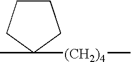 | | 13 | R_{f} = 0.14, 3:2 hexanes/EtOAc |
| 14 | (CH₃)₂CHCH_{2—} | 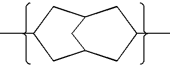 | | 14 | — |
| 15 | c-Pentyl | 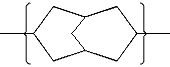 | | 15 | — |
| 16 | (CH₃)CH— | (CH₃)CH— | CH₃ | 8 | — |
| 17 | H | 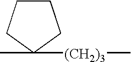 | | 9 | — |
| 18 | C₂H₅ | C₂H₅ | C₂H₅ | 9 | — |

| | | | | |
|---|---|---|---|---|
| 19 | H₂C═C(CH₃)CH₂— | 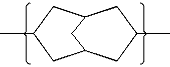 | 9 | R_{f} = 0.35, 2:3 EtOAc/hexanes |
| 20 | CH₃CH₂CH(CH₃)— | —(CH₂)₃— | 9 | R_{f} = 0.26, 2: EtOAc/hexanes |
| 21 | CH₃CH₂C(CH₃)CH₂— | 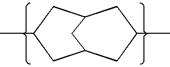 | 9 | R_{f} = 0.32, 2:3 EtOAc/hexanes |
| 22 | CH₃ | 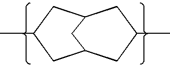 | 9 | R_{f} = 0.40, 2:3 EtOAc/hexanes |
| 23 | CH₃CH₂CH(CH₃)— | —(CH₂)₄— | 9 | R_{f} = 0.33, 2:3 EtOAc/hexanes |
| 24 | H₂C═C(CH₃)CH₂— | —(CH₂)₃— | 9 | R_{f} = 0.13, 2:3 EtOAc/hexanes |
| 25 | H₂C═C(CH₃)CH₂— | —(CH₂)₅— | 9 | R_{f} = 0.25, 2:3 EtOAc/hexanes |
| 26 | H₂C═C(CH₃)CH₂— | —(CH₂)₄— | 9 | R_{f} = 0.10, 2:3 EtOAc/hexanes |
| 27 | H₂C═C(CH₃)CH₂— | —(CH₂)₆— | 9 | R_{f} = 0.31, 2:3 EtOAc/hexanes |
| 28 | H₂C═C(CH₃)CH₂— | —(CH₂)₇— | 9 | R_{f} = 0.31, 2:3 EtOAc/hexanes |
| 29 | H₂C═C(CH₃)CH₂— | 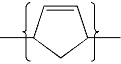 | 9 | R_{f} = 0.34, 2:3 EtOAc/hexanes |
| 30 | H₂C═C(CH₃)CH₂— | 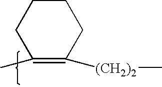 | 9 | R_{f} = 0.40, 2:3 EtOAc/hexanes |
| 31 | (CH₃CH₂)₂CHCH₂— | 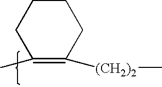 | 9 | R_{f} = 0.16, 2:3 EtOAc/hexanes |
| 32 | (CH₃CH₂)₂CHCH₂— | 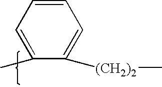 | 9 | R_{f} = 0.24, 2:3 EtOAc/hexanes |
| 33 | (CH₃CH₂)₂CHCH₂— | 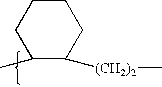 | 9 | R_{f} = 0.33, 2:3 EtOAc/hexanes |
| 34 | (CH₃)₂CHCH₂— | 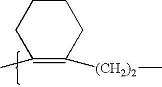 | 9 | R_{f} = 0.24, 2:3 EtOAc/hexanes |
| 35 | (CH₃)₂CHCH₂— | 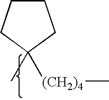 | 9 | R_{f} = 0.35, 2:3 EtOAc/hexanes |
| 36 | (CH₃)₂CHCH₂— | —CH(n-C₄H₉)(CH₂)₄— | 9 | R_{f} = 0.40, 2:3 EtOAc/hexanes |
| 37 | (CH₃)₂CHCH₂— | —CH(CH(CH₃)CH₂CH₂CH₃)(CH₂)₄— | 15 | R_{f} = 0.18, 2:3 EtOAc/hexanes |
| 38 | c-pentyl | 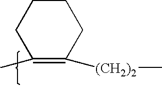 | 15 | R_{f} = 0.24, 2:3 EtOAc/hexanes |
| 39 | (CH₃)₂CHCH₂— | —CH(CH₃)CH₂C(CH₃)₂— | 15 | R_{f} = 0.18, 2:3 EtOAc/hexanes |
| 40 | (CH₃)₂CHCH₂— | 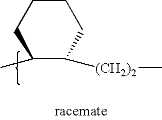 | 14 | R_{f} = 0.18, 2:3 EtOAc/hexanes |
| 41 | (CH₃)₂CHCH₂— | 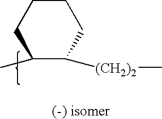 | 14 | R_{f} = 0.18, 2:3 EtOAc/hexanes |
| 42 | (CH₃)₂CHCH₂— | 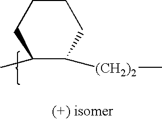 | 14 | R_{f} = 0.18, 2:3 EtOAc/hexanes |
| 43 | (CH₃)₂CH— | 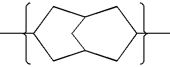 | 15 | R_{f} = 0.28, 2:3 EtOAc/hexanes |
| 44 | (CH₃CH₂)₂CH— | 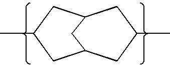 | 9 | R_{f} = 0.42, 2:3 EtOAc/hexanes |
| 45 | (CH₃)₂CHCH₂— | 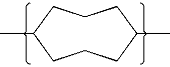 | 8 (KOtBu in place of NaH) | GC/MS m/z,: 209 [M⁺] |

### EXAMPLE 46

Preparation of 2,2-Dimethyl-N-(2-methyl-4-nitrophenyl)propanamide

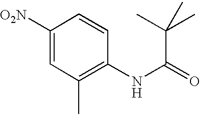

To a solution of (15.0 g, 98.6 mmol) 2-methyl-4-nitroaniline and (15.1 mL, 108.5 mmol) triethylamine in 150 mL CH₂Cl₂, trimethylacetyl chloride (12.1 mL, 98.6 mmol) was added dropwise over 30 min at 0° C. under argon. The reaction mixture was stirred at room temperature for 2 h whereupon the reaction turned from a yellow slurry to nearly clear yellow solution. The reaction mixture was allowed to reflux for 16 h, then additional triethylamine (2.7 mL, 19.7 mmol) and trimethylacetyl chloride (3.04 mL, 24.7 mmol) were added dropwise and refluxed for another 3 h. The reaction mixture was cooled to room temperature, washed twice with 2N HCl, twice with water, twice with saturated sodium bicarbonate, and twice with saturated NaCl. The organic layer was dried (Na₂SO₄) and concentrated in vacuo to give 23.5 g crude product, purified by trituration with 5% EtOAc in hexanes to give 20.1 g (86%) of white solid. R_{f}=0.4 (20% EtOAc/hexanes); MS m/z, 236 [M⁺]; ¹H NMR (CDCl₃, δ=7.24): 8.35 (d, J=8.8 Hz, 1H), 8.06 (m, 2H), 7.49 (br s, 1H), 2.34 (s, 1H), and 1.39 (s, 9H).

### EXAMPLE 47

### Preparation of 2-Methyl-N-(2-methyl-4-nitrophenyl)propanamide

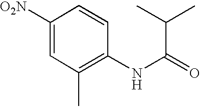

To a cold solution of 2-methyl-4-nitroaniline (440 g, 0.263 mol) and triethylamine (47.7 mL, 0.342 mol) in 500 mL CH₂Cl₂, isobutyryl chloride (34.4 mL, 0.328 mol), was added dropwise under argon over 1 h at 0° C. The reaction mixture was then stirred at room temperature for 72 h, becoming a dark brown solution. The reaction mixture was quenched with 2N HCl and diluted with CH₂Cl₂. The organic was washed successively with two portions of 2N HCl, two portions of water, two portions of saturated sodium bicarbonate, and two portions of saturated NaCl. The organic extract was dried by (Na₂SO₄) and concentrated in vacuo to give 56 g crude compound. The crude yellow compound was recrystallized twice with isopropanol to give two crops of crystalline product, 46.8 g and 5.7 g (total 52.5 g, 90%). MS m/z, 222 [M⁺]; ¹H NMR (CDCl₃, δ=7.24): 8.35 (d, J=9.5 Hz, 1H), 8.08 (m, 2H), 7.20 (br s, 1H), 2.60 (m, 1H), 2.35 (s, 3H), 1.30 (s, 3H), and 1.27 (s, 3H); ¹³C NMR (CDCl₃, δ=77.0):175.3, 143.4, 142.0, 127.5, 125.5, 121.4, 36.8, and 17.6.

Utilizing the procedures described for Example 47 above and employing the appropriate startng materials, the examples 48-52 shown in Table 2 below were similarly prepared.

TABLE 2
Preparative Examples of Anilides

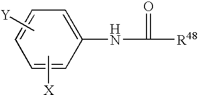

| | | | | | Method of |
|---|---|---|---|---|---|
| Ex. No. | R⁴⁸ | X | Y | Characterization | Example |
| 47 | i-Pr | 2-Me | 4-NO₂ | MS m/z, 222 [M⁺] | 47 |
| 48 | c-Pent | 2-Me | 4-NO₂ | MS m/z, 248 [M⁺] | 47 |
| 49 | c-Hex | 2-Me | 4-NO₂ | — | 47 |
| 50 | c-Bu | 2-Me | 4-NO₂ | — | 47 |
| 51 | c-Pr | 2-Me | 4-NO₂ | — | 47 |
| 52 | Ph | 2-Me | 4-NO₂ | — | 47 |

### EXAMPLE 53

### Preparation of N-(4-(2-Ethylbutyl)-4-azatricyclo[4.3.1.1^{3,8})uodec-5-ylidene]-2-methyl-4-nitroaniline

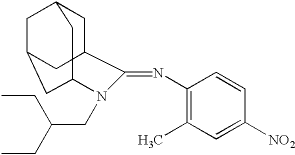

A solution of 0.25 g (1.00 mmol) of 4-(2-ethyl)butyl-4-azatricyclo-[4.3.1.1^{3,8}]undecan-5-one in 20 mL of CH₂Cl₂ was chilled to −78° C. To this was added 0.200 g (1.30 mmol) of phosphorus oxychloride and the reaction was brought to room temperature and stirred for 1 h. To this was then added 0.458 g (3.00 mmol) of 2-methyl-4-nitroaniline and the mixture was allowed to stir for 3 days at room temperature. The reaction mixture was then quenched with 20 ML of saturated potassium carbonate, and the organic phase was separated and dried (Na₂SO₄). The solution was filtered and concentrated in vacuo. The resulting residue was purified by chromatography eluting with 10% EtOAc/hexanes, affording a thick oil (0.107 g, 28%). R_{f}=0.56 (10% EtOAc/hexanes); MS (FAB) m/z, 384 [M+H]⁺.

### EXAMPLE 54

### Preparation of N-(4-Isobutyl-4-azatricyclo[4.3.1.1^{3,8}]undec-5-ylidene)-2-methyl-4-nitroaniline

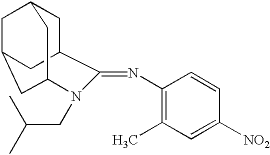

To a solution of 24.9 g (0.113 moles) 4-(2-methyl)propyl-4-azatricyclo-[4.3.1.1^{3,8}]undecan-5-one in 500 mL of CH₂Cl₂ was added 22.4 g (3.63 mL,0.146 moles) phosphorus oxychloride. The mixture was then allowed to heat at 70° C. for 1 h. To this was then added 18.6 g (0.122 moles) of 2-methyl-4-nitroaniline, followed quickly by 22.8 g (31.4 mL, 0.225 moles) triethylamine, and the mixture heated at 70° C. for 5 h. The reaction mixture was cooled, filtered and the CH₂Cl₂ filtrate was concentrated in vacuo leaving a dark residue. This residue was dissolved in 250 mL of CH₂Cl₂ and washed with 2N NaOH. The organic phase was separated, dried (Na₂SO₄) and concentrated in vacuo leaving a residue that was purified by chromatography, eluting with 10% EtOAc/hexanes to afford the product (24.0 g, 60%). R_{f}=0.7110% EtOAc/hexanes; mp 101-102° C.; MS (FAB) m/z, 356 [M+H]⁺.

### EXAMPLE 55

### Preparation of N-(4-Cyclopentyl-4-azatricyclo[4.3.1.1^{3,8}]undec-5-ylidene)-2-methyl-4-nitroaniline

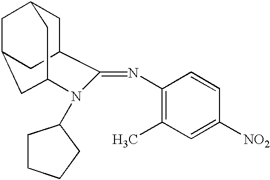

To a solution of 0.757 g (3.25 mmol) of 4-cyclopentyl-4-azatricyclo[4.3.1.1^{3,8}]undecan-5-one in 20 mL CH₂Cl₂ was added 0.65 g (0.40 mL,4.24 mmol) phosphorus oxychlolide. This mixture was allowed to stir for 1 h at room temperature. To this was added 0.41 g 2-methyl-4-nitroaniline and stirring was continued for 16 h at room temperature. The reaction mixture was then washed with 2N NaOH, the organic layer separated, dried (Na₂SO₄), filtered and concentrated in vacuo. The residue was purified on a silica gel column eluting with 10% EtOAc/hexanes to afford the product (0.060 g, 5%). mp 149-152° C.; R_{f}=0.50, 10% EtOAc/hexanes.

### EXAMPLE 56

### Preparation of N-[2,2-Dimethyl-1-(4-morpholinyl)propylidene]-2-methyl-4-nitroaniline

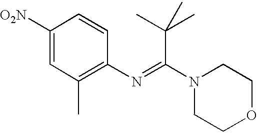

To a solution of 2,2-dimethyl-N-(2-methyl-4-nitrophenyl)propanamide (1.0 g, 4.2 mmol) in thionyl chloride (5 mL, 68.5 mmol) was added one drop of DMP. The reaction mixture was stirred for 16 h then the excess thionyl chloride was removed in vacuo. The crude reaction was diluted with 2 mL CH₂Cl₂, then morpholine (2 mL, 22.9 mmol) was added dropwise via syringe over a 5-minute period. The reaction mixture was stirred at room temperature for 16 h and a solid formed. The reaction was diluted with CH₂Cl₂, washed successively with 1 N NaOH, water, and saturated NaCl solution. The organic layer was dried (Na₂SO₄) and concentrated in vacuo to give 1.6 g crude product, which was chromatographed, eluting with 50% Et₂O/hexane elution to obtain 0.9 g (70%) product. R_{f}=0.43 (50% EtOAc/hexane); MS m/z, 305 [M⁺]; ¹H NMR (CDCl₃, δ=7.24): 7.99 (d, J=2.5 Hz, 1H), 7.95 (dd, J=2.7, 8.6 Hz, 1H), 6.48 (d, J=8.5 Hz, 1H), 3.58 (t, J=4.8 Hz, 4H), 3.01 (t, J=4.6 Hz, 4H), 2.14 (s, 3H) and 1.33 (s, 9H).

### EXAMPLE 57

### Preparation of N,N-Diisobutyl-2,2-dimethyl-N′-(2-methyl-4-nitrophenyl)propanimidamide

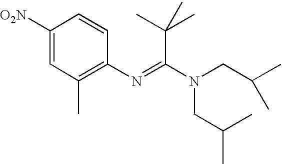

2,2-Dimethyl-N-(2-methyl-4-nitrophenyl)propanamide (1.0 g, 4.2 mmol) was dissolved in 3.75 mL (51.4 mmol) thionyl chloride, and the reaction mixture was stirred for 16 h. The excess thionyl chloride was removed by rotary evaporation and the crude residue was diluted with 5 mL CH₂Cl₂. Diisobutylamine (2 mL, 11.5 mmol) was added slowly with a syringe and the reaction mixture was stirred at room temperature for 72 h during which time a solid formed. The reaction mixture was diluted with CH₂Cl₂ and washed successively with 2N HCl, saturated NaHCO₃, and saturated NaCl. The organic layer was dried (Na₂SO₄) and concentrated in vacuo to give 2.02 g crude product. Chromatography with 10% EtOAc/hexane elution gave 1.0 g (68%) of the final product. R_{f}=0.64 (20% EtOAc/hexane); MS m/z, 347 [M⁺]; ¹H NMR (CDCl₃, δ=7.24): 8.00 (d, J=2.4 Hz, 11), 7.93 (dd, J=2.4, 8.6 Hz, 1H), 6.48 (d, J=8.6 Hz, 1H), 2.82 (br s, 4H), 2.15 (s, 3H), 1.79 (m, 2H), 1.35 (s, 9H), and 0.81 (d, J=6.6 Hz, 12H).

### EXAMPLE 58

### Preparation of N-isobutyl-N,2-dimethyl-N′-(2-methyl-4-nitrophenyl)propanimidamide

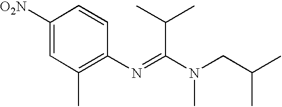

To a solution of N-isobutyl-2-methyl-N′-(2-methyl-4-nitrophenyl)propanimidamide (400 mg 1.76 rnmol) in 2 mL DMF, sodium hydride (66.7 mg, 2.64 mmol) was added. The reaction mixture turned orange and gas formed. The reaction was stirred at room temperature for 1.5 h and methyl iodide (270 μl, 4.40 mmol) was added via synnge and the reaction mixture was stirred at room temperature for 45 minutes. The reaction mixture was then diluted with CH₂Cl₂ and slowly quenched with water. The organic layer was separated, washed with twice water and twice with saturated NaCl solution, then dried (Na₂SO₄). The solution was filtered and concentrated in vacuo to give 360 mg (86%) of a yellow oil after chromatography with 15% EtOAc/hexane elution. R_{f}=0.73 (50% EtOAc/hexane); MS m/z, 291 [M⁺], 276 [M−CH₃]⁺; ¹H NMR (CDCl₃, δ=7.24): 7.99 (d, J=2.2 Hz, 1H), 7.93 (dd, J=2.2, 8.6 Hz, 1H), 6.54 (d, J=8.6 Hz, 1H), 3.20 (d, J=7.4 Hz, 2H), 2.86 (m, 1H), 2.84 (s, 3H), 2.12 (s, 3H), 2.03 (m, 1H), 1.13 (d, J=7.3 Hz, 6H), and 0.91 (d, J=6.6 Hz, 6H).

### EXAMPLE 59

### Preparation of N,N-Diisobutyl-N′-(2-methyl-4-nitrophenyl)-2-propynimidamide

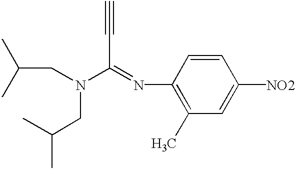

To a solution of ethynyl magnesium bromide (3.9 mL, 1.96 mmol) in THF (2 mL) was added a suspension of N,N-diisobutyl-N′-(2-methyl-4nitrophenyl)urea (300 mg, 0.98 mmol) in THF (2 mL) dropwise at room temp, under argon. The mixture was stirred at room temp for 18 h, slowly quenched with water and then extracted with Et2O. The organic layer was washed with saturated sodium bicarbonate solution, dried (MgSO₄), filtered and concentrated in vacuo. The residue was purified by chromatography, eluting with 5:2 hexane-EtOAc. The fractions containing product were concentrated in vacuo giving an orange oil (98 mg, 32%). MS (EI) m/z, 314 [M+H]⁺.

### EXAMPLE 60

### Preparation of 2-Methyl-N-[1-(2-methyl-1-propenyl)-2-azepanylidene]-4-nitroaniline

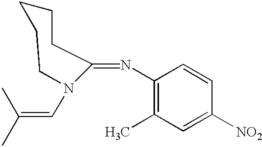

To 0.10 g, 0.283 mmol of 2-methyl-N-[1-(2-methyl-2-propenyl)-2-azepanylidene]-4-nitroaniline was added 1 mL of polyphosphoric acid. This was allowed to heat at 90° C. for 5 h. The reaction mixture was then allowed to come to room temperature and stir for 16 h longer, then diluted with 10 mL of water and neutralized with 2N NaOH. The mixture was extracted with 40 mL of CH₂Cl₂, separated, dried and concentrated in vacuo to a yellow oil (0.098 g, 98%). TLC (10% EtOAc/hexanes), R_{f}=0.09.

### EXAMPLE 61

### Preparation of N,N-Diisobutyl-N′-(2-methyl-4-nitrophenyl)ethanimidamide Hydrochloride

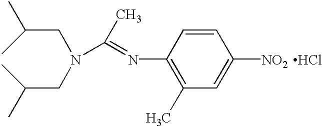

N,N-Diisobutyl-N′-(2-methyl-4-nitrophenyl)ethanimidamide (230 mg), prepared as in example 57 from N-(2-methyl-4-nitorphenylacetamide and diisobutylamine, was dissolved in CH₂Cl₂ and 1M HCl in Et₂O (about 5 mL) was added with a pipette. The resulting colorless solution was concentrated to give 257 mg (100%) product as a foam. HPLC (Method A: 10 cm Dynamax C18; 1.5 mL/min; 254 miu; water (0.5% TFA) to acetonitrile (0.5% TFA) over 10 min, hold 5 min), Rt 7.41 min, 99.0%.

### EXAMPLE 62

### Preparation of N-(1-Isobutyl-1-azaspiro[4.4]non-2-ylidene)-2-methyl-4-nitroaniline

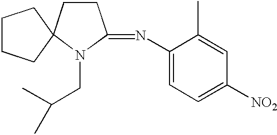

To N-(1-azaspiro[4.4]non-2-ylidene)-2-methyl-4-nitroaniline (19.4 mg, 0.071 mmol) was added cesium carbonate (69 mg, 0.213 mmol), isobutyl bromide (1 mL, 9.2 mmol) and N,N-dimethylformamide (2 mL) and the mixture was heated to 60° C. for 2 h. The reaction was cooled to ambient temperature and partitioned between EtOAc and 1% sodium bicarbonate solution The separated organic layer was washed with saturated NaCl, dried (MgSO4) and filtered. The product was obtained after chromatography on silica gel, eluting with hexane-EtOAc mixtures giving 14.7 mg, 0.045 mmol(63%). HPLC (Method A: 10 cm Dynamax C18; 1.5 ml/min; 254 nm; water(0.5%TFA) to acetonitrile (0.5% TFA) over 10 min, hold 5 min), Rt=7.87 min, 99.3%; MS (CD) mlz, 330 [M+H]⁺.

Utilizing the above procedures of examples 53-62 and substituting the appropriate starting materials, examples 63-164 shown below in Table 3 were prepared.

TABLE 3
Preparation of Amidines

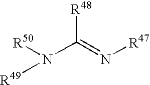

| Ex. | | | | | | Method of |
|---|---|---|---|---|---|---|
| No. | R⁴⁹ | R⁵⁰ | R⁴⁸ | R⁴⁷ | Characterization^{a,b} | Example |
| 53 | (Et)₂CHCH₂— | 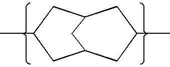 | | 2-Me-4-NO₂-phenyl | R_{f} = 0.56, 10% EtOAc/hexanes MS (FAB) m/z, 384 [M + H]⁺ | 53 |
| 54 | i-Bu | 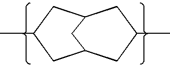 | | 2-Me-4-NO₂- phenyl | R_{f} = 0.71, 10% EtOAc/hexanes MS (FAB) m/z, 356 [M + H]⁺ | 54 |
| 55 | c-Pent | 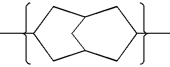 | | 2-Me-4-NO₂- phenyl | R_{f} = 0.50, EtOAc/hexanes mp 149-152° C. | 55 |
| 56 | —(CH₂)₂O(CH₂)₂— | | t-Bu | 2-Me-4-NO₂- | R_{f} = 0.43, 50% | 56 |
| | | | | phenyl | EtOAc/hexanes | |
| | | | | | MS m/z, 305 [M⁺] | |
| 57 | (CH₃)₂CHCH₂— | i-Bu- | t-Bu | 2-Me-4-NO₂- | R_{f} = 0.64, 20% | 57 |
| | | | | phenyl | EtOAc/hexanes | |
| | | | | | MS m/z, 347 [M⁺] | |
| 58 | Me | i-Bu | i-Pr | 2-Me-4-NO₂— | R_{f} = 0.73, 50% | 58 |
| | | | | phenyl | EtOAc/hexanes | |
| | | | | | MS m/z, 291 [M⁺] | |
| 59 | i-Bu | i-Bu | HC≡C— | 2-Me-4-NO₂— | MS (EI) m/z, 314 [M + H]⁺ | 59 |
| | | | | phenyl | | |
| 60 | (Me)₂C═CH— | —(CH₂)₅— | | 2-Me-4-NO₂- | R_{f} = 0.09, 10% | 60 |
| | | | | phenyl | EtOAc/hexanes | |
| 61 | i-Bu | i-Bu | Me | 2-Me-4-NO₂- | as HCl salt | 61 |
| | | | | phenyl | Rₜ = 7.41 | |
| 62 | i-Bu | 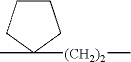 | | 2-Me-4-NO₂- phenyl | Rt = 7.87, Method A MS (CI) m/z, 330 [M + H]⁺ | 62 |
| 63 | —(CH₂)₄— | | i-Pr | 2-Me-4-NO₂- | R_{f} = 0.50, 50% | |
| | | | | phenyl | EtOAc/hexanes | |
| | | | | | MS m/z, 275 [M⁺] | |
| 64 | —CH₂CH(CH₃)CH₂CH(CH₃)CH₂— | | i-Pr | 2-Me-4-NO₂- | R_{f} = 0.73, 50% | 56 |
| | 2:1 cis:trans | | | phenyl | EtOAc/hexanes | |
| | | | | | MS m/z, 318 [M + H]⁺ | |
| 65 | —CH₂CH(CH₃)CH₂CH(CH₃CH₂— | | i-Pr | 2-Me-4-NO₂- | R_{f} = 0.73, 50% | 56 |
| | cis | | | phenyl | EtOAc/hexanes | |
| | | | | | MS m/z, 318 [M + H]⁺ | |
| 66 | —CH₂CH(CH₃)CH₂CH(CH₃)CH₂— | | i-Pr | 2-Me-4-NO₂- | R_{f} = 0.73, 50% | 56 |
| | trans | | | phenyl | EtOAc/hexanes | |
| | | | | | MS m/z, 318 [M + H]⁺ | |
| 67 | —(CH₂CH(CH₃)OCH(CH₃CH₂— | | i-Pr | 2-Me-4-NO₂— | R_{f} = 0.73, 50% | 56 |
| | cis | | | phenyl | EtOAc/hexanes | |
| | | | | | MS m/z, 293 [M⁺] | |
| 68 | —CH₂CH₂SCH_{2—} | | i-Pr | 2-Me-4-NO₂— | R_{f} = 0.46, 50% | 56 |
| | | | | phenyl | EtOAc/hexanes | |
| | | | | | MS m/z, 293 [M⁺] | |
| 69 | —CH(CH₃)CH₂CH₂CH₂CH(CH₃)— | | i-Pr | 2-Me-4-NO₂— | R_{f} = 0.68, | 56 |
| | | | | phenyl | 50% EtOAc/hexanes | |
| | | | | | MS m/z, 317 [M⁺] | |
| 70 | —CH₂CH₂CH(Ph)CH₂CH₂— | | i-Pr | 2-Me-4-NO₂— | R_{f} = 0.55, | 56 |
| | | | | phenyl | 50% EtOAc/hexanes | |
| | | | | | MS m/z, 366 [M + H]⁺ | |
| 71 | —(CH₂)₆— | | i-Pr | 2-Me-4-NO₂— | R_{f} = 0.57, 50% | 56 |
| | | | | phenyl | EtOAc/hexanes | |
| | | | | | MS m/z, 303 [M⁺] | |
| 72 | —(CH₂)₃C(CH₃)₂CH₂— | | i-Pr | 2-Me-4-NO₂- | R_{f} = 0.78, 50% | 56 |
| | | | | phenyl | EtOAc/hexanes | |
| | | | | | MS m/z, 317 [M⁺] | |
| 73 | —(CH₂)₇— | | i-Pr | 2-Me-4-NO₂- | R_{f} = 0.57, 50% | 56 |
| | | | | phenyl | EtOAc/hexanes | |
| | | | | | MS m/z, 303 [M⁺] | |
| 74 | 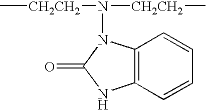 | | i-Pr | 2-Me-4-NO₂- phenyl | R_{f} = 0.24, 5% MeOH/CH2Cl2 MS m/z, 422 [M⁺] | A |
| 75 | H | i-Bu | i-Pr | 2-Me-4-NO₂— | R_{f} = 0.39, 50% | 56 |
| | | | | phenyl | EtOAc/hexanes | |
| | | | | | MS m/z, 277 [M⁺] | |
| 76 | Et | i-Bu | i-Pr | 2-Me-4-NO₂- | R_{f} = 0.52, 25% | 58 |
| | | | | phenyl | EtOAc/hexanes | |
| | | | | | MS m/z, 306 [M⁺] | |
| 77 | n-Pr | i-Bu | i-Pr | 2-Me-4-NO₂- | R_{f} = 0.78, 25% | 58 |
| | | | | phenyl | EtOAc/hexanes | |
| | | | | | MS m/z, 319 [M⁺] | |
| 78 | i-Bu | 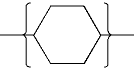 | | 2-Me-4-NO₂- phenyl | R_{f} = 0.40, 10% EtOAc/hexanes MS (EI) m/z, 309 [M⁺] | 53 |
| 79 | i-Bu | 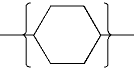 | | 2-Me-4-NO₂- phenyl | R_{f} = 0.35, 10% EtOAc/hexanes MS (FAB) m/z, 316 [M + H]⁺ | 53 |
| 80 | i-Bu | 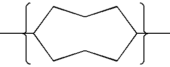 | | 2-Me-4-NO₂- phenyl | R_{f} = 0.40, 20% EtOAc/hexanes MS (EI) m/z, 344 [M + H]⁺ | 53 |
| 81 | i-Bu | i-Bu | Et | 2-Me-4-NO₂- | as HCl salt | 53, 61 |
| | | | | phenyl | Rₜ = 7.57 min, Method A | |
| 82 | i-Bu | i-Bu | n-Bu | 2-Me-4-NO₂- | as HCl salt | 53, 61 |
| | | | | phenyl | Rₜ = 8.01 min, Method A | |
| 83 | i-Bu | i-Bu | n-Pr | 2-Me-4-NO₂- | as HCl salt | 53, 61 |
| | | | | phenyl | Rₜ = 7.66 min, Method A | |
| 84 | i-Bu | i-Bu | i-Pr | 2-Me-4-NO₂- | Rₜ = 7.83 min, Method A | 53 |
| | | | | phenyl | | |
| 85 | i-Bu | i-Bu | i-Pr | 2-Me-4-NO₂- | as HCL salt | 53, 61 |
| | | | | phenyl | Rₜ = 7.83 min, Method A | |
| 86 | i-Bu | i-Bu | i-Bu | 2-Me-4-NO₂- | as HCL salt | 53, 61 |
| | | | | phenyl | Rₜ = 7.96 min, Method A | |
| 87 | i-Pr | i-Pr | i-Pr | 2-Me-4-NO₂- | as HCL salt | 53, 61 |
| | | | | phenyl | Rₜ = 7.00 min, Method A | |
| 88 | n-Bu | n-Bu | i-Pr | 2-Me-4-NO₂- | as HCL salt | 53, 61 |
| | | | | phenyl | Rₜ = 7.73 min | |
| 89 | s-Bu | s-Bu | i-Pr | 2-Me-4-NO₂- | as HCL salt | 53, 61 |
| | | | | phenyl | Rₜ = 7.83 min, Method A | |
| 90 | H | 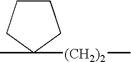 | | 2-Me-4-NO₂- phenyl | R_{f} = 0.25, 5% MeOH/CH₂Cl₂ | 53 |
| 91 | H | 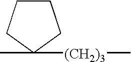 | | 2-Me-4-NO₂- phenyl | — | 53 |
| 92 | i-Bu | 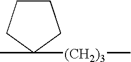 | | 2-Me-4-NO₂- phenyl | Rₜ = 7.93 min, Method A | 62 |
| 93 | i-Bu | 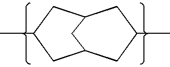 | | 2-Me-4-NO₂- phenyl | as HCL salt of Example 47 | 61 |
| 94 | H₂C═C(CH₃)CH₂— | —(CH₂)₅— | | 2-Me-4-NO₂- | R_{f} = 0.22, 10% | 46 |
| | | | | phenyl | EtOAc/hexanes | |
| | | | | | MS (FAB) m/z, 302 [M + H]⁺ | |
| | | | | | mp 70-72° C. | |
| 95 | H₂C═C(CH₃)CH₂— | 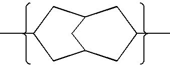 | | 2-Me-4-NO₂- phenyl | R_{f} = 0.10, 10% EtOAc/hexanes MS (FAB) m/z, 354 [M + H]⁺ | 46 |
| 96 | (Me)₂C═CH— | 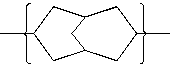 | | 2-Me-4-NO₂- phenyl | R_{f} = 0.09, 10% EtOAc/hexanes MS (FAB) m/z, 354 [M + H]⁺ | |
| 97 | i-Bu | 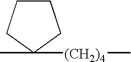 | | 2-Me-4-NO₂- phenyl | R_{f} = 0.62, 10% EtOAc/hexanes MS (FAB) m/z, 358 [M + H]⁺ | 53 |
| 98 | s-Bu | —(CH₂)₃— | | 2-Me-4-NO₂- | R_{f} = 0.15, 10% | 53 |
| | | | | phenyl | EtOAc/hexanes | |
| | | | | | MS m/z, 276 [M + H]⁺ | |
| 99 | 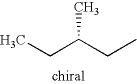 | 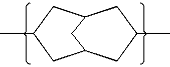 | | 2-Me-4-NO₂- phenyl | R_{f} = 0.36, 10% EtOAc/hexanes MS (FAB) m/z, 370 [M + H]⁺ | 53 |
| 100 | Me | 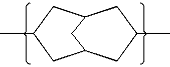 | | 2-Me-4-NO₂- phenyl | R_{f} = 0.11, 10% EtOAc/hexanes mp 127-128° C. | 53 |
| 101 | i-Bu | —(CH₂)₄— | | 2-Me-4-NO₂- | R_{f} = 0.25, 10% | 53 |
| | | | | phenyl | EtOAc/hexanes | |
| | | | | | mp 149-152° C. | |
| 102 | H₂C═C(CH₃)CH₂— | —(CH₂)₃— | | 2-Me-4-NO₂- | R_{f} = 0.13, 10% | 53 |
| | | | | phenyl | EtOAc/hexanes | |
| | | | | | MS (FAB) m/z, 274 [M + H]⁺ | |
| 103 | H₂C═C(CH₃)CH₂— | —(CH₂)₅— | | 2-Me-4-NO₂- | R_{f} = 0.22, 10% | 53 |
| | | | | phenyl | EtOAc/hexanes | |
| | | | | | MS (FAB) m/z, 302 [M + H]⁺ | |
| | | | | | mp 70-72° C. | |
| 104 | H₂C═C(CH₃)CH₂— | —(CH₂)₄— | | 2-Me-4-NO₂- | R_{f} = 0.18, 10% | 53 |
| | | | | phenyl | EtOAc/hexanes | |
| | | | | | MS (EI) m/z, 287 [M⁺] | |
| 105 | H₂C═C(CH₃)CH₂— | —(CH₂)₆— | | 2-Me-4-NO₂- | R_{f} = 0.28, 10% | 53 |
| | | | | phenyl | EtOAc/hexanes | |
| | | | | | MS (FAB) m/z, 318 [M + H]⁺ | |
| | | | | | mp 77-79° C. | |
| 106 | H₂C═C(CH₃)CH₂— | —(CH₂)₇— | | 2-Me-4-NO₂- | R_{f} = 0.93, 10% | 53 |
| | | | | phenyl | EtOAc/hexanes | |
| | | | | | MS m/z, 330 [M + H]⁺ | |
| 107 | (Me)₂C═CH₂— | —(CH₂)₅— | | 2-Me-4-NO₂- | R_{f} = 0.09, 10% | 53 |
| | | | | phenyl | EtOAc/hexanes | |
| | | | | | MS m/z, 302 [M + H]⁺ | |
| 108 | H₂C═C(CH₃)CH₂— | 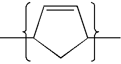 | | 2-Me-4-NO₂- phenyl | R_{f} = 0.08, 10% EtOAc/hexanes MS (EI) m/z, 297 [M + H]⁺ | 53 |
| 109 | H₂C═C(CH₃)CH₂— | 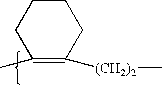 | | 2-Me-4-NO₂- phenyl | R_{f} = 0.47, 10% EtOAc/hexanes MS (FAB) m/z, 340 [M + H]⁺ | 53 |
| 110 | (Et)₂CHCH₂— | 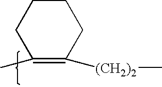 | | 2-Me-4-NO₂- phenyl | R_{f} = 0.51, 10% EtOAc/hexanes MS (FAB) m/z, 370 [M + H]⁺ | 53 |
| 111 | (Et)₂CHCH₂— | 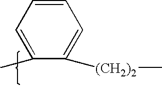 | | 2-Me-4-NO₂- phenyl | R_{f} = 0.45, 10% EtOAc/hexanes MS (FAB) m/z, 366 [M + H]⁺ | 53 |
| 112 | (Et)₂CHCH₂— | 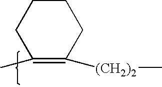 | | 2-Me-4-NO₂- phenyl | R_{f} = 0.41, 10% EtOAc/hexanes MS (FAB) m/z, 372 [M + H]⁺ | 53 |
| 113 | i-Bu | 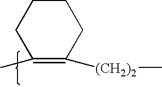 | | 2-Me-4-NO₂- phenyl | R_{f} = 0.66, 10% EtOAc/hexanes MS (EI) m/z, 341 [M⁺] | 53 |
| 114 | i-Bu | —CH(n-Bu)(CH₂)₄— | | 2-Me-4-NO₂- | R_{f} = 0.72, 10% | 53 |
| | | | | phenyl | EtOAc/hexanes | |
| | | | | | MS (CI) m/z 360 [M + H]⁺ | |
| 115 | i-Bu | —CH(CH(CH₃)CH₂CH₃)(CH₂)₄— | | 2-Me-4 | R_{f} = 0.73, 10% | 53 |
| | | | | NO₂-phenyl | EtOAc/hexanes | |
| | | | | | MS (EL) m/z, 359 [M⁺] | |
| 116 | c-Pent | 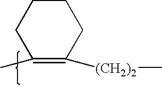 | | 2-Me-4-NO₂- phenyl | R_{f} = 0.45, 10% EtOAc/hexanes MS (EI) m/z, 353 [M⁺] | 53 |
| 117 | i-Bu | —CH(Me)CH₂C(Me)₂— | | 2-Me-4-NO₂- | R_{f} = 0.60, | 53 |
| | | | | phenyl | 10% EtOAc/hexanes | |
| | | | | | MS (FAB) m/z, 318 [M +H]⁺ | |
| 118 | i-Bu | 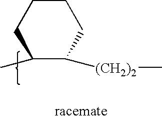 | | 2-Me-4-NO₂- phenyl | R_{f} = 0.26, 10% EtOAc/hexanes MS (EI) m/z, 343 [M⁺] | 53 |
| 119 | i-Bu | 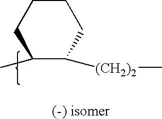 | | 2-Me-4-NO₂- phenyl | R_{f} = 0.26, 10% EtOAc/hexanes MS (CI) m/z, 344 [M + H]⁺ | 53 |
| 120 | i-Bu | 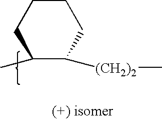 | | 2-Me-4-NO₂- phenyl | R_{f} = 0.26, 10% EtOAc/hexanes MS (CI) m/z, 344 [M + H]⁺ | 53 |
| 121 | i-Pr | 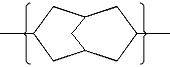 | | 2-CF₃-4-NO₂- phenyl | Rₜ = 3.509, Method B | 53 |
| 122 | i-Pr | 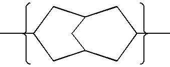 | | 2-Me-4-NO₂- phenyl | Rₜ = 2.97, Method B | 53 |
| 123 | i-Bu | 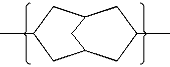 | | 2-Cl-4-NO₂- phenyl | Rₜ = 3.32, Method B | 53 |
| 124 | i-Pr | 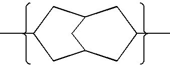 | | 4-NO₂- phenyl | Rₜ = 2.87, Method B | 53 |
| 125 | i-Pr | 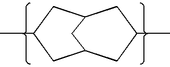 | | 2-Cl-4-NO₂- phenyl | Rₜ = 2.90, Method B | 53 |
| 126 | i-Bu | 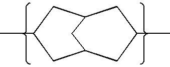 | | 2-Me-4-F- phenyl | Rₜ = 3.25, Method B | 53 |
| 127 | i-Bu | 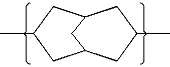 | | 2-Et-4-CN- phenyl | Rₜ = 3.17, Method B | 53 |
| 128 | (Et)₂CHCH₂— | 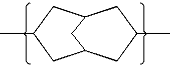 | | 2-Cl-4-NO₂- phenyl | Rₜ = 3.61, Method B | 53 |
| 129 | i-Pr | 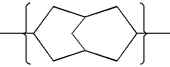 | | 2-Me-4-CN- phenyl | Rₜ = 2.91, Method B | 53 |
| 130 | H₂C═C(CH₃)CH₂— | 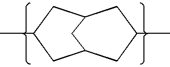 | | 2-Cl-4-NO₂- phenyl | Rₜ = 3.11, Method B | 53 |
| 131 | (Et)₂CH— | 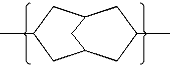 | | 4-NO₂-phenyl | Rₜ = 3.03, Method B | 53 |
| 132 | i-Pr | 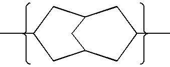 | | 2-Me-4-F- phenyl | Rₜ = 3.00, Method B | 53 |
| 133 | (Et)₂CH— | 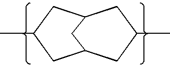 | | 2-Cl-4-NO₂- phenyl | Rₜ = 3.27, Method B | 53 |
| 134 | (Et)₂CHCH₂— | 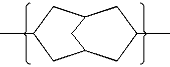 | | 2-Me-4-NO₂- phenyl | Rₜ = 3.62, Method B | 53 |
| 135 | i-Pr | 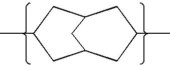 | | 3-CF₃-4-NO₂- phenyl | Rₜ = 3.09, Method B | 53 |
| 136 | i-Bu | 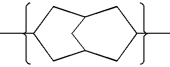 | | 3-CF₃-4-NO₂- phenyl | Rₜ = 3.24, Method B | 53 |
| 137 | H₂C═C(CH₃)CH₂— | 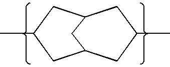 | | 3-CF₃-4-NO₂- phenyl | Rₜ = 3.09, Method B | 53 |
| 138 | (Et)₂CH₂— | 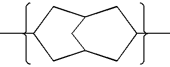 | | 2-Et-4-CN- phenyl | Rₜ = 3.21, Method B | 53 |
| 139 | H₂C═C(CH₃)CH₂— | 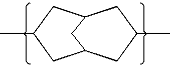 | | 4-NO₂-phenyl | Rₜ = 2.87, Method B | 53 |
| 140 | (Et)₂CH— | 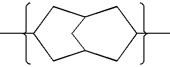 | | 2-Me-4-NO₂- phenyl | Rₜ = 3.22, Method B | 53 |
| 141 | H₂C═C(CH₃)CH₂— | 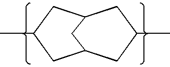 | | 2-Et-4-CN- phenyl | Rₜ = 3.07, Method B | 53 |
| 142 | (Et)₂CHCH₂— | 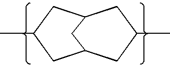 | | 2-quinolinyl | Rₜ = 3.84, Method B | 53 |
| 143 | i-Pr | 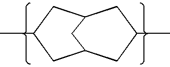 | | 3-NO₂-4-Cl- phenyl | Rₜ = 2.97, Method B | 53 |
| 144 | Me | t-Bu | i-Bu | 2-Me-4-NO₂- | R_{f} = 0.92, 1:4 | 56 |
| | | | | phenyl | EtOAc/hexanes | |
| | | | | | MS (EI) m/z, 294 [M⁺] | |
| 145 | Et | Et | Et | 2-Me-4-NO₂- | R_{f} = 0.66 1:2 | 53 |
| | | | | phenyl | EtOAc/hexanes | |
| | | | | | MS (FAB) m/z, 264 [M + H]⁺ | |
| 146 | c-Hex | c-Hex | i-Pr | 2-Me-4-NO₂- | R_{f} = 0.45, 1:2 | 56 |
| | | | | phenyl | EtOAc/hexanes | |
| | | | | | MS (FAB) m/z, 386 [M + H]⁺ | |
| 147 | i-Bu | i-Bu | c-Hex | 2-Me-4-NO₂- | R_{f} = 0.87, 1:4 | 56 |
| | | | | phenyl | EtOAc/hexanes | |
| | | | | | MS (EI) m/z, 373 [M⁺] | |
| 148 | i-Bu | i-Bu | c-Pent | 2-Me-4-NO₂- | R_{f} = 0.74, 1:4 | 56 |
| | | | | phenyl | EtOAc/hexanes | |
| | | | | | MS (FAB) m/z, 360 [M⁺] | |
| 149 | i-Bu | i-Bu | Ph | 2-Me-4-NO₂- | R_{f} = 0.66, 1:4 | 56 |
| | | | | phenyl | EtOAc/hexanes | |
| | | | | | MS (FAB) m/z, 368 [M + H]⁺ | |
| 150 | Me | Ph | i-Pr | 2-Me-4-NO₂- | R_{f} = 0.74, 1:2 | 56 |
| | | | | phenyl | EtOAc/hexanes | |
| | | | | | MS (FAB) m/z, 312 [M + H]⁺ | |
| 151 | t-Bu | PhCH₂— | i-Pr | 2-Me-4-NO₂- | R_{f} = 0.84, 1:4 | 56 |
| | | | | phenyl | EtOAc/hexanes | |
| | | | | | MS (EI) m/z, 367 [M⁺] | |
| 152 | Me | Me | Ph | 2-Me-4-NO₂- | R_{f} = 0.63, 1:2 | 56 |
| | | | | phenyl | EtOAc/hexanes | |
| | | | | | MS (EI) m/z, 283 [M⁺] | |
| 153 | Ph | Ph | Ph | 2-Me-4-NO₂- | R_{f} = 0.35, 1:4 | 56 |
| | | | | phenyl | EtOAc/hexanes | |
| | | | | | MS (FAB) m/z, 408 [M + H]⁺ | |
| 154 | i-Bu | i-Bu | c-Bu | 2-Me-4-NO₂- | R_{f} = 0.84, 1:2 | 56 |
| | | | | phenyl | EtOAc/hexanes | |
| | | | | | MS (FAB) m/z, 346 [M + H]⁺ | |
| 155 | i-Pr | c-Hex | i-Pr | 2-Me-4-NO₂- | R_{f} = 0.90, 1:4 | 56 |
| | | | | phenyl | EtOAc/hexanes | |
| | | | | | MS (EI) m/z, 345 [M⁺] | |
| 156 | Ph | Ph | i-Pr | 2-Me-4-NO₂- | R_{f} = 0.68, 1:4 | 56 |
| | | | | phenyl | EtOAc/hexanes | |
| | | | | | MS (EI) m/z, 373 [M⁺] | |
| 157 | PhCH₂— | PhCH₂— | i-Pr | 2-Me-4-NO₂- | R_{f} = 0.55, 1:4 | 56 |
| | | | | phenyl | EtOAc/hexanes | |
| | | | | | MS (EI) m/z, 401 [M⁺] | |
| 158 | i-Bu | c-Pent | i-Pr | 2-Me-4-NO₂- | R_{f} = 0.55, 1:4 | 56 |
| | | | | phenyl | EtOAc/hexanes | |
| | | | | | MS (EI) m/z, 345 [M⁺] | |
| 159 | i-Bu | i-Bu | PhC≡C— | 2-Me-4-NO₂- | — | 59 |
| | | | | phenyl | | |
| 160 | c-Pent | c-Pent | i-Pr | 2-Me-4-NO₂- | R_{f} = 0.73, 1:4 | 56 |
| | | | | phenyl | EtOAc/hexanes | |
| | | | | | LCMS m/z, 358 [M + H]⁺ | |
| 161 | i-Bu | i-Bu | c-Pr | 2-Me-4-NO₂- | R_{f} = 0.88, 1:4 | 56 |
| | | | | phenyl | EtOAc/hexanes | |
| | | | | | LCMS m/z, 332 [M + H]⁺ | |
| 162 | i-Bu | c-Pent | c-Pr | 2-Me-4-NO₂- | R_{f} = 0.80, 1:1 | 56 |
| | | | | phenyl | EtOAc/hexanes | |
| | | | | | LCMS m/z, 344 [M + H]⁺ | |
| 163 | c-Pent | c-Pent | c-Pr | 2-Me-4-NO₂- | R_{f} = 0.73, 1:4 | 56 |
| | | | | phenyl | EtOAc/hexanes | |
| | | | | | LCMS m/z, 356 [M + H]⁺ | |

| | | | | | |
|---|---|---|---|---|---|
| 164 | i-Bu | 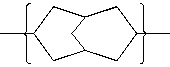 | 2-Me-4-F- phenyl | — | 56 |

^{a}R_{f} refers to the TLC retention factor characteristic of the product in the solvent system cited.
^{b}Rt refers to the retention time by HPLC using the follwing standard methods: Methods A: 10 cm Dynamax C₁₈ column, 1.5 ml/min; 254 nm; water(0.5% TFA) to acetonitrile (0.5% TFA) over 10 min, hold 5 min); Method B: 4.6 × 30 mm Brownlee C₁₈ column, water(0.1% TFA) to acetonitrile (0.1% TFA) over 6 minutes

Biological Protocol

The activity of a given compound to bind to the progesterone receptor can be assayed routinely according to the procedure disclosed below. This procedure was used to determine the progesterone binding activities of the compounds of the invention.

Progesterone Receptor Binding Assay

To siliconized glass test tubes cooled over an ice water bath was added binding buffer (100 mL; 50 mM Tris, pH 7.4, 10 mM molybdic acid, 2 mM EDTA, 150 mM NaCl, 5% Glycerol, 1% DMSO) containing various concentrations of a compound to be assayed, T47D cell cytosol (100 μL of a solution which will give at least 4000 cpm of binding) and ³H-progesterone (50 μL, 10 nM, NET-381). The mixture was incubated for 16 h at 4° C., and treated with charcoal (250 μL of a 0.5% mixture of 0.05% dextran-coated charcoal which had been washed twice with binding buffer). The resulting mixture was incubated for 10 min. at 4° C. The tubes were centrifuged (20 min at 2800×g) at 4° C. The supernatant was transferred into scintillation vials containing scintillation fluid (4 mL). Remaining ³H-progesterone was determined with a Packard 1900TR beta counter. Each assay included the following control groups: 1) total binding group (without compound), 2) non-specific binding group (with 400 nM progesterone), and 3) positive control group (with 2 nM progesterone or a known inhibitor).

The compounds of the present invention were found to cause greater than or equal to 30% inhibition of binding of ³H-progesterone to the progesterone receptor at a compound concentration of 200 nM. Activity ranges of the compounds of the present invention in the Progesterone Receptor Binding Assay at a compound concentration of 200 nM are listed in Table 4.

TABLE 4
Inhibitory Activity of Exemplified Compounds

| Compounds Which | Compounds Which | Compounds Which |
|---|---|---|
| Cause 30-59% | Cause 60-79% | Cause 80-100% |
| Inhibition at 200 nM | Inhibition at 200 nM | Inhibition at 200 mM |
| (Entry Number) | (Entry Number) | (Entry Number) |
| 56 | 58 | 53 |
| 64 | 59 | 54 |
| 68 | 60 | 55 |
| 70 | 66 | 57 |
| 71 | 67 | 61 |
| 75 | 72 | 62 |
| 78 | 73 | 65 |
| 79 | 86 | 69 |
| 97 | 96 | 74 |
| 98 | 107 | 76 |
| 100 | 113 | 77 |
| 101 | 117 | 80 |
| 102 | 121 | 81 |
| 104 | 126 | 82 |
| 108 | 144 | 83 |
| 114 | 147 | 84 |
| 115 | 157 | 85 |
| 116 | 160 | 87 |
| 120 | 164 | 88 |
| 127 | | 89 |
| 128 | | 92 |
| 129 | | 94 |
| 130 | | 95 |
| 131 | | 99 |
| 132 | | 103 |
| 133 | | 105 |
| 135 | | 106 |
| 136 | | 109 |
| 137 | | 110 |
| 138 | | 111 |
| 139 | | 112 |
| 140 | | |
| 141 | | 118 |
| 142 | | 119 |
| 143 | | 134 |
| 145 | | 148 |
| 146 | | 149 |
| 150 | | 153 |
| 151 | | 154 |
| 152 | | 158 |
| 156 | | 161 |
| | | 162 |
| | | 163 |

The invention may be embodied in other specific forms without departing from the spirit or essential characteristics thereof. The foregoing examples are included by way of illustration only. Accordingly, the scope of the invention is limited only by the scope of the appended claims.

What is claimed is:

1. A compound of the formula (I) 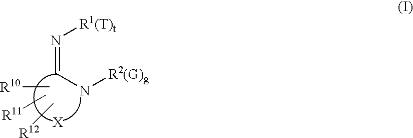
wherein
R¹
is selected from the group consisting of aryl of 6-12 carbon atoms and heteroaryl of 2-11 carbon atoms and 1-3 heteroatoms selected from the group consisting of N, S and O;
T
is selected from the group consisting of hydrogen, nitro, nitriue, alkyl of 1-6 carbon atoms, halogen, haloalkyl of 1-6 carbon atoms and a number of halogen atoms up to the perhalo level, aryl of 6-12 carbon atoms, and heteroaryl of 2-11 carbon atoms and 1-3 heteroatoms selected from the group consisting of N, S and O or
T
may form, together with a carbon atom adjacent to a carbon atom to which it is attached, a fused ring of 6-9 carbon atoms and 4-14 hydrogen atoms;
t
is 1-5;
R²
is selected from the group consisting of alkyl of 2-10 carbon atoms, haloalkyl of 1-10 carbon atoms and a number of halogen atoms up to the perhalo level, cycloalkyl of 3-12 carbon atoms, heterocycloalkyl of 4-7 carbon atoms and 1-3 heteroatoms selected from the group consisting of N, S and O alkenyl of 2-10 carbon atoms, cycloalkenyl of 5-12 carbon atoms and containing 1-3 rings, and alkynyl of 3-10 carbon atoms;
G
is a selected from the group consisting of hydrogen, nitro, nitrile, halogen, OH, OR⁴, ═O, haloalkyl of 1-4 carbon atoms and a number of halogen atoms up to the perhalo level, alkyl of 1-4 carbon atoms, alkenyl of 1-4 carbon atoms, cycloalkyl of 3-7 carbon atoms, heterocycloalkyl of 3-5 carbon atoms and 1-3 heteroatoms selected from the group consisting of N, O, and S, cycloalkenyl of 5-7 carbon atoms, heterocycloalkenyl of 4-6 carbon atoms and 1-3 heteroatoms selected from the group consisting of N, O, and S, CO₂R⁴, C(O)N(R⁵)(R⁶), aryl of 6-10 carbon atoms, heteroaryl of 3-9 carbon atoms and 1-3 heteroatoms selected from the group consisting of N, O, and S, S(O)_{y}R⁷, SO₃R⁷, and SO₂N(R⁵)(R⁶);
R⁴
is selected from the group consisting of alkyl of 1-4 carbon atoms, haloalkyl of 1-4 carbon atoms and a number of halogen atoms up to the perhalo level, cycloalkyl of 3-6 carbon atoms, and halocycloalkyl of 3-6 carbon atoms;
R⁵ and R⁶
are each independently selected from the group consisting of hydrogen and alkyl of 1-5 carbon atoms;
R⁷
is selected from the group consisting of alkyl of 1-5 carbon atoms, SO₂F, CHO, OH, nitro, nitrile, halogen, OCF₃, N-oxide, O—C(R⁸)₂O, C(O)NHC(O), the carbon atoms being connected to adjacent positions on R, and C(O)C₆H₄, the carbonyl carbon and the ring carbon ortho to the carbonyl carbon being connected to adjacent positions on R;
R⁸
is selected from the group consisting of hydrogen, halogen and alkyl of 1-4 carbon atoms;
y
is 0-2
g
is 0-4, with the exception of halogen, which may be employed up to the perhalo level;
provided that when G is alkyl of 1-4 carbon atoms, alkenyl of 1-4 carbon atoms, cycloalkyl of 3-7 carbon atoms, heterocycloalkyl of 3-5 carbon atoms, cycloalkenyl of 5-7 carbon atoms, or heterocycloalkenyl of 4-6 carbon atoms, then G optionally may bear secondary substituents of halogen up to the perhalo level; and when G is aryl or heteroaryl, then G optionally may bear secondary substituents independently selected from the group consisting of alkyl of 1-4 carbon atoms and halogen, the number of said secondary substituents being up to 3 for alkyl moieties, and up to the perhalo level for halogen;
x
forms, together with the nitrogen atom and carbon atom to which it is attached, a polycyclic ring structure of containing 3-4 rings, wherein each ring contains 3-8 carbon atoms and may optionally be substituted with one or more of alkyl of 1-6 carbon atoms or alkenyl of 2-6 carbon atoms, or
X is alkyl of 3-7 carbon atoms or alkenyl of 3-7 carbon atoms;
R¹⁰, R¹¹ and R¹²
(i) are each independently selected from the group consisting of hydrogen, haloalkyl of 1-10 carbon atoms and a number of halogen atoms up to the perhalo level, alkyl of 1-10 carbon atoms, cycloalkyl of 3-12 carbon atoms, alkenyl of 2-10 carbon atoms, cycloalkenyl of 5-7 carbon atoms, and alkynyl of 3-10 carbon atoms, or
(ii) R¹⁰ and R¹¹ are each independently selected from the group consisting of hydrogen, haloalkyl of 1-10 carbon atoms and a number of halogen atoms up to the perhalo level, alkyl of 1-10 carbon atoms, cycloalkyl of 3-12 carbon atoms, alkenyl of 2-10 carbon atoms, cycloalkenyl of 5-7 carbon atoms, and alkynyl of 3-10 carbon atoms and R¹² forms, together with the carbon atom to which it is attached, a Spiro ring of 3-6 carbon atoms, or
(iii) R¹⁰ and R¹¹ are each independently selected from the group consisting of hydrogen, haloalkyl of 1-10 carbon atoms and a number of halogen atoms up to the perhalo level, alkyl of 1-10 carbon atoms, cycloalkyl of 3-12 carbon atoms, alkenyl of 2-10 carbon atoms, cycloalkenyl of 5-7 carbon atoms, and alkynyl of 3-10 carbon atoms and R¹² forms, together with the carbon atom adjacent to the carbon atom to which it is attached, a fused ring of 3-7 carbon atoms and 4-14 hydrogen atoms, or
(iv) R¹⁰ and R¹¹ are each independently-selected from the group consisting of hydrogen, haloalkyl of 1-10 carbon atoms and a number of halogen atoms up to the perhalo level, alkyl of 1-10 carbon atoms, cycloalkyl of 3-12 carbon atoms, alkenyl of 2-10 carbon atoms, cycloalkenyl of 5-7 carbon atoms, and alkynyl of 3-10 carbon atoms and R¹² forms, together with the carbon atom that is 2-4 carbon atoms away from the carbon atom to which it is attached, a fused ring of 3-7 carbon atoms and 4-14 hydrogen atoms;
with the proviso that when X is alkyl of 3-4 carbon atoms and R¹⁰, R¹¹ and R¹² are all hydrogen:
t is 2-5;
at least one of T is 4-nitro or 4-nitrile and at least one other T is 2-alkyl, 2-halogen or 2-trifluoromethyl;
and R¹ is phenyl;
and with the further proviso that when X is alkyl of 3-7 carbon atoms or alkenyl of 3-7 carbon atoms and R¹⁰, R¹¹ and R¹² are each independently selected from the group consisting of hydrogen, haloalkyl of 1-10 carbon atoms and a number of halogen atoms up to the perhalo level, alkyl of 1-10 carbon atoms, cycloalkyl of 3-12 carbon atoms, alkenyl of 2-10 carbon atoms, cycloalkenyl of 5-7 carbon atoms, and alkynyl of 3-10 carbon atoms, then at least one of T is nitro, nitrile, trifluoromethyl or halogen;
and pharnaceutically acceptable salts thereof

2. The compound of claim 1, wherein
R¹
is selected from the group consisting of aryl of 6-12 carbon atoms and heteroaryl of 6-11 carbon atoms and 1-3 heteroatoms selected from the group consisting of N, S and O;
T
is selected from the group consisting of hydrogen, nitro, nitrile, alkyl of 1-6 carbon atoms, halogen, haloalkyl of 1-6 carbon atoms and a number of halogen atoms up to the perhalo level, aryl of 6-12 carbon atoms, and heteroaryl of 2-11 carbon atoms and 1-3 heteroatoms selected from the group consisting of N, S and O with the proviso that at least one of T is nitro, nitrile, trifluoromethyl or halogen, or
T
may form, together with a carbon atom adjacent to a carbon atom to which it is attached, a fused ring of 6-9 carbon atoms and 4-14 hydrogen atoms;
t
is 1-5;
R²
is selected from the group consisting of alkyl of 2-10 carbon atoms, haloalkyl of 1-10 carbon atoms and a number of halogen atoms up to the perhalo level, cycloalkyl of 3-12 carbon atoms, heterocycloalkyl of 4-7 carbon atoms and 1-3 heteroatoms selected from the group consisting of N, S and O alkenyl of 2-10 carbon atoms, cycloalkenyl of 5-12 carbon atoms and containing 1-3 rings, and alkynyl of 3-10 carbon atoms;
G
is selected from the group consisting of hydrogen, nitro, nitrile, halogen, OH, OR⁴, ═O, alkyl of 1-4 carbon atoms, alkenyl of 1-4 carbon atoms, cycloalkyl of 3-7 carbon atoms, heterocycloalkyl of 3-5 carbon atoms and 1-3 heteroatoms selected from the group consisting of N, O, and S, cycloalkenyl of 5-7 carbon atoms, heterocycloalkenyl of 4-6 carbon atoms and 1-3 heteroatoms selected from the group consisting of N, O, and S, CO₂R⁴, C(O)N(R⁵)(R⁶), aryl of 6-10 carbon atoms, heteroaryl of 3-9 carbon atoms and 1-3 heteroatoms selected from the group consisting of N, O, and S, S(O)_{y}R⁷, SO₃R⁷, and SO₂N(R⁵)(R⁶);
R⁴
is selected from the group consisting of alkyl of 1-4 carbon atoms, haloalkyl of 1-4 carbon atoms and a number of halogen atoms up to the perhalo level, cycloalkyl of 3-6 carbon atoms, and balocycloalkyl of 3-6 carbon atoms;
R⁵ and R ⁶
are each independently selected from the group consisting of hydrogen and alkyl of 1-5 carbon atoms;
R⁷
is selected from the group consisting of alkyl of 1-5 carbon atoms, SO₂F, CHO, OH, nitro, nitrile, halogen, OCF₃, N-oxide, O—C(R⁸)₂O, C(O)NHC(O), the carbon atoms being connected to adjacent positions on R, and C(O)C₆H₄, the carbonyl carbon and the ring carbon ortho to the carbonyl carbon being connected to adjacent positions on R;
R⁸
is selected from the group consisting of hydrogen, halogen and alkyl of 1-4 carbon atoms;
y
is 0-2
g
is 0-4, with the exception of halogen, which may be employed up to the perhalo level;
provided that when G is alkyl of 1-4 carbon atoms, alkenyl of 1-4 carbon atoms, cycloalkyl of 3-7 carbon atoms, heterocycloalkyl of 3-5 carbon atoms, cycloalkenyl of 5-7 carbon atoms, or heterocycloalkenyl of 4-6 carbon atoms, then G optionally may bear secondary substituents of halogen up to the perhalo level; and when G is aryl or heteroaryl, then G optionally may bear secondary substituents independently selected from the group consisting of alkyl of 1-4 carbon atoms and halogen, the number of said secondary substituents being up to 3 for alkyl moieties, and up to the perhalo level for halogen;
X is alkyl of 3-7 carbon atoms or alkenyl of 3-7 carbon atoms;
R¹⁰, R¹¹ and R¹²
are each independently selected from the group consisting of hydrogen, haloalkyl of 1-10 carbon atoms and a number of halogen atoms up to the perhalo level, alkyl of 1-10 carbon atoms, cycloalkyl of 3-12 carbon atoms, alkenyl of 2-10 carbon atoms, cycloalkenyl of 5-7 carbon atoms, and alkcnyl of 3-10 carbon atoms;
with the proviso that when X is alkyl of 3-4 carbon atoms and R¹⁰, R¹¹ and R¹² are all hydrogen:
t is 2-5;
at least one of T is 4-nitro or 4-nitrile and at least one other T is 2-alkyl, 2-halogen or 2-trifluoromethyl;
and R¹ is phenyl;
and phannaceutically acceptable salts thereof.

3. The compound of claim 2, wherein
R¹
is selected from the group consisting of aryl of 6-12 carbon atoms and heteroaryl of 2-11 carbon atoms and 1-3 heteroatoms selected from the group consisting of N, S and O;
T
is selected from the group consisting of hydrogen, alkyl of 1-4 carbon atoms, halogen, haloalkyl of 1-4 carbon atoms and 1-3 halo atoms, nitro, and nitrile, with the proviso that at least one of T is nitro, nitrile, trifluoromethyl or halogen;
t
is 1-3;
R²
is selected from the group consisting of alkyl of 2-10 carbon atoms, haloalkyl of 1-10 carbon atoms and a number of halogen atoms up to the perhalo level, cycloalkyl of 3-12 carbon atoms, heterocycloalkyl of 4-7 carbon atoms and 1-3 heteroatoms selected from the group consisting of N, S and O alkenyl of 2-10 carbon atoms, cycloalkenyl of 5-12 carbon atoms and containing 1-3 rings, and alkynyl of 3-10 carbon atoms;
G
is selected from the group consisting of hydrogen, nitro, nitrile, halogen, OH, OR⁴, alkyl of 1-4 carbon atoms, alkenyl of 1-4 carbon atoms, cycloalkyl of 3-7 carbon atoms, cycloalkenyl of 5-7 carbon atoms, CO₂R⁴, aryl of 6-10 carbon atoms, and heteroaryl of 3-9 carbon atoms and 1-3 heteroatoms selected from the group consisting of N, O, and S;
R⁴
is selected from the group consisting of alkyl of 1-4 carbon atoms, haloalkyl of 1-4 carbon atoms and a number of halogen atoms up to the perhalo level, cycloalkyl of 3-6 carbon atoms, and halocycloalkyl of 3-6 carbon atoms;
g
is 0-4, with the exception of halogen, which may be employed up to the perhalo level;
provided that when G is alkyl of 1-4 carbon atoms, alkenyl of 1-4 carbon atoms, cycloalkyl of 3-7 carbon atoms, heterocycloalkyl of 3-5 carbon atoms, cycloalkenyl of 5-7 carbon atoms, or heterocycloalkenyl of 4-6 carbon atoms, then G optionally may bear secondary substituents of halogen up to the perhalo level; and when G is aryl or heteroaryl, then G optionally may bear secondary substituents independently selected from the group consisting of alkyl of 1-4 carbon atoms and halogen, the number of said secondary substituents being up to 3 for alkyl moieties, and up to the perhalo level for halogen;
X is alkyl of 3-7 carbon atoms or alkenyl of 3-7 carbon atoms;
R¹⁰, R¹¹ and R¹²
are each independently selected from the group consisting of haloalkyl of 1-10 carbon atoms and a number of halogen atoms up to the perhalo level, alkyl of 1-10 carbon atoms, cycloaLkyl of 3-12 carbon atoms, alkenyl of 2-10 carbon atoms, cycloalkenyl of 5-7 carbon atoms, and alkynyl of 3-10 carbon atoms;
and pharmaceutically acceptable salts thereof.

4. The compound of claim 3, wherein
R¹
is aryl of 6-12 carbon atoms;
R²
is selected from the group consisting of aLkyl of 2-10 carbon atoms, haloalkyl of 1-10 carbon atoms and a number of halogen atoms up to the perhalo level, cycloalkyl of 3-12 carbon atoms, heterocycloalkyl of 4-7 carbon atoms and 1-3 heteroatoms selected from the group consisting of N, S and O alkenyl of 2-10 carbon atoms, cycloalkenyl of 5-12 carbon atoms and containing 1-3 rings, and alkynyl of 3-10 carbon atoms;
G
is hydrogen;
g
is 0-4;
T
is selected from the group consisting of nitro, nitrile, trifluoromethyl and halogen;
t
is 1-2;
X is alkyl of 3-7 carbon atoms or alkenyl of 3-7 carbon atoms;
R¹⁰, R¹¹ and R¹²
haloalkyl of 1-10 carbon atoms and a number of halogen atoms up to the perhalo level, alkyl of 1-10 carbon atoms, cycloalkyl of 3-12 carbon atoms, alkenyl of 2-10 carbon atoms, cycloalkenyl of 5-7 carbon atoms, and alkynyl of 3-10 carbon atoms;
and pharmaceutically acceptable salts thereof.

5. The compound of claim 2 selected from the group consisting of: 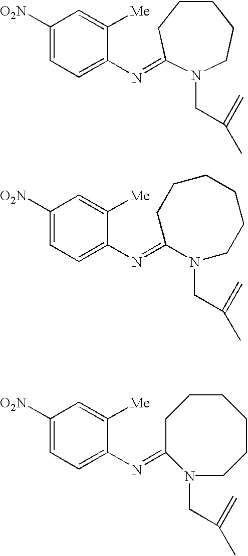
and pharmaceutically acceptable salts thereof.

6. The compound of claim 1, wherein
R¹
is selected from the group consisting of aryl of 6-12 carbon atoms and heteroaryl of 2-11 carbon atoms and 1-3 heteroatoms selected from the group consisting of N, S and O;
T
is selected from the group consisting of hydrogen, nitro, nitrile, alkyl of 1-6 carbon atoms, halogen, haloalkyl of 1-6 carbon atoms and a number of halogen atoms up to the perhalo level, aryl of 6-12 carbon atoms, and heteroaryl of 2-11 carbon atoms and 1-3 heteroatoms selected from the group consisting of N, S and O or
T
may form, together with a carbon atom adjacent to a carbon atom to which it is attached, a fused ring of 6-9 carbon atoms and 4-14 hydrogen atoms;
t
is 1-5;
R²
is selected from the group consisting of alkyl of 2-10 carbon atoms, haloalkyl of 1-10 carbon atoms and a number of halogen atoms up to the perhalo level, cycloalkyl of 3-12 carbon atoms, heterocycloalkyl of 4-7 carbon atoms and 1-3 heteroatoms selected from the group consisting of N, S and O alkenyl of 2-10 carbon atoms, cycloalkenyl of 5-12 carbon atoms and containing 1-3 rings, and alkynyl of 3-10 carbon atoms;
G
is selected from the group consisting of hydrogen, nitro, nitrile, halogen, OH, OR⁴, ═O, alkyl of 1-4 carbon atoms, alkenyl of 1-4 carbon atoms, cycloalkyl of 3-7 carbon atoms, heterocycloalkyl of 3-5 carbon atoms and 1-3 heteroatoms selected from the group consisting of N, O, and S, cycloalkenyl of 5-7 carbon atoms, heterocycloalkenyl of 4-6 carbon atoms and 1-3 heteroatoms selected from the group consisting of N, O, and S, CO₂R⁴, C(O)N(R⁵)(R⁶), aryl of 6-10 carbon atoms, heteroaryl of 3-9 carbon atoms and 1-3 heteroatoms selected from the group consisting of N, O, and S, S(O)_{y}R⁷, SO₃R⁷, and SO₂N(R⁵)(R⁶);
R⁴
is selected from the group consisting of alkyl of 1-4 carbon atoms, haloalkyl of 1-4 carbon atoms and a number of halogen atoms up to the perhalo level, cycloalkyl of 3-6 carbon atoms, and halocycloalkyl of 3-6 carbon atoms;
R⁵ and R⁶
are each independently selected from the group consisting of hydrogen and alkyl of 1-5 carbon atoms;
R⁷
is selected from the group consisting of alkyl of 1-5 carbon atoms, SO₂F, CHO, OH, nitro, nitrile, halogen, OCF₃, N-oxide, O—C(R⁸)₂O, C(O)NHC(O), the carbon atoms being connected to adjacent positions on R, and C(O)C₆H₄, the carbonyl carbon and the ring carbon ortho to the carbonyl carbon being connected to adjacent positions on R;
R⁸
is selected from the group consisting of hydrogen, halogen and alkyl of 1-4 carbon atoms;
y
is 0-2
g
is 0-4, with the exception of halogen, which may be employed up to the perhalo level; provided that when G is alkyl of 1-4 carbon atoms, alkenyl of 1-4 carbon atoms, cycloalkyl of 3-7 carbon atoms, heterocycloalkyl of 3-5 carbon atoms, cycloalkenyl of 5-7 carbon atoms, or heterocycloalkenyl of 4-6 carbon atoms, then G optionally may bear secondary substituents of halogen up to the perhalo level; and when G is aryl or heteroaryl, then G optionally may bear secondary substituents independently selected from the group consisting of alkyl of 1-4 carbon atoms and halogen, the number of said secondary substituents being up to 3 for allyl moieties, and up to the perhalo level for halogen;
X
forms, together with the nitrogen atom and carbon atom to which it is attached, a polycyclic ring structure of containing 3-4 rings, wherein each ring contains 3-8 carbon atoms and may optionally be substituted with one or more of alkyl of 1-6 carbon atoms or alkenyl of 2-6 carbon atoms, or
X is alkyl of 3-7 carbon atoms or alkenyl of 3-7 carbon atoms;
R¹⁰, R¹¹ and R¹²
(i) R¹⁰ and R¹¹ are each independently selected from the group consisting of hydrogen, haloalkyl of 1-10 carbon atoms and a number of halogen atoms up to the perhalo level, alkyl of 1-10 carbon atoms, cycloalkyl of 3-12 carbon atoms, alkenyl of 2-10 carbon atoms, cycloalkenyl of 5-7 carbon atoms, and alkynyl of 3-10 carbon atoms and R¹² forms, together with the carbon atom to which it is attached, a spiro ring of 3-6 carbon atoms, or
(ii) R¹⁰ and R¹¹ are each independently selected from the group consisting of hydrogen, haloalkyl of 1-10 carbon atoms and a number of halogen atoms up to the perhalo level, alkyl of 1-10 carbon atoms, cycloalkyl of 3-12 carbon atoms, alkenyl of 2-10 carbon atoms, cycloalkenyl of 5-7 carbon atoms, and alkynyl of 3-10 carbon atoms and R¹² forms, together with the carbon atom adjacent to the carbon atom to which it is attached, a fused ring of 3-7 carbon atoms and 4-14 hydrogen atoms, or
(iii) R¹⁰ and R¹¹ are each independently selected from the group consisting of hydrogen, haloalkyl of 1-10 carbon atoms and a number of halogen atoms up to the perhalo level, alkyl of 1-10 carbon atoms, cycloalkyl of 3-12 carbon atoms, alkenyl of 2-10 carbon atoms, cycloalkenyl of 5-7 carbon atoms, and alkynyl of 3-10 carbon atoms and R¹² forms, together with the carbon atom that is 2-4 carbon atoms away from the carbon atom to which it is attached, a fused ring of 3-7 carbon atoms and 4-14 hydrogen atoms;
and pharmaceutically acceptable salts thereof.

7. The compound of claim 6, wherein
R¹
is selected from the group consisting of aryl of 6-12 carbon atoms and heteroaryl of 2-11 carbon atoms and 1-3 heteroatoms selected from the group consisting of N, S and O;
T
is selected from the group consisting of hydrogen, nitro, nitrile, alkyl of 1-6 carbon atoms, halogen, haloalkyl of 1-6 carbon atoms and a number of halogen atoms up to the perhalo level, aryl of 6-12 carbon atoms, and heteroaryl of 2-11 carbon atoms and 1-3 heteroatoms selected from the group consisting of N, S and O or
T
may form, together with a carbon atom adjacent to a carbon atom to which it is attached, a fused ring of 6-9 carbon atoms and 4-14 hydrogen atoms;
t
is 1-5;
R²
is selected from the group consisting of alkyl of 2-10 carbon atoms, haloalkyl of 1-10 carbon atoms and a number of halogen atoms up to the perhalo level, cycloalkyl of 3-12 carbon atoms, heterocycloalkyl of 4-7 carbon atoms and 1-3 heteroatoms selected from the group consisting of N, S and O alkenyl of 2-10 carbon atoms, cycloalkenyl of 5-12 carbon atoms and containing 1-3 rings, and alkynyl of 3-10 carbon atoms;
G
is selected from the group consisting of hydrogen, nitro, nitrile, halogen, OH, OR⁴, ═O, alkyl of 1-4 carbon atoms, alkenyl of 1-4 carbon atoms, cycloalkyl of 3-7 carbon atoms, heterocycloalkyl of 3-5 carbon atoms and 1-3 heteroatoms selected from the group consisting of N, O, and S, cycloalkenyl of 5-7 carbon atoms, heterocycloalkenyl of 4-6 carbon atoms and 1-3 heteroatoms selected from the group consisting of N, O, and S, CO₂R⁴, C(O)N(R⁵)(R⁶), aryl of 6-10 carbon atoms, heteroaryl of 3-9 carbon atoms and 1-3 heteroatoms selected from the group consisting of N, O, and S, S(O)_{y}R⁷, SO₃R⁷, and SO₂N(R⁵)(R⁶);
R⁴
is selected from the group consisting of alkyl of 1-4 carbon atoms, haloalkyl of 1-4 carbon atoms and a number of halogen atoms up to the perhalo level, cycloalkyl of 3-6 carbon atoms, and halocycloalkyl of 3-6 carbon atoms;
R⁵ and R⁶
are each independently selected from the group consisting of hydrogen and alkyl of 1-5 carbon atoms;
R⁷
is selected from the group consisting of alkyl of 1-5 carbon atoms, SO₂F, CHO, OH, nitro, nitrile, halogen, OCF₃, N-oxide, O—C(R⁸)₂O, C(O)NHC(O), the carbon atoms being connected to adjacent positions on R, and C(O)C₆H₄, the carbonyl carbon and the ring carbon ortho to the carbonyl carbon being connected to adjacent positions on R;
R⁸
is selected from the group consisting of hydrogen, halogen and alkyl of 1-4 carbon atoms;
y
is 0-2
g
is 0-4, with the exception of halogen, which may be employed up to the perhalo level;
provided that when G is alkyl of 1-4 carbon atoms, alkenyl of 1-4 carbon atoms, cycloalkyl of 3-7 carbon atoms, heterocycloalkyl of 3-5 carbon atoms, cycloalkenyl of 5-7 carbon atoms, or heterocycloalkenyl of 4-6 carbon atoms, then G optionally may bear secondary substituents of halogen up to the perhalo level; and when G is aryl or heteroaryl, then G optionally may bear secondary substituents independently selected from the group consisting of alkyl of 1-4 carbon atoms and halogen, the number of said secondary substituents being up to 3 for alkyl moieties, and up to the perhalo level for halogen;
X is alkyl of 3-7 carbon atoms or alkenyl of 3-7 carbon atoms;
R¹⁰, R¹¹ and R¹²
(i) R¹⁰ and R¹¹ are each independently selected from the group consisting of hydrogen, haloalkyl of 1-10 carbon atoms and a number of halogen atoms up to the perhalo level, alkyl of 1-10 carbon atoms, cycloalkyl of 3-12 carbon atoms, alkenyl of 2-10 carbon atoms, cycloalkenyl of 5-7 carbon atoms, and alkynyl of 3-10 carbon atoms and R¹² forms, together with the carbon atom to which it is attached, a spiro ring of 3-6 carbon atoms, or
(ii) R¹⁰ and R¹¹ are each independently selected from the group consisting of hydrogen, haloalkyl of 1-10 carbon atoms and a number of halogen atoms up to the perhalo level, alkyl of 1-10 carbon atoms, cycloalkyl of 3-12 carbon atoms, alkenyl of 2-10 carbon atoms, cycloalkenyl of 5-7 carbon atoms, and alkynyl of 3-10 carbon atoms and R¹² forms, together with the carbon atom adjacent to the carbon atom to which it is attached, a fused ring of 3-7 carbon atoms and 4-14 hydrogen atoms, or
(iii) R¹⁰ and R¹¹ are each independently selected from the group consisting of hydrogen, haloalkyl of 1-10 carbon atoms and a number of halogen atoms up to the perhalo level, alkyl of 1-10 carbon atoms, cycloalkyl of 3-12 carbon atoms, alkenyl of 2-10 carbon atoms, cycloalkenyl of 5-7 carbon atoms, and alkynyl of 3-10 carbon atoms and R¹² forms, together with the carbon atom that is 2-4 carbon atoms away from the carbon atom to which it is attached, a fused ring of 3-7 carbon atoms and 4-14 hydrogen atoms;
and pharmaceutically acceptable salts thereof.

8. The compound of claim 7, wherein
R¹
is selected from the group consisting of aryl of 6-12 carbon atoms and heteroaryl of 2-11 carbon atoms and 1-3 heteroatoms selected from the group consisting of N, S and O;
T
is selected from the group consisting of hydrogen, alkyl of 1-4 carbon atoms, halogen, haloalkyl of 1-4 carbon atoms and 1-3 halo atoms, nitro, and nitrile;
t
is 1-3;
R²
is selected from the group consisting of alkyl of 2-10 carbon atoms, haloalkyl of 1-10 carbon atoms and a number of halogen atoms up to the perhalo level, cycloalkyl of 3-12 carbon atoms, heterocycloalkyl of 4-7 carbon atoms and 1-3 heteroatoms selected from the group consisting of N, S and O alkenyl of 2-10 carbon atoms, cycloalkenyl of 5-12 carbon atoms and containing 1-3 rings, and alkynyl of 3-10 carbon atoms;
G
is selected from the group consisting of hydrogen, nitro, nitrile, halogen, OH, OR⁴, alkyl of 1-4 carbon atoms, alkenyl of 1-4 carbon atoms, cycloalkyl of 3-7 carbon atoms, cycloalkenyl of 5-7 carbon atoms, C0₂R⁴, aryl of 6-10 carbon atoms, and heteroaryl of 3-9 carbon atoms and 1-3 heteroatoms selected from the group consisting of N, O, and S;
R⁴
is selected from the group consisting of alkyl of 1-4 carbon atoms, haloalkyl of 1-4 carbon atoms and a number of halogen atoms up to the perhalo level, cycloalkyl of 3-6 carbon atoms, and halocycloalkyl of 3-6 carbon atoms;
g
is 0-4, with the exception of halogen, which may be employed up to the perhalo level;
provided that when G is alkyl of 1-4 carbon atoms, alkenyl of 1-4 carbon atoms, cycloalkyl of 3-7 carbon atoms, heterocycloalkyl of 3-5 carbon atoms, cycloalkenyl of 5-7 carbon atoms, or heterocycloalkenyl of 4-6 carbon atoms, then G optionally may bear secondary substituents of halogen up to the perhalo level; and when G is aryl or heteroaryl, then G optionally may bear secondary substituents independently selected from the group consisting of alkyl of 1-4 carbon atoms and halogen, the number of said secondary substituents being up to 3 for alkyl moieties, and up to the perhalo level for halogen;
X is alkyl of 3-7 carbon atoms or alkenyl of 3-7 carbon atoms;
R¹⁰, R¹¹ and R¹²
(i) R¹⁰ and R¹¹ are each independently selected from the group consisting of hydrogen, haloalkyl of 1-10 carbon atoms and a number of halogen atoms up to the perhalo level, alkyl of 1-10 carbon atoms, cycloalkyl of 3-12 carbon atoms, alkenyl of 2-10 carbon atoms, cycloalkenyl of 5-7 carbon atoms, and alkynyl of 3-10 carbon atoms and R¹² forms, together with the carbon atom to which it is attached, a Spiro ring of 3-6 carbon atoms, or
(ii) R¹⁰ and R¹¹ are each independently selected from the group consisting of hydrogen, haloalkyl of 1-10 carbon atoms and a number of halogen atoms up to the perhalo level, alkyl of 1-10 carbon atoms, cycloalkyl of 3-12 carbon atoms, alkenyl of 2-10 carbon atoms, cycloalkenyl of 5-7 carbon atoms, and alkynyl of 3-10 carbon atoms and R¹² forms, together with the carbon atom adjacent to the carbon atom to which it is attached, a fused ring of 3-7 carbon atoms and 4-14 hydrogen atoms, or
(iii) R¹⁰ and R¹¹ are each independently selected from the group consisting of hydrogen, haloalkyl of 1-10 carbon atoms and a number of halogen atoms up to the perhalo level, alkyl of 1-10 carbon atoms, cycloalkyl of 3-12 carbon atoms, alkenyl of 2-10 carbon atoms, cycloalkenyl of 5-7 carbon atoms, and alkynyl of 3-10 carbon atoms and R¹² forms, together with the carbon atom that is 2-4 carbon atoms away from the carbon atom to which it is attached, a fused ring of 3-7 carbon atoms and 4-14 hydrogen atoms;
and pharmaceutically acceptable salts thereof.

9. The compound of claim 8, wherein
R¹
is aryl of 6-12 carbon atoms;
R²
is selected from the group consisting of alkyl of 2-10 carbon atoms, haloalkyl of 1-10 carbon atoms and a number of halogen atoms up to the perhalo level, cycloalkyl of 3-12 carbon atoms, heterocycloalkyl of 4-7 carbon atoms and 1-3 heteroatoms selected from the group consisting of N, S and O alkenyl of 2-10 carbon atoms, cycloalkenyl of 5-12 carbon atoms and containing 1-3 rings, and alkynyl of 3-10 carbon atoms;
G
is hydrogen;
g
is 0-4;
T
is selected from the group consisting of hydrogen, alkyl of 1-4 carbon atoms, halogen, habalkyl of 1-4 carbon atoms and 1-3 halo atoms, nitro, and nitrile;
t
is 1-2;
X is alkyl of 3-7 carbon atoms or alkenyl of 3-7 carbon atoms;
R¹⁰, R¹¹ and R¹²
(i) R¹⁰ and R¹¹ are each independently selected from the group consisting of hydrogen, haloalkyl of 1-10 carbon atoms and a number of halogen atoms up to the perhalo level, alkyl of 1-10 carbon atoms, cycloalkyl of 3-12 carbon atoms, alkenyl of 2-10 carbon atoms, cycloalkenyl of 5-7 carbon atoms, and alkynyl of 3-10 carbon atoms and R¹² forms, together with the carbon atom to which it is attached, a spiro ring of 3-6 carbon atoms, or
(ii) R¹⁰ and R¹¹ are each independently selected from the group consisting of hydrogen, haloalkyl of 1-10 carbon atoms and a number of halogen atoms up to the perhalo level, alkyl of 1-10 carbon atoms, cycloalkyl of 3-12 carbon atoms, alkenyl of 2-10 carbon atoms, cycloalkenyl of 5-7 carbon atoms, and alkynyl of 3-10 carbon atoms and R¹² forms, together with the carbon atom adjacent to the carbon atom to which it is attached, a fused ring of 3-7 carbon atoms and 4-14 hydrogen atoms, or
(iii) R¹⁰ and R¹¹ are each independently selected from the group consisting of hydrogen, haloalkyl of 1-10 carbon atoms and a number of halogen atoms up to the perhalo level, alkyl of 1-10 carbon atoms, cycloalkyl of 3-12 carbon atoms, alkenyl of 2-10 carbon atoms, cycloalkenyl of 5-7 carbon atoms, and alkynyl of 3-10 carbon atoms and R ² forms, together with the carbon atom that is 2-4 carbon atoms away from the carbon atom to which it is attached, a fused ring of 3-7 carbon atoms and 4-14 hydrogen atoms;
and pharmaceutically acceptable salts thereof

10. The compound of claim 7, selected from the group consisting of: 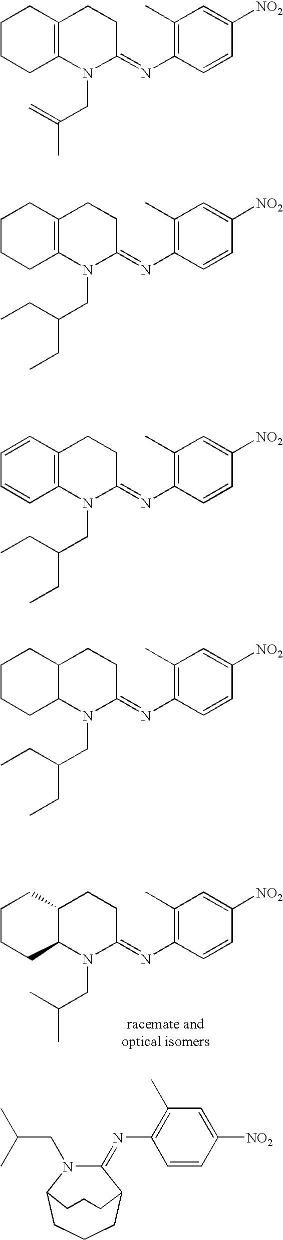 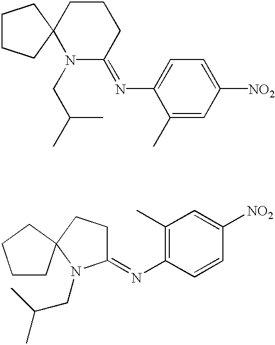
and pharmaceutically acceptable salts thereof.

11. The compound of claim 6, wherein
R¹
is selected from the group consisting of aryl of 6-12 carbon atoms and heteroaryl of 2-11 carbon atoms and 1-3 heteroatoms selected from the group consisting of N, S and O;
T
is selected from the group consisting of hydrogen, nitro, nitrile, alkyl of 1-6 carbon atoms, halogen, haloalkyl of 1-6 carbon atoms and a number of halogen atoms up to the perhalo level, aryl of 6-12 carbon atoms, and heteroaryl of 2-11 carbon atoms and 1-3 heteroatoms selected from the group consisting of N, S and O or
T
may form, together with a carbon atom adjacent to a carbon atom to which it is attached, a fused ring of 6-9 carbon atoms and 4-14 hydrogen atoms;
t
is 1-5;
R²
is selected from the group consisting of alkyl of 2-10 carbon atoms, haloalkyl of 1-10 carbon atoms and a number of halogen atoms up to the perhalo level, cycloalkyl of 3-12 carbon atoms, heterocycloalkyl of 4-7 carbon atoms and 1-3 heteroatoms selected from the group consisting of N, S and O alkenyl of 2-10 carbon atoms, cycloalkenyl of 5-12 carbon atoms and containing 1-3 rings, and alkynyl of 3-10 carbon atoms;
G
is selected from the group consisting of hydrogen, nitro, nitrile, halogen, OH, OR⁴, ═O, alkyl of 1-4 carbon atoms, alkenyl of 1-4 carbon atoms, cycloalkyl of 3-7 carbon atoms, heterocycloalkyl of 3-5 carbon atoms and 1-3 heteroatoms selected from the group consisting of N, O, and S, cycloalkenyl of 5-7 carbon atoms, heterocycloalkenyl of 4-6 carbon atoms and 1-3 heteroatoms selected from the group consisting of N, O, and S, CO₂R⁴, C(O)N(R⁵)(R⁶), aryl of 6-10 carbon atoms, heteroaryl of 3-9 carbon atoms and 1-3 heteroatoms selected from the group consisting of N, O, and S, S(O)_{y}R⁷, SO₃R⁷, and SO₂N(R⁵)(R⁶);
R⁴
is selected from the group consisting of alkyl of 1-4 carbon atoms, haloalkyl of 1-4 carbon atoms and a number of halogen atoms up to the perhalo level, cycloalkyl of 3-6 carbon atoms, and halocycloalkyl of 3-6 carbon atoms;
R⁵ and R⁶
are each independently selected from the group consisting of hydrogen and alkyl of 1-5 carbon atoms;
R⁷
is selected from the group consisting of alkyl of 1-5 carbon atoms, SO₂F, CHO, OH, nitro, nitrile, halogen, OCF₃, N-oxide, O—C(R⁸)₂O, C(O)NHC(O), the carbon atoms being connected to adjacent positions on R, and C(O)C₆H₄, the carbonyl carbon and the ring carbon ortho to the carbonyl carbon being connected to adjacent positions on R;
R⁸
is selected from the group consisting of hydrogen, halogen and alkyl of 1-4 carbon atoms;
y
is 0-2
g
is 0-4, with the exception of halogen, which may be employed up to the perhalo level;
provided that when G is alkyl of 1-4 carbon atoms, alkenyl of 1-4 carbon atoms, cycloalkyl of 3-7 carbon atoms, heterocycloalkyl of 3-5 carbon atoms, cycloalkenyl of 5-7 carbon atoms, or heterocycloalkenyl of 4-6 carbon atoms, then G optionally may bear secondary substituents of halogen up to the perhalo level; and when G is aryl or heteroaryl, then G optionally may bear secondary substituents independently selected from the group consisting of alkyl of 1-4 carbon atoms and halogen, the number of said secondary substituents being up to 3 for aLkyl moieties, and up to the perhalo level for halogen;
X
forms, together with the nitrogen atom and carbon atom to which it is attached, a polycyclic ring structure of containing 3-4 rings, wherein each ring contains 3-8 carbon atoms and may optionally be substituted with one or more of alkyl of 1-6 carbon atoms or alkenyl of 2-6 carbons atoms; and pharmaceutically acceptable salts thereof.

12. The compound of claim 11, wherein
R¹
is selected from the group consisting of aryl of 6-12 carbon atoms and heteroaryl of 2-11 carbon atoms and 1-3 heteroatoms selected from the group consisting of N, S and O;
T
is selected from the group consisting of hydrogen, alkyl of 1-4 carbon atoms, halogen, haloalkyl of 1-4 carbon atoms and 1-3 halo atoms, nitro, and nitrile;
t
is 1-3;
R²
is selected from the group consisting of alkyl of 2-10 carbon atoms, haloalkyl of 1-10 carbon atoms and a number of halogen atoms up to the perhalo level, cycloalkyl of 3-12 carbon atoms, heterocycloalkyl of 4-7 carbon atoms and 1-3 heteroatoms selected from the group consisting of N, S and O alkenyl of 2-10 carbon atoms, cycloalkenyl of 5-12 carbon atoms and containing 1-3 rings, and alkynyl of 3-10 carbon atoms;
G
is selected from the group consisting of hydrogen, halogen, nitro, nitrile, OH, OR⁴, alkyl of 1-4 carbon atoms, alkenyl of 1-4 carbon atoms, cycloalkyl of 3-7 carbon atoms, cycloalkenyl of 5-7 carbon atoms, CO₂R⁴, aryl of 6-10 carbon atoms, and heteroaryl of 3-9 carbon atoms and 1-3 heteroatoms selected from the group consisting of N, O, and S;
R⁴
is selected from the group consisting of alkyl of 1-4 carbon atoms, haloalkyl of 1-4 carbon atoms and a number of halogen atoms up to the perhalo level, cycloalkyl of 3-6 carbon atoms, and halocycloalkyl of 3-6 carbon atoms;
g
is 0-4, with the exception of halogen, which may be employed up to the perhalo level; provided that when G is alkyl of 1-4 carbon atoms, alkenyl of 1-4 carbon atoms, cycloalkyl of 3-7 carbon atoms, heterocycloalkyl of 3-5 carbon atoms, cycloalkenyl of 5-7 carbon atoms, or heterocycloalkenyl of 4-6 carbon atoms, then G optionally may bear secondary substituents of halogen up to the perhalo level; and when G is aryl or heteroaryl, then G optionally may bear secondary substituents independently selected from the group consisting of alkyl of 1-4 carbon atoms and halogen, the number of said secondary substituents being up to 3 for alkyl moieties, and up to the perhalo level for halogen;
X
forms, together with the nitrogen atom and carbon atom to which it is attached, a polycyclic ring structure of containing 3-4 rings, wherein each ring contains 3-8 carbon atoms and may optionally be substituted with one or more of alkyl of 1-6 carbon atoms or alkenyl of 2-6 carbon atoms; and pharmaceutically acceptable salts thereof.

13. The compound of claim 12, wherein
R¹
is aryl of 6-12 carbon atoms;
R²
is selected from the group consisting of alkyl of 2-10 carbon atoms, haloalkyl of 1-10 carbon atoms and a number of halogen atoms up to the perhalo level, cycloalkyl of 3-12 carbon atoms, heterocycloalkyl of 4-7 carbon atoms and 1-3 heteroatoms selected from the group consisting of N, S and O alkenyl of 2-10 carbon atoms, cycloalkenyl of 5-12 carbon atoms and containing 1-3 rings, and alkynyl of 3-10 carbon atoms;
G
is hydrogen;
g
is 0-4;
T
is selected from the group consisting of hydrogen, alkyl of 1-4 carbon atoms, halogen, haloalkyl of 1-4 carbon atoms and 1-3 halo atoms, nitro, and nitrile;
t
is 1-2;
X
is 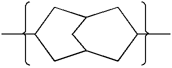
and pharmaceutically acceptable salts thereof.

14. The compound of claim 11, selected from the group consisting of: 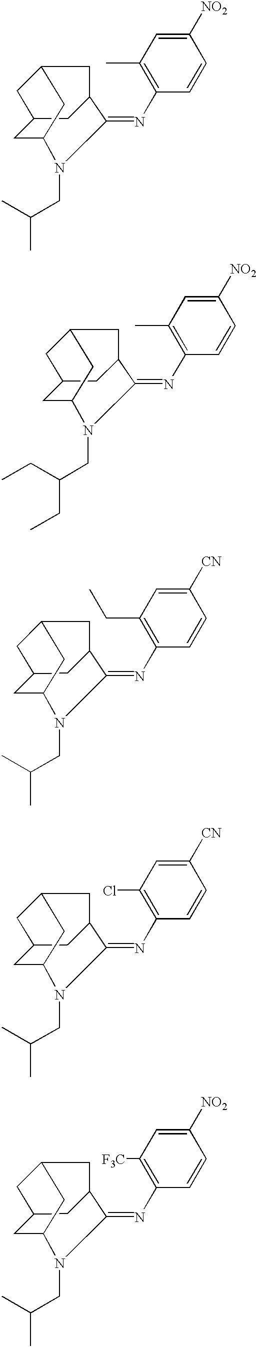 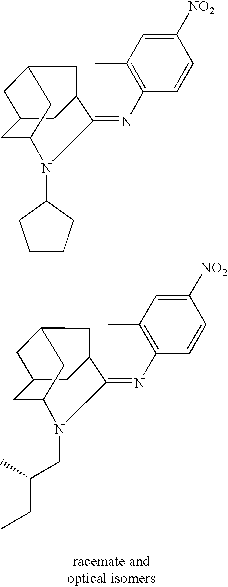
and pharmaceutically acceptable salts thereof

15. A compound of the formula (II) 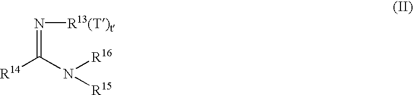
wherein
R¹³
is selected from the group consisting of aryl of 6-12 carbon atoms and 4-pyridyl;
R¹⁴
is selected from the group consisting of aryl of 6-12 carbon atoms, heteroaryl of 2-11 carbon atoms and 1-3 heteroatoms selected from the group consisting of N, S and O alkyl of 1-10 carbon atoms, cycloalkyl of 3-12 carbon atoms, heterocycloalkyl of 4-7 carbon atoms and 1-3 heteroatoms selected from the group consisting of N, S and O alkenyl of 2-10 carbon atoms, cycloalkenyl of 5-12 carbon atoms, with the proviso that said cycloalkenyl is mono-cyclic, and R¹⁷—R¹⁸;
T′
is selected from the group consisting of hydrogen, nitro, nitrile, alkyl of 1-6 carbon atoms, halogen, haloalkyl of 1-6 carbon atoms and a number of halogen atoms up to the perhalo level, aryl of 6-12 carbon atoms, and heteroaryl of 2-11 carbon atoms and 1-3 heteroatoms selected from the group consisting of N, S and O with the proviso that when R¹³ is aryl of 6-12 carbon atoms, at least one of T′ is nitro, nitrile, trifluoromethyl or halogen, or
T′
may form, together with a carbon atom adjacent to a carbon atom to which it is attached, a fused ring of 6-9 carbon atoms and 4-14 hydrogen atoms;
t′
is 1-5;
R¹⁷
is selected from the group consisting of alkyl of 1-10 carbon atoms and alkenyl of 2-10 carbon atoms;
R¹⁸
is selected from the group consisting of aryl of 6-12 carbon atoms, heteroaryl of 2-11 carbon atoms and 1-3 heteroatoms selected from the group consisting of N, S and O cycloalkyl of 3-12 carbon atoms, heterocycloalkyl of 4-7 carbon atoms and 1-3 heteroatoms selected from the group consisting of N, S and O, and cycloalkenyl of 5-12 carbon atoms;
R¹⁵ and R¹⁶
(i) are each independently selected from the group consisting of hydrogen, aryl of 6-12 carbon atoms, heteroaryl of 2-11 carbon atoms and 1-3 heteroatoms selected from the group consisting of N, S and O alkyl of 1-10 carbon atoms, cycloalkyl of 3-12 carbon atoms, heterocycloalkyl of 4-7 carbon atoms and 1-3 heteroatoms selected from the group consisting of N, S and O alkenyl of 2-10 carbon atoms, cycloalkenyl of 5-12 carbon atoms, and R¹⁹—R²⁰, such that the total number of non-hydrogen atoms in R¹⁴, R¹⁵ and R¹⁶ is greater than or equal to 9, or
(ii) are joined to form, together with the nitrogen atom to which they are attached, a 5-8 membered ring containing 4-7 carbon atoms and 1-2 heteroatoms selected from the group consisting of N, S and O which ring may optionally be substituted with R²¹ and R²², with the proviso that when R¹⁵ and R¹⁶ form a morpholine ring together with the nitrogen atom to which they are attached, said morpholine ring is substituted with at least one of R²¹ and R²²;
R¹⁹
is selected from the group consisting of alkyl of 1-10 carbon atoms, cycloalkyl of 3-12 carbon atoms, heterocycloalkyl of 4-7 carbon atoms and 1-3 heteroatoms selected from the group consisting of N, S and O alkenyl of 2-10 carbon atoms, and cycloalkenyl of 5-12 carbon atoms;
R²⁰
is selected from the group consisting of hydrogen, aryl of 6-12 carbon atoms, heteroaryl of 2-11 carbon atoms and 1-3 heteroatoms selected from the group consisting of N, S and O cycloalkyl of 3-12 carbon atoms, heterocycloalkyl of 4-7 carbon atoms and 1-3 heteroatoms selected from the group consisting of N, S and O cycloalkenyl of 5-12 carbon atoms, and R²³—R²⁴, with the proviso that when R²⁰ is phenyl, only one of R¹⁵ and R¹⁶ can be R¹⁹—R²⁰;
R²³
is selected from the group consisting of aryl of 6-12 carbon atoms and heteroaryl of 2-11 carbon atoms and 1-3 heteroatoms selected from the group consisting of N, S and O;
R²⁴
is selected from the group consisting of hydrogen, halogen, nitrile, nitro, alkyl of 1-10 carbon atoms, and haloalkyl of 1-6 carbon atoms and 1-3 halo atoms;
R²¹ and R²²
(i) are each independently selected from the group consisting of hydrogen, haloalkyl of 1-10 carbon atoms and a number of halogen atoms up to the perhalo level, alkyl of 1-10 carbon atoms, aryl of 6-12 carbon atoms, heteroaryl of 2-11 carbon atoms and 1-3 heteroatoms selected from the group consisting of N, S and O and benzimidazolinone, or
(ii) each independently forms, together with the carbon atom adjacent to the carbon atom to which it is attached, a fused ring of 3-6 carbon atoms and 4-10 hydrogen atoms, or
(iii) R²¹ is selected from the group consisting hydrogen, haloalkyl of 1-10 carbon atoms and a number of halogen atoms up to the perhalo level, alkyl of 1-10 carbon atoms, aryl of 6-12 carbon atoms, heteroaryl of 2-11 carbon atoms and 1-3 heteroatoms selected from the group consisting of N, S and O and benzimidazolinone and R²² forms, together with the carbon atom adjacent to the carbon atom to which it is attached, a fused ring of 3-6 carbon atoms and 4-10 hydrogen atoms;
and pharmaceutically acceptable salts thereof.

16. The compound of claim 15, wherein
R¹³
is selected from the group consisting of aryl of 6-12 carbon atoms and 4-pyridyl;
R¹⁴
is selected from the group consisting of aryl of 6-12 carbon atoms, heteroaryl of 2-11 carbon atoms and 1-3 heteroatoms selected from the group consisting of N, S and O alkyl of 1-10 carbon atoms, cycloalkyl of 3-12 carbon atoms, heterocycloalkyl of 4-7 carbon atoms and 1-3 heteroatoms selected from the group consisting of N, S and O alkenyl of 2-10 carbon atoms, cycloalkenyl of 5-12 carbon atoms, with the proviso that said cycloalkenyl is mono-cyclic, and R¹⁷—R¹⁸;
T′
is selected from the group consisting of hydrogen, nitro, nitrile, alkyl of 1-6 carbon atoms, halogen, haloalkyl of 1-6 carbon atoms and a number of halogen atoms up to the perhalo level, aryl of 6-12 carbon atoms, and heteroaryl of 2-11 carbon atoms and 1-3 heteroatoms selected from the group consisting of N, S and O with the proviso that when R¹³ is aryl of 6-12 carbon atoms, at least one of T′ is nitro, nitrile, trifluoromethyl or halogen, or
T′
may form, together with a carbon atom adjacent to a carbon atom to which it is attached, a fused ring of 6-9 carbon atoms and 4-14 hydrogen atoms;
t′
is 1-5;
R¹⁷
is selected from the group consisting of alkyl of 1-10 carbon atoms and alkenyl of 2-10 carbon atoms;
R¹⁸
is selected from the group consisting of aryl of 6-12 carbon atoms, heteroaryl of 2-11 carbon atoms and 1-3 heteroatoms selected from the group consisting of N, S and O cycloalkyl of 3-12 carbon atoms, heterocycloalkyl of 4-7 carbon atoms and 1-3 heteroatoms selected from the group consisting of N, S and O, and cycloalkenyl of 5-12 carbon atoms;
R¹⁵ and R¹⁶
are each independently selected from the group consisting of hydrogen, aryl of 6-12 carbon atoms, heteroaryl of 2-11 carbon atoms and 1-3 heteroatoms selected from the group consisting of N, S and O alkyl of 1-10 carbon atoms, cycloalkyl of 3-12 carbon atoms, heterocycloalkyl of 4-7 carbon atoms and 1-3 heteroatoms selected from the group consisting of N, S and O alkenyl of 2-10 carbon atoms, cycloalkenyl of 5-12 carbon atoms, and R¹⁹—R²⁰, such that the total of atoms in R¹⁴, R¹⁵ and R¹⁶ is greater than or equal to 9, or
R¹⁹
is selected from the group consisting of alkyl of 1-10 carbon atoms, cycloalkyl of 3-12 carbon atoms, heterocycloalkyl of 4-7 carbon atoms and 1-3 heteroatoms selected from the group consisting of N, S and O alkenyl of 2-10 carbon atoms, and cycloalkenyl of 5-12 carbon atoms;
R²⁰
is selected from the group consisting of hydrogen, aryl of 6-12 carbon atoms, heteroaryl of 2-11 carbon atoms and 1-3 heteroatoms selected from the group consisting of N, S and O cycloalkyl of 3-12 carbon atoms, heterocycloalkyl of 4-7 carbon atoms and 1-3 heteroatoms selected from the group consisting of N, S and O cycloalkenyl of 5-12 carbon atoms, and R²³—R²⁴, with the proviso that when R²⁰ is phenyl, only one of R¹⁵ and R¹⁶ can be R¹⁹—R²⁰;
R²³
is selected from the group consisting of aryl of 6-12 carbon atoms and heteroaryl of 2-11 carbon atoms and 1-3 heteroatoms selected from the group consisting of N, S and ;
R²⁴
is selected from the group consisting of hydrogen, halogen, nitrile, nitro, alkyl of 1-10 carbon atoms, and haloalkyl of 1-6 carbon atoms and 1-3 halo atoms;
and pharmaceutically acceptable salts thereof.

17. The compound of claim 16, wherein
R¹³
is selected from the group consisting of aryl of 6-12 carbon atoms and 4-pyridyl;
R¹⁴
is selected from the group consisting of aryl of 6-12 carbon atoms, heteroaryl of 2-11 carbon atoms and 1-3 heteroatoms selected from the group consisting of N, S and O alkyl of 1-10 carbon atoms, cycloalkyl of 3-12 carbon atoms, heterocycloalkyl of 4-7 carbon atoms and 1-3 heteroatoms selected from the group consisting of N, S and O alkenyl of 2-10 carbon atoms, cycloalkenyl of 5-12 carbon atoms, with the proviso that said cycloalkenyl is mono-cyclic, and R¹⁷—R¹⁸;
T′
is selected from the group consisting of nitro, nitrile, tifluoromethyl, and halogen;
t′
is 1-3;
R¹⁷
is selected from the group consisting of alkyl of 1-10 carbon atoms and alkenyl of 2-10 carbon atoms;
R¹⁸
is selected from the group consisting of aryl of 6-12 carbon atoms, heteroaryl of 2-11 carbon atoms and 1-3 heteroatoms selected from the group consisting of N, S and O and cycloalkyl of 3-12 carbon atoms;
R¹⁵ and R¹⁶
are each independently selected from the group consisting of hydrogen, aryl of 6-12 carbon atoms, heteroaryl of 2-11 carbon atoms and 1-3 heteroatoms selected from the group consisting of N, S and O alkyl of 1-10 carbon atoms, cycloalkyl of 3-12 carbon atoms, heterocycloalkyl of 4-7 carbon atoms and 1-3 heteroatoms selected from the group consisting of N, S and O alkenyl of 2-10 carbon atoms, cycloalkenyl of 5-12 carbon atoms, and R¹⁹—R²⁰, such that the total of atoms in R¹⁴, R¹⁵ and R¹⁶ is greater than or equal to 9;
R¹⁹
is selected from the group consisting of selected from the group consisting of alkyl of 1-10 carbon atoms, cycloalkyl of 3-12 carbon atoms, and alkenyl of 2-10 carbon atoms;
R²⁰
is selected from the group consisting of hydrogen, aryl of 6-12 carbon atoms, heteroaryl of 2-11 carbon atoms and 1-3 heteroatoms selected from the group consisting of N, S and O cycloalkyl of 3-12 carbon atoms, and R²³—R²⁴, with the proviso that when R²⁰ is phenyl, only one of R¹⁵ and R¹⁶ can be R¹⁹—R²⁰;
R²³
is selected from the group consisting of aryl of 6-12 carbon atoms and heteroaryl of 2-11 carbon atoms and 1-3 heteroatoms-selected from the group consisting of N, S and O;
R²⁴
is selected from the group consisting of hydrogen, halogen, nitrile, nitro, alkyl of 1-10 carbon atoms, and haloalkyl of 1-6 carbon atoms and 1-3 halo atoms;
and pharmaceutically acceptable salts thereof.

18. The compound of claim 17, wherein
R¹³
is aryl of 6-12 carbon atoms;
R¹⁴
is selected from the group consisting of aryl of 6-12 carbon atoms, heteroaryl of 2-11 carbon atoms and 1-3 heteroatoms selected from the group consisting of N, S and O alkyl of 1-10 carbon atoms, cycloalkyl of 3-12 carbon atoms, heterocycloalkyl of 4-7 carbon atoms and 1-3 heteroatoms selected from the group consisting of N, S and O alkenyl of 2-10 carbon atoms, cycloalkenyl of 5-12 carbon atoms, with the proviso that said cycloalkenyl is mono-cyclic, and R¹⁷—R¹⁸;
T′
is selected from the group consisting of nitro, nitrile, trifluoromethyl and halogen;
t′
is 1-3;
R¹⁷
is selected from the group consisting of alkyl of 1-10 carbon atoms and alkenyl of 2-10 carbon atoms;
R¹⁸
is selected from the group consisting of aryl of 6-12 carbon atoms, heteroaryl of 2-11 carbon atoms and 1-3 heteroatoms selected from the group consisting of N, S and O and cycloalkyl of 3-12 carbon atoms;
R¹⁵ and R¹⁶
are each independently selected from the group consisting of hydrogen, aryl of 6-12 carbon atoms, cycloalkyl of 3-12 carbon atoms, alkenyl of 2-10 carbon atoms, and R¹⁹—R²⁰, such that the total of atoms in R¹⁴, R¹⁵ and R¹⁶ is greater than or equal to 9;
R¹⁹
is alkyl of 1-10 carbon atoms;
R²⁰
is selected from the group consisting of hydrogen, aryl of 6-12 carbon atoms, heteroaryl of 2-11 carbon atoms and 1-3 heteroatoms selected from the group consisting of N, S and O cycloalkyl of 3-12 carbon atoms, and R²³—R²⁴, with the proviso that when R²⁰ is phenyl, only one of R¹⁵ and R¹⁶ can be R¹⁹—R²⁰;
R²³
is selected from the group consisting of aryl of 6-12 carbon atoms and heteroaryl of 2-11 carbon atoms and 1-3 heteroatoms selected from the group consisting of N, S and O;
R²⁴
is selected from the group consisting of hydrogen, halogen, nitrile, nitro, alkyl of 1-10 carbon atoms, and haloalkyl of 1-6 carbon atoms and 1-3 halo atoms; and pharmaceutically acceptable salts thereof

19. The compound of claim 16, selected from the group consisting of: 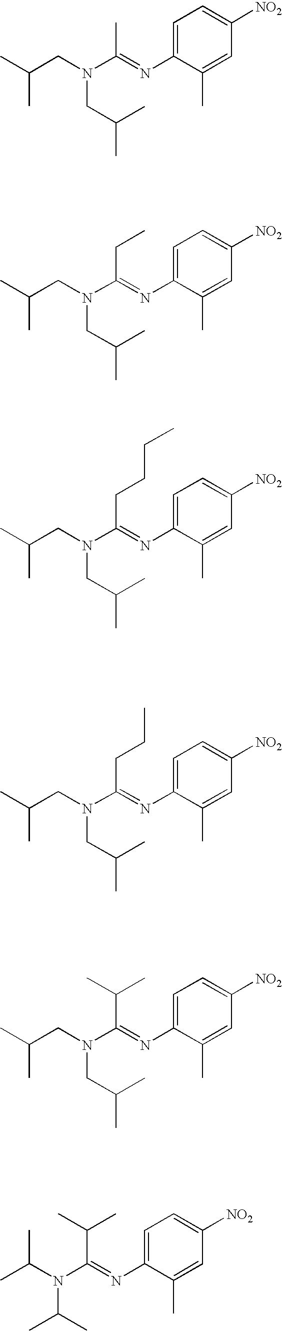 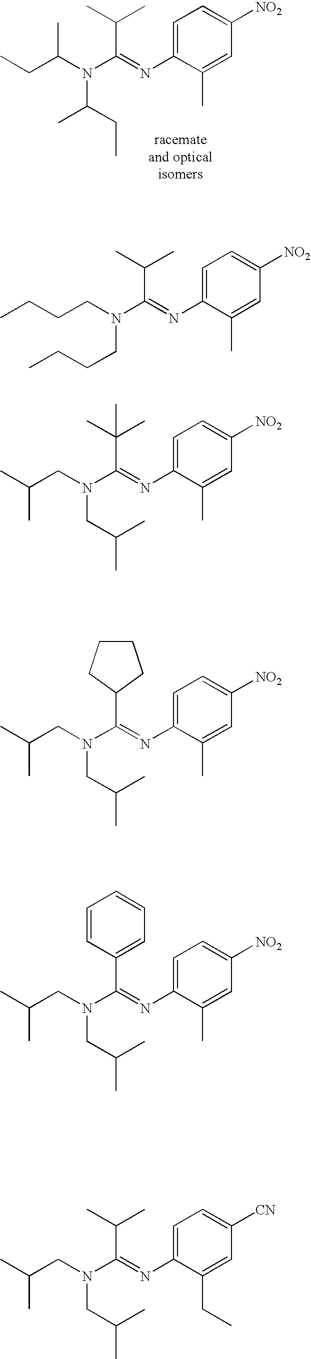 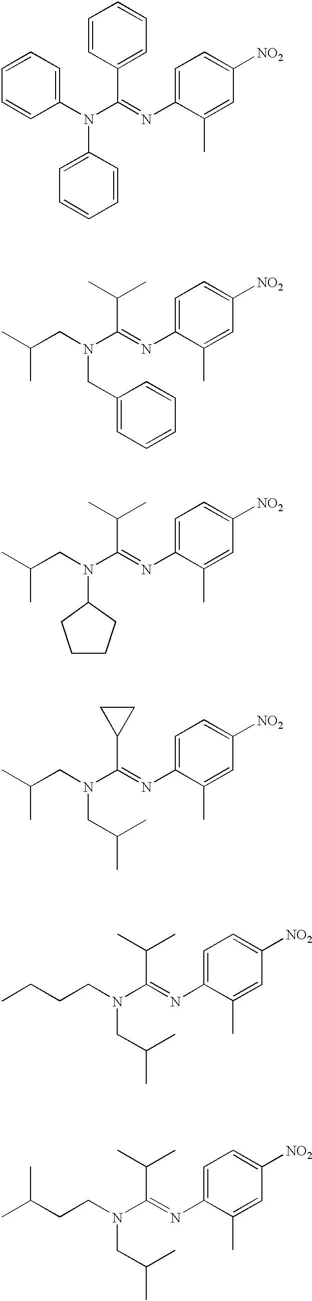 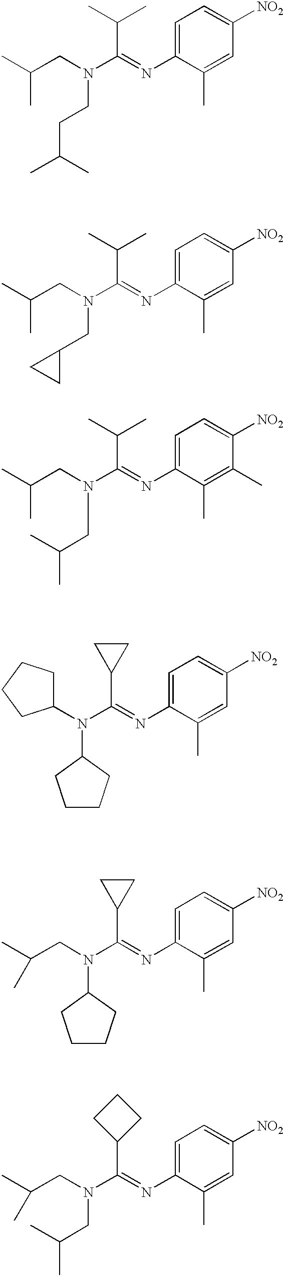
and pharmaceutically acceptable salts thereof.

20. The compound of claim 15, wherein
R¹³
is selected from the group consisting of aryl of 6-12 carbon atoms and 4-pyridyl;
R¹⁴
is selected from the group consisting of aryl of 6-12 carbon atoms, heteroaryl of 2-11 carbon atoms and 1-3 heteroatoms selected from the group consisting of N, S and O alkyl of 1-10 carbon atoms, cycloalkyl of 3-12 carbon atoms, heterocycloalkyl of 4-7 carbon atoms and 1-3 heteroatoms selected from the group consisting of N, S and O alkenyl of 2-10 carbon atoms, cycloalkenyl of 5-12 carbon atoms, with the proviso that said cycloalkenyl is mono-cyclic, and R¹⁷—R¹⁸;
T′
is selected from the group consisting of hydrogen, nitro, nitrile, alkyl of 1-6 carbon atoms, halogen, haloalkyl of 1-6 carbon atoms and a number of halogen atoms up to the perhalo level, aryl of 6-12 carbon atoms, and heteroaryl of 2-11 carbon atoms and 1-3 heteroatoms selected from the group consisting of N, S and O with the proviso that when R13 is aryl of 6-12 carbon atoms, at least one of T′ is nitro, nitrile, trifluoromethyl or halogen, or
T′
may form, together with a carbon atom adjacent to a carbon atom to which it is attached, a fused ring of 6-9 carbon atoms and 4-14 hydrogen atoms;
t′
is 1-5;
R¹⁷
is selected from the group consisting of alkyl of 1-10 carbon atoms and alkenyl of 2-10 carbon atoms;
R¹⁸
is selected from the group consisting of aryl of 6-12 carbon atoms, heteroaryl of 6-11 carbon atoms and 1-3 heteroatoms selected from the group consisting of N, S and O cycloalkyl of 3-6 carbon atoms, heterocycloalkyl of 3-6 carbon atoms and 1-3 heteroatoms selected from the group consisting of N, S and O cycloalkenyl of 3-6 carbon atoms, and heterocycloalkenyl of 3-6 carbon atoms and 1-3 heteroatoms selected from the group consisting of N, S and O;
R¹⁵ and R¹⁶
are joined to form, together with the nitrogen atom to which they are attached, a 5-8 membered ring containing 4-7 carbon atoms and 1-2 heteroatoms selected from the group consisting of N, S and O which ng may be substituted with R²¹ and R²², with the proviso that when R¹⁵ and R¹⁶ form a morpholine ring together with the nitrogen atom to which they are attached, said morpholine ring is substituted with at least one of R²¹ and R²²;
R²¹ and R²²
(i) are each independently selected from the group consisting of hydrogen, haloalkyl of 1-10 carbon atoms and a number of halogen atoms up to the perhalo level, alkyl of 1-10 carbon atoms, aryl of 6-12 carbon atoms, heteroaryl of 2-11 carbon atoms and 1-3 heteroatoms selected from the group consisting of N, S and O and benzimidazolinone, or
(ii) each independently forms, together with the carbon atom adjacent to the carbon atom to which it is attached, a fused ring of 3-6 carbon atoms and 4-10 hydrogen atoms, or
(iii) R²¹ is selected from the group consisting hydrogen, haloalkyl of 1-10 carbon atoms and a number of halogen atoms up to the perhalo level, alkyl of 1-10 carbon atoms, aryl of 6-12 carbon atoms, heteroaryl of 2-11 carbon atoms and 1-3 heteroatoms selected from the group consisting of N, S and O and benzimidazolinone and R²² forms, together with the carbon atom adjacent to the carbon atom to which it is attached, a fused ring of 3-6 carbon atoms and 4-10 hydrogen atoms;
and pharmaceutically acceptable salts thereof.

21. The compound according to claim 20, wherein
R¹³
is selected from the group consisting of aryl of 6-12 carbon atoms and 4-pyridyl;
R¹⁴
is selected from the group consisting of aryl of 6-12 carbon atoms, heteroaryl of 2-11 carbon atoms and 1-3 heteroatoms selected from the group consisting of N, S and O alkyl of 1-10 carbon atoms, cycloalkyl of 3-12 carbon atoms, heterocycloalkyl of 4-7 carbon atoms and 1-3 heteroatoms selected from the group consisting of N, S and O alkenyl of 2-10 carbon atoms, cycloalkenyl of 5-12 carbon atoms, with the proviso that said cycloalkenyl is mono-cyclic, and R¹⁷—R¹⁸;
T′
is selected from the group consisting of nitro, nitrile, trifluoromethyl and halogen;
t′
is 1-3;
R¹⁷
is selected from the group consisting of alkyl of 1-10 carbon atoms and alkenyl of 2-10 carbon atoms;
R¹⁸
is selected from the group consisting of aryl of 6-12 carbon atoms, heteroaryl of 2-11 carbon atoms and 1-3 heteroatoms selected from the group consisting of N, S and O cycloalkyl of 3-12 carbon atoms, heterocycloalkyl of 4-7 carbon atoms and 1-3 heteroatoms selected from the group consisting of N, S and O, and cycloalkenyl of 5-12 carbon atoms;
R¹⁵ and R¹⁶
are joined to form, together with the nitrogen atom to which they are attached, a 5-8 membered ring containing 4-7 carbon atoms and 1-2 heteroatoms selected from the group consisting of N, S and O which ring may optionally be substituted with R²¹ and R²², with the proviso that when R¹⁵ and R¹⁶ form a morpholine ring together with the nitrogen atom to which they are attached, said morpholine ring is substituted with at least one of R²¹ and R²²;
R²¹ and R²²
(i) are each independently selected from the group consisting of hydrogen, haloalkyl of 1-10 carbon atoms and a number of halogen atoms up to the perhalo level, alkyl of 1-10 carbon atoms, aryl of 6-12 carbon atoms, heteroaryl of 2-11 carbon atoms and 1-3 heteroatoms selected from the group consisting of N, S and O and benzimidazolinone, or
(ii) each independently forms, together with the carbon atom adjacent to the carbon atom to which it is attached, a fused ling of 3-6 carbon atoms and 4-10 hydrogen atoms, or
(iii) R²¹ is selected from the group consisting hydrogen, haloalkyl of 1-10 carbon atoms and a number of halogen atoms up to the perhalo level, alkyl of 1-10 carbon atoms, aryl of 6-12 carbon atoms, heteroaryl of 2-11 carbon atoms and 1-3 heteroatoms selected from the group consisting of N, S and O and benzimidazolinone and R²² forms, together with the carbon atom adjacent to the carbon atom to which it is attached, a fuised ring of 3-6 carbon atoms and 4-10 hydrogen atoms;
and pharmaceutically acceptable salts thereof.

22. The compound of claim 21, wherein
R¹³
is aryl of 6-12 carbon atoms;
R¹⁴
is selected from the group consisting of aryl of 6-12 carbon atoms, heteroaryl of 2-11 carbon atoms and 1-3 heteroatoms selected from the group consisting of N, S and O alkyl of 1-10 carbon atoms, cycloalkyl of 3-12 carbon atoms, heterocycloalkyl of 4-7 carbon atoms and 1-3 heteroatoms selected from the group consisting of N, S and O alkenyl of 2-10 carbon atoms, cycloalkenyl of 5-12 carbon atoms, with the proviso that said cycloalkenyl is mono-cyclic, and R¹⁷—R¹⁸
T′
is selected from the group consisting of nitro, nitrile, trifluoromethyl and halogen;
t′
is 1-3;
R¹⁵ and R¹⁶
are joined to form, together with the nitrogen atom to which they are attached, a 5-8 membered ring containing 4-7 carbon atoms and 1-2 heteroatoms selected from the group consisting of N, S and O which ring maybe substituted with R²¹ and R²², with the proviso that when R¹⁵ and R¹⁶ form a morpholine ring together with the nitrogen atom to which they are attached, said morpholine rng is substituted with at least one of R²¹ and R²²;
R²² and R²²
(i) are each independently selected from the group consisting of hydrogen, haloalkyl of 1-10 carbon atoms and a number of halogen atoms up to the perhalo level, alkyl of 1-10 carbon atoms, aryl of 6-12 carbon atoms, heteroaryl of 2-11 carbon atoms and 1-3 heteroatoms selected from the group consisting of N, S and O and benzimidazolinone, or
(ii) each independently forms, together with the carbon atom adjacent to the carbon atom to which it is attached, a fused ring of 3-6 carbon atoms and 4-10 hydrogen atoms, or
(iii) R²¹ is selected from the group consisting hydrogen, haloalkyl of 1-10 carbon atoms and a number of halogen atoms up to the perhalo level, alkyl of 1-10 carbon atoms, aryl of 6-12 carbon atoms, heteroaryl of 2-11 carbon atoms and 1-3 heteroatoms selected from the group consisting of N, S and O and benzimidazolinone and R²² forms, together with the carbon atom adjacent to the carbon atom to which it is attached, a fiised ring of 3-6 carbon atoms and 4-10 hydrogen atoms;
and pharmaceutically acceptable salts thereof

23. The compound of claim 20, selected from the group consisting of 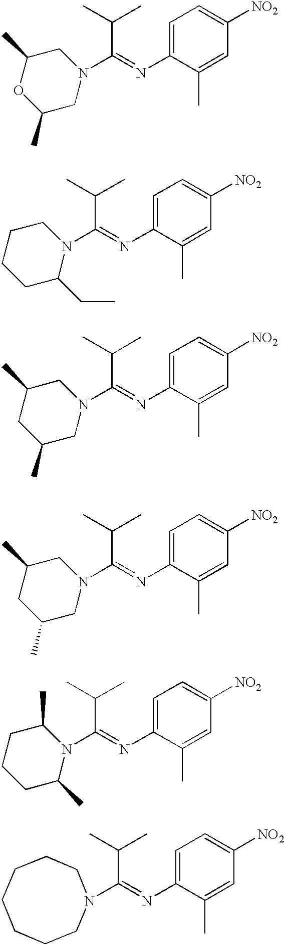
and pharmaceutically acceptable salts thereof.

24. A pharmaceutical composition comprising a compound of claim 1 or 15 and a pharmaceutically acceptable carrier.

25. A method of treating a disease or condition in a mammal, comprising administering to a mammal in need thereof an effective amount of a compound according to the general formula (III) 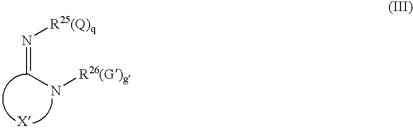
wherein
R²⁵
is selected from the group consisting of aryl of 6-12 carbon atoms and heteroaryl of 2-11 carbon atoms and 1-3 heteroatoms selected from the group consisting of N, S and O;
Q
is selected from the group consisting of hydrogen, nitro, nitrile, alkyl of 1-6 carbon atoms, halogen, haloalkyl of 1-6 carbon atoms and a number of halogen atoms up to the perhalo level, aryl of 6-12 carbon atoms, and heteroaryl of 2-11 carbon atoms and 1-3 heteroatoms selected from the group consisting of N, S and O or
Q
may fonn, together with a carbon atom adjacent to a carbon atom to which it is attached, a fused ring of 6-9 carbon atoms and 4-14 hydrogen atoms;
q
is 0-5;
R²⁶
is selected from the group consisting of hydrogen, alkyl of 1-10 carbon atoms, haloalkyl of 1-10 carbon atoms and a number of halogen atoms up to the perhalo level, cycloalkyl of 3-12 carbon atoms, heterocycloalkyl of 4-7 carbon atoms and 1-3 heteroatoms selected from the group consisting of N, S and O alkenyl of 2-10 carbon atoms, cycloalkenyl of 5-12 carbon atoms and containing 1-3 rings, and alkynyl of 3-10 carbon atoms;
G′
is a selected from the group consisting of hydrogen, nitro, nitrile, halogen, OH, OR²⁷, ═O, haloalkyl of 1-4 carbon atoms and a number of halogen atoms up to the perhalo level, alkyl of 1-4 carbon atoms, alkenyl of 1-4 carbon atoms, cycloalkyl of 3-7 carbon atoms, heterocycloalkyl of 3-5 carbon atoms and 1-3 heteroatoms selected from the group consisting of N, O, and S, cycloalkenyl of 5-7 carbon atoms, heterocycloalkenyl of 4-6 carbon atoms and 1-3 heteroatoms selected from the group consisting of N, O, and S, CO₂R²⁷, C(O)N(R²⁸)(R²⁹), aryl of 6-10 carbon atoms, heteroaryl of 3-9 carbon atoms and 1-3 heteroatoms selected from the group consisting of N, O, and S, S(O)_{y}R³⁰, SO₃R³⁰, and SO₂N(R²⁸)(R²⁹);
R²⁷
is selected from the group consisting of alkyl of 1-4 carbon atoms, haloalkyl of 1-4 carbon atoms and a number of halogen atoms up to the perhalo level, cycloalkyl of 3-6 carbon atoms, and halocycloalkyl of 3-6 carbon atoms;
R²⁸ and R²⁹
are each independently selected from the group consisting of hydrogen and alkyl of 1-5 carbon atoms;
R³⁰
is selected from the group consisting of alyl of 1-5 carbon atoms, SO₂F, CHO, OH, nitro, nitrile, halogen, OCF₃, N-oxide, O—C(R³¹)₂O, C(O)NHC(O), the carbon atoms being connected to adjacent positions on R, and C(O)C₆H₄, the carbonyl carbon and the ring carbon ortho to the carbonyl carbon being connected to adjacent positions on R;
R³¹
is selected from the group consisting of hydrogen, halogen and alkyl of 1-4 carbon atoms;
y
is 0-2
g′
is 0-4, with the exception of halogen, which may be employed up to the perhalo level;
provided that when G is alkyl of 1-4 carbon atoms, alkenyl of 1-4 carbon atoms, cycloalkyl of 3-7 carbon atoms, heterocycloalkyl of 3-5 carbon atoms, cycloalkenyl of 5-7 carbon atoms, or heterocycloalkenyl of 4-6 carbon atoms, then G optionally may bear secondary substituents of halogen up to the perhalo level; and when G is aryl or heteroaryl, then G optionally may bear secondary substituents independently selected from the group consisting of alkyl of 1-4 carbon atoms and halogen, the number of said secondary substituents being up to 3 for alkyl moieties, and up to the perhalo level for halogen;
X′forms, together with the nitrogen atom and carbon atom to which it is attached, a polycyclic ring structure of containing 3-4 rings, wherein each ring contains 3-8 carbon atoms and may optionally be substituted with one or more of alkyl of 1-6 carbon atoms or alkenyl of 2-6 carbon atoms, or
X′ is 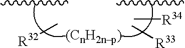
wherein binding is achieved via the terminal carbon atoms;
n
is 3-7;
p
is 0-7;
R³², R³³ and R³⁴
(i) are each independently selected from the group consisting of hydrogen, haloalkyl of 1-10 carbon atoms and a number of halogen atoms up to the perhalo level, alkyl of 1-10 carbon atoms, cycloalkyl of 3-12 carbon atoms, alkenyl of 2-10 carbon atoms, cycloalkenyl of 5-7 carbon atoms, and alkynyl of 3-10 carbon atoms, or
(ii) R³² and R³³ are each independently selected from the group consisting of hydrogen, haloalkyl of 1-10 carbon atoms and a number of halogen atoms up to the perhalo level, alkyl of 1-10 carbon atoms, cycloalkyl of 3-12 carbon atoms, alkenyl of 2-10 carbon atoms, cycloalkenyl of 5-7 carbon atoms, and alkynyl of 3-10 carbon atoms and R³⁴ forms, together with the carbon atom to which it is attached, a spiro ring of 3-6 carbon atoms, or
(iii) R³² and R³³ are each independently selected from the group consisting of hydrogen, haloalkyl of 1-10 carbon atoms and a number of halogen atoms up to the perhalo level, alkyl of 1-10 carbon atoms, cycloalkyl of 3-12 carbon atoms, alkenyl of 2-10 carbon atoms, cycloalenyl of 5-7 carbon atoms, and alkynyl of 3-10 carbon atoms and R³⁴ forms, together with the carbon atom adjacent to the carbon atom to which it is attached, a fused ring of 3-7 carbon atoms and 4-14 hydrogen atoms, or
(iv) R³² and R³³ are each independently selected from the group consisting of hydrogen, haloalkyl of 1-10 carbon atoms and a number of halogen atoms up to the perhalo level, alkyl of 1-10 carbon atoms, cycloalkyl of 3-12 carbon atoms, alkenyl of 2-10 carbon atoms, cycloalkenyl of 5-7 carbon atoms, and alkynyl of 3-10 carbon atoms and R³⁴ forms, together with the carbon atom that is 2-4 carbon atoms away from the carbon atom to which it is attached, a fused ring of 3-7 carbon atoms and 4-14 hydrogen atoms; and pharmaceutically acceptable salts thereof for:
A1) enhancement of bone formation in bone weakening diseases for the treatment or prevention of osteopenia or osteoporosis;
A2) enhancement of fracture healing;
B1) use as a female contragestive agent;
B2) prevention of endometrial implantation;
B3) induction of labor;
B4) treatment of luteal deficiency;
B5) enhanced recognition and maintanence of pregnancy;
B6) counteracting of preeclampsia, eclampsia of pregnancy, and preterm labor;
B7) treatment of infertility, including promotion of spermatogenesis, induction of the acrosome reaction, maturation of oocytes, or in vitro fertilization of oocytes;
C1) treatment of dysmenorrhea;
C2) treatment of dysfunctional uterine bleeding;
C3) treatment of ovarian hyperandrogynism;
C4) treatment of ovarian hyperaldosteronism;
C5) alleviation of premenstral syndrome and of premenstral tension;
C6) alleviation of perimenstrual behavior disorders;
C7) treatment of climeracteric disturbance, including, menopause transition, mood changes, sleep disturbance, and vaginal dryness;
C8) enhancement of female sexual receptivity and male sexual receptivity;
C9) treatment of post menopausal urinary incontinence;
C10) improvement of sensory and motor functions;
C11) improvement of short term memory;
C12) alleviation of postpartum depression;
C13) treatment of genital atrophy;
C14) prevention of postsurgical adhesion formation;
C15) regulation of uterine immune function;
C16) prevention of myocardial infarction;
D1) hormone replacement;
E1) treatment of cancers, including hormone mediated cancers, such as breast cancer, uterine cancer, ovarian cancer, and endometrial cancer;
E2) treatment of endometriosis;
E3) treatment of uterine fibroids;
F1) treatment of hirsutism;
F2) inhibition of hair growth;
G1) activity as a male contraceptive;
G2) activity as an abortifacient; and
H1) promotion of mylin repair.

26. The method of claim 25, wherein said disease or condition is selected from osteopenia and osteoporosis.

27. The method of claim 25, wherein said disease or condition is bone fracture.

28. The method of claim 25, wherein said compound is administered for use as a female contragestive agent.

29. The method of claim 25, wherein said compound is administered for use as a hormone replacement.

30. A method of treating a disease or condition in a mammal, comprising administering to a mammal in need thereof an effective amount of a compound according to the general formula (IV) 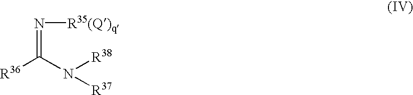
wherein
R³⁵
is selected from the group consisting of aryl of 6-12 carbon atoms and heteroaryl of 2-11 carbon atoms and 1-3 heteroatoms selected from the group consisting of N, S and O;
R³⁶
is selected from the group consisting of aryl of 6-12 carbon atoms, heteroaryl of 2-11 carbon atoms and 1-3 heteroatoms selected from the group consisting of N, S and O alkyl of 1-10 carbon atoms, cycloalkyl of 3-12 carbon atoms, heterocycloalkyl of 4-7 carbon atoms and 1-3 heteroatoms selected from the group consisting of N, S and O alkenyl of 2-10 carbon atoms, cycloalkenyl of 5-12 carbon atoms, and R³⁹—R⁴⁰;
Q′
is selected from the group consisting of hydrogen, nitro, nitrile, alkyl of 1-6 carbon atoms, halogen, haloalkyl of 1-6 carbon atoms and a number of halogen atoms up to the perhalo level, aryl of 6-12 carbon atoms, and heteroaryl of 2-11 carbon atoms and 1-3 heteroatoms selected from the group consisting of N, S and O or
Q′
may form, together with a carbon atom adjacent to a carbon atom to which it is attached, a fused ring of 6-9 carbon atoms and 4-14 hydrogen atoms;
q′
is 0-5;
R³⁹
is selected from the group consisting of alkyl of 1-10 carbon atoms and alkenyl of 2-10 carbon atoms;
R⁴⁰
is selected from the group consisting of aryl of 6-12 carbon atoms, heteroaryl of 2-11 carbon atoms and 1-3 heteroatoms selected from the group consisting of N, S and O cycloalkyl of 3-12 carbon atoms, heterocycloalkyl of 4-7 carbon atoms and 1-3 heteroatoms selected from the group consisting of N, S and O, and cycloalkenyl of 5-12 carbon atoms;
R³⁷ and R³⁸
(i) are each independently selected from the group consisting of hydrogen, aryl of 6-12 carbon atoms, heteroaryl of 2-11 carbon atoms and 1-3 heteroatoms selected from the group consisting of N, S and O alkyl of 1-10 carbon atoms, cycloalkyl of 3-12 carbon atoms, heterocycloalkyl of 4-7 carbon atoms and 1-3 heteroatoms selected from the group consisting of N, S and O alkenyl of 2-10 carbon atoms, cycloalkenyl of 5-12 carbon atoms, and R⁴¹—R⁴², such that the total number of atoms in R³⁶, R³⁷ and R³⁸ is greater than or equal to 9, or
(ii) are joined to form, together with the nitrogen atom to which they are attached, a 5-8 membered ring containing 4-7 carbon atoms and 1-2 heteroatoms selected from the group consisting of N, S and O which ring may optionally be substituted with R⁴³ and R⁴⁴;
R⁴¹
is selected from the group consisting of alkyl of 1-10 carbon atoms, cycloalkyl of 3-12 carbon atoms, heterocycloalkyl of 4-7 carbon atoms and 1-3 heteroatoms selected from the group consisting of N, S and O alkenyl of 2-10 carbon atoms, cycloalkenyl of 5-12 carbon atoms, and alkynyl of 3-10 carbon atoms;
R⁴²
is selected from the group consisting of hydrogen, aryl of 6-12 carbon atoms, heteroaryl of 2-11 carbon atoms and 1-3 heteroatoms selected from the group consisting of N, S and O cycloalkyl of 3-12 carbon atoms, heterocycloalkyl of 4-7 carbon atoms and 1-3 heteroatoms selected from the group consisting of N, S and O cycloalkenyl of 5-12 carbon atoms, and R⁴⁵—R⁴⁶;
R⁴⁵
is selected from the group consisting of aryl of 6-12 carbon atoms and heteroaryl of 2-11 carbon atoms and 1-3 heteroatoms selected from the group consisting of N, S and O;
R⁴⁶
is selected from the group consisting of hydrogen, halogen, nitrile, nitro, alkyl of 1-10 carbon atoms, and haloalkyl of 1-6 carbon atoms and 1-3 halo atoms;
R⁴³ and R⁴⁴
(i) are each independently selected from the group consisting of hydrogen, haloalkyl of 1-10 carbon atoms and a number of halogen atoms up to the perhalo level, alkyl of 1-10 carbon atoms, aryl of 6-12 carbon atoms, heteroaryl of 2-11 carbon atoms and 1-3 heteroatoms selected from the group consisting of N, S and O and benzimidazolinone, or
(ii) each independently forms, together with the carbon atom adjacent to the carbon atom to which it is attached, a fused ring of 3-6 carbon atoms and 4-10 hydrogen atoms, or
(iii) R⁴³ is selected from the group consisting hydrogen, haloalkyl of 1-10 carbon atoms and a number of halogen atoms up to the perhalo level, alkyl of 1-10 carbon atoms, aryl of 6-12 carbon atoms, heteroaryl of 2-11 carbon atoms and 1-3 heteroatoms selected from the group consisting of N, S and O and benzimidazolinone and Re forms, together with the carbon atom adjacent to the carbon atom to which it is attached, a fused ring of 3-6 carbon atoms and 4-10 hydrogen atoms;
and pharmaceutically acceptable salts thereof for:
A1) enhancement of bone formation in bone weakening diseases for the treatment or prevention of osteopenia or osteoporosis;
A2) enhancement of fracture healing;
B1) use as a female contragestive agent;
B2) prevention of endometrial implantation;
B3) induction of labor;
B4) treatment of luteal deficiency;
B5) enhanced recognition and maintanence of pregnancy;
B6) counteracting of preeclampsia, eclampsia of pregnancy, and preterm labor;
B7) treatment of infertility, including promotion of spermatogenesis, induction of the acrosome reaction, maturation of oocytes, or in vitro fertilization of oocytes;
C1) treatment of dysmenorrhea;
C2) treatment of dysfunctional uterine bleeding;
C3) treatment of ovarian hyperandrogynism;
C4) treatment of ovarian hyperaldosteronism;
C5) alleviation of premenstral syndrome and of premenstral tension;
C6) alleviation of perimenstrual behavior disorders;
C7) treatment of climeracteric disturbance, including. menopause transition, mood changes, sleep disturbance, and vaginal dryness;
C8) enhancement of female sexual receptivity and male sexual receptivity;
C9) treatment of post menopausal urinary incontinence;
C10) improvement of sensory and motor functions;
C b 11) improvement of short term memory;
C12) alleviation of postpartum depression;
C13) treatment of genital atrophy;
C14) prevention of postsurgical adhesion formation;
C15) regulation of uterine immune fimction;
C16) prevention of myocardial infarction;
D1) hormone replacement;
E1) treatment of cancers, including hormone mediated cancers, such as breast cancer, uterine cancer, ovarian cancer, and endometrial cancer;
E2) treatment of endometriosis;
E3) treatment of uterine fibroids;
F1) treatment of hirsutism;
F2) inhibition of hair growth;
G1) activity as a male contraceptive;
G2) activity as an abortifacient; and
H1) promotion of mylin repair.

31. The method of claim 30, wherein said disease or condition is selected from osteopenia and osteoporosis.

32. The method of claim 30, wherein said disease or condition is bone fracture.

33. The method of claim 30, wherein said compound is administered for use as a female contragestive agent.

34. The method of claim 30, wherein said compound is administered for use as a hormone replacement.
